(19) United States
(12) Patent Application Publication
(10) Pub. No.: US 2010/0081621 A1
(43) Pub. Date: Apr. 1, 2010

(54) Crystal structure of the catalytic domain of the viral restriction factor APOBEC3G

(76) Inventors: Lauren Holden, Los Angeles, CA (US); Courtney Prochnow, Los Angeles, CA (US); Y. Paul Chang, Los Angeles, CA (US); Ronda Bransteitter, Culver City, CA (US); Linda Chelico, Los Angeles, CA (US); Udayaditya Sen, Los Angeles, CA (US); Myron F. Goodman, Los Angeles, CA (US); Xiaojiang S. Chen, Los Angeles, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(21) Appl. No.: 12/583,141

(22) Filed: Aug. 14, 2009

Related U.S. Application Data
(60) Provisional application No. 61/089,141, filed on Aug. 15, 2008.

(51) Int. Cl. A61K 38/04; G01N 33/566; G06G 7/48; G01N 33/53; C12Q 1/68; A61K 31/7088

(52) U.S. Cl. 514/12; 436/501; 703/11; 435/7.1; 435/6; 514/44 R

(57) ABSTRACT

The structure, function and methods associated with proteins from the APOBEC family, which are involved in diverse biological functions, is disclosed. In one embodiment, the structure of APOBEC-3G (Apo3G) is disclosed. In another embodiment, a method of using APOBEC-3G (Apo3G) and/or Apo3G-CD2 to restrict the replication of Human Immunodeficiency Virus (HIV) and Hepatitis B virus (HBV) via cytidine deamination on ssDNA or RNA binding is disclosed. In yet another embodiment, the high-resolution crystal structure of an enzymatically active APOBEC protein, the C-terminal deaminase domain of Apo3G (Apo3G-CD2) is disclosed.

### CLAIM OF PRIORITY

This application claims priority to U.S. Provisional Application No. 61/089,141, filed Aug. 15, 2008, the entire contents of which are incorporated herein.

### RELATED APPLICATION

This application is related to U.S. Application No. 61/016,172, filed on Dec. 21, 2007.

### GOVERNMENT SUPPORT

This invention was made with government support under Contract No. R01 AI050096 awarded by the National Institutes of Health. The government has certain rights in this invention.

### BACKGROUND

1. Field of Disclosure

The present disclosure relates generally to the information provided by the three-dimensional structure of the C-terminal domain of APOBEC3G (Apo3G-CD2) and other structure models of any APOBEC proteins obtained by computer modeling that bears similarity with a root-mean-square deviation (RMSD) of 2.0 with the Apo3G-CD2 monomer. Additionally, the present disclosure relates to the uses of the three-dimensional structure of Apo3G-CD2 and models of APOBEC proteins particularly for structure-based drug design of compounds, peptides or mutant APOBEC proteins designed to treat Hyper-IgM-2 Syndrome, B cell lymphomas and lentivirus infections, particularly the human immunodeficiency virus (HIV) infection.

2. General Background

The present disclosure relates to the APOBEC family members, which are involved in diverse biological functions. APOBEC-3G (Apo3G) restricts the replication of Human Immunodeficiency Virus (HIV) and Hepatitis B virus (HBV) via cytidine deamination on ssDNA or RNA binding. The present disclosure also related to the high-resolution crystal structure of an enzymatically active APOBEC protein, the C-terminal deaminase domain of Apo3G (Apo3G-CD2). The Apo3G-CD2 structure closely resembles the Apo2 structure and a detailed comparison suggests that differences in the loops near the active center influence substrate binding and activity. The Apo3G-CD2 structure differs significantly from a recently reported NMR structure of the A3G-CD2 mutant. The NMR structure lacks features, including the absence of a helical region (helix 1) and an intact β strand (β2), which may significantly contribute to the active center conformation and oligomer formation. The loops in the X-ray structure of Apo3G-CD2 are in open conformations around the active site and form a continuous “substrate groove” that can accommodate a ssDNA substrate. We have introduced mutations around the groove that identify critical residues involved in substrate specificity, ssDNA binding, and deaminase activity. The structure permits the modeling of the full-length Apo3G and provides insights into key residues and structural features that are important for HIV viral incorporation and viral restriction.

### SUMMARY

The apolipoprotein B mRNA-editing enzyme catalytic polypeptide (APOBEC)-3G (Apo3G, previously named CEM15) was discovered in a subtractive hybridization screen as the cellular factor that blocks the replication of a human immunodeficiency virus type-1 (HIV-1) strain that is deficient for its viral infectivity factor (Vif) protein (Chiu and Greene, 2007; Conticello et al., 2007b; Holmes et al., 2007). The HIV-1 expresses its Vif protein to overcome the Apo3G imposed replication block primarily by binding to Apo3G and targeting it for polyubiquitylation and proteasomal degradation (Chiu and Greene, 2007; Conticello et al., 2007b; Holmes et al., 2007). In the absence of Vif, Apo3G multimers associated with viral RNA are packaged into budding HIV-1 virions (Burnett and Spearman, 2007). When these virions enter new target cells, Apo3G introduces multiple cytidine deaminations on the HIV-1 minus strand cDNA to inactivate the provirus and block infection (Suspene et al., 2004; Yu et al., 2004). Apo3G can also disrupt the HIV-1 reverse transcription (RT) process (Guo et al., 2007; Iwatani et al., 2007; Xiao-Yu et al., 2007) and impair the integration of the provirus (Luo et al., 2007; Mbisa et al., 2007). Beyond HIV-1, Apo3G can inhibit other retroviruses, retrotransposons and the Hepatitis B Virus (HBV) (Chiu and Greene, 2007; Conticello et al., 2007b; Holmes et al., 2007). Although non-catalytic properties of Apo3G are significant (Chiu and Greene, 2007), recent reports show that the catalytic activity of Apo3G is necessary for efficient restriction of HIV-1 and retrotransposition when Apo3G is expressed at endogenous levels (Miyagi et al., 2007; Schumacher et al., 2008).

Apo3G belongs to the APOBEC family of polynucleotide cytidine deaminase enzymes including: APOBEC-1 (Apo1), APOBEC-2 (Apo2), APOBEC-3A-APOBEC-3H (Apo3A-Apo3H), APOBEC-4 (Apo4) and activation induced cytidine deaminase (AID). These enzymes have one or two conserved cytidine deaminase motifs defined as H-X-E-X₂₃₂₈-P-C-X₂₄-C (X=any amino acid) and achieve remarkably diverse functions by binding or deaminating single-stranded (ss) DNA and RNA (Chiu and Greene, 2007; Conticello et al., 2007b; Holmes et al., 2007). The first discovered APOBEC protein, Apo-1, deaminates the 6666 cytidine in the apolipoprotein B mRNA thereby creating a premature stop codon leading to the formation of two protein isoforms with distinct roles in lipid metabolism (Conticello et al., 2007b). Cytidine deamination catalyzed by AID on the immunoglobulin gene during somatic hypermutation and class switch recombination is required for antibody affinity maturation (Bransteitter et al., 2006; Conticello et al., 2007b; Peled et al., 2007). The APOBEC-3 proteins inhibit retroviruses, various retrotransposons and some DNA viruses, such as the hepatitis B virus (HBV) and the adeno-associated virus (AAV) (Chiu and Greene, 2007; Conticello et al., 2007b; Holmes et al., 2007).

Attempts to understand the biochemical mechanisms of the APOBEC proteins from a structural perspective have involved comparative modeling with other related zinc coordinating deaminases that deaminate free cytidine nucleotide bases (Jarmuz et al., 2002; Navaratnam et al., 1998; Wedekind et al., 2003; Xie et al., 2004). Originally, a homology model of Apo-1 was created based on the square-shaped dimer structure of the Escherichia coli cytidine deaminase (ECDA) (Betts et al., 1994; Navaratnam et al., 1998). The active centers of an ECDA dimer, which consist of residues from different monomers, are buried and accessible only to small free nucleotide substrates. Apo1 was modeled to have the same structural organization as ECDA, with one catalytic active site region, a linker region and a pseudoactive site region. Sequence alignments of the newly discovered APOBEC proteins with Apo1 led to the same domain organization classification and oligomerization mode (Jarmuz et al., 2002; Navaratnam et al., 1998; Wedekind et al., 2003). Later, similar homology modeling of AID and Apo3G were attempted based on the Saccharomyces cerevisiae CDD1 cytidine deaminase (ScCDD1) structure that forms a square-shaped tetramer (Wedekind et al., 2003; Xie et al., 2004). Yet, similar to the ECDA, the active sites of the ScCDD1 square-like tetramer are buried and only accessible to free nucleotides, which is the known substrate En vivo. However, ScCDD1 is reported to deaminate the apoB mRNA in a yeast cell based assay (Dance et al., 2001). Upon removal of two neighboring molecules within the ScCDD1 tetramer structure, the active sites of the resulting ScCDD1 dimer are more accessible to larger nucleic acid substrates, which may provide an explanation as to how ScCDD1 can deaminate the apoB mRNA substrate in vitro.

Previously, we solved the first high-resolution crystal structure of an APOBEC protein, Apo2 (Prochnow et al., 2007). Many of the structural features of Apo2 are highly conserved among all of the Zn-deaminase superfamily members. However, in striking contrast to the square-shaped oligomers of the ECDA and ScCDD1, Apo2 forms a rod-shaped tetramer. Unique structural features of Apo2 prevent the square-shaped oligomerization and facilitate the formation of the elongated oligomer (Prochnow et al., 2007). Small-x ray scattering (SAXS) data of Apo3G dimers provides supporting evidence that other APOBECs have a similar elongated oligomerization (Chelico and Goodman, 2008; Wedekind et al., 2006). Although deamination activity of Apo2 has not yet been observed, the structure shows how the APOBEC active sites are accessible to DNA or RNA. To better understand how the APOBEC proteins act on their substrates, it is important to obtain additional structures of APOBEC proteins that are enzymatically characterized. Here, we report the high resolution crystal structure of a truncated Apo3G protein that consists of the enzymatically active CD2 domain. The surface representation of the Apo3G structure reveals a substrate binding “groove”. With structure-based mutagenesis, we identify residues within and near the groove that are important for substrate interactions and deaminase activity. The combination of structural and biochemical results provide a foundation for understanding how APOBEC family proteins bind nucleic acids, recognize substrates, and form oligomers.

APOBEC-2 (Apo2) belongs to the Apolioprotein B (APOB) mRNA-editing enzyme catalytic polypeptide (APOBEC) family of cytidine deaminases found exclusively in vertebrates (6). APOBEC nucleic acid deaminases modify genes by deaminating cytosines in mRNA coding sequences and in single-stranded DNA (6). Additionally, these enzymes can inhibit the replication of retroviruses, such as the human immunodeficiency virus (HIV) and hepatitis B virus (HBV), and retrotransposons. (4,5,6,7).

The APOBEC family is composed of APOBEC-1 (Apo1), APOBEC-2, Activation Induced Cytidine Deaminase (AID), APOBEC-3 (3A, 3B, 3C, 3DE, 3F, 3G, and 3H) and APOBEC-4 (2). Apo1, the first member to be characterized, deaminates C⁶⁶⁶⁶→U in the APOB mRNA thereby creating a premature stop codon, which results in a truncated APOB100 protein (APOB48) with a different function. Of the APOBEC3 subgroup of enzymes, APOBEC-3B (A3B), APOBEC-3F (A3F) and APOBEC-3G (A3G) have two cytidine deaminase domains (CDAs) and inhibit HIV-1 replication in the absence of the HIV viral infectivity factor protein (Vif) (4,5,6,7). In this setting, the APOBEC enzymes are incorporated into HIV virions and introduce multiple dC→dU deaminations on the minus strand of HIV viral cDNA formed during reverse transcription. Additionally, APOBEC enzymes inhibit HIV replication by a less characterized mechanism that is independent of deamination activity. APOBEC3 proteins also shield the human genome from the deleterious action of endogenous retrotransposons: A3A, A3B, A3C and A3F inhibit LINE 1 and Alu retrotransposition.

AID and Apo2 have a single CDA homology domain and are phylogenetically the most ancient members of the APOBEC family (2). AID induces somatic hypermutation (SHM) and class switch recombination (CSR) in activated germinal center B cells (3). Specific point mutations in AID are responsible for an immunodeficiency disease, Hyper-IgM-2 (HIGM-2) syndrome, which is characterized by a deficiency in isotype-switched and high affinity antibody formation (14,15). Additionally, aberrant expression of AID can induce B cell lymphomas (1,29).

Apo2, also known as ARCD-1, is ubiquitously expressed at low levels in both human and mouse and highly expressed in cardiac and skeletal muscle (16). Apo2 can form heterodimers with Apo1 and inhibit APOB mRNA deamination by Apo1 (16). Apo2 is encapsulated into HIV-1 virions when co-expressed with Δvif HIV-1 DNA in 293T cells (21). However, studies fail to show that Apo2 inhibits HIV-1 viral replication (21).

The APOBEC proteins use the same deamination activity and RNA binding properties to achieve diverse human biological functions. A comprehension of the molecular mechanisms of the APOBEC enzymes has been limited by the lack of 3-dimensional structures. Therefore, there is a need in the art for solving a 3-dimensional structure of Apo3G-CD2 and creating 3-dimensional models of other APOBEC enzymes derived from the Apo3G-CD2 structure.

Patients diagnosed with Hyper-IgM-2 Syndrome suffer from severe and recurrent infections throughout their lifetime. Currently, the only cure for Hyper-IgM-2 Syndrome is a bone marrow transplant if it is possible. The only treatment available is lifelong immunoglobulin replacement therapy. Given that mutations in the gene encoding the APOBEC protein, AID, cause Hyper-IgM-2 Syndrome, there is a need in the art for using information provided by the 3-dimensional structure of an APOBEC protein (such as Apo3G-CD2) to design drugs or mutant AID enzymes to serve as a cure or treatment for this chronic disease.

There is a need in the art for using the information provided by the 3-dimensional structure of an APOBEC protein (such as Apo3G-CD2) to design drugs that can affect the deamination activity of APOBEC proteins. The aberrant expression and deamination activity of AID has been shown to result in B cell lymphoma (1,29). Drugs that can restore the proper function of APOBEC deaminases and the timing of their function could prevent or treat B cell lymphomas.

HIV is a human retrovirus which leads to the depletion of CD4+ T lymphocytes resulting in the acquired immunodeficiency syndrome (AIDS). AIDS is characterized by various pathological conditions, including immune incompetence, opportunistic infections, neurological dysfunctions, and neoplastic growth. HIV-1 relies on Vif (virion infectivity factor), a protein encoded by HIV-1 and many related primate lentiviruses, to evade the potent innate antiviral function of APOBEC3G (also known as CEM15) and APOBEC3F in vivo. Most of the APOBEC-3 proteins are DNA cytidine deaminases that are incorporated into virions and produce extensive hypermutation in newly synthesized viral DNA formed during reverse transcription. These proteins can also inhibit HIV replication by a less characterized mechanism that is independent of deamination activity but that involves RNA binding.

Despite the availability of a number of drugs to combat HIV infections, there is a need in the art for additional drugs that inhibit HIV replication, and which are suitable for treating HIV and other lentiviral infections. The present invention addresses this need by providing structure based methods for identifying agents that target APOBEC enzymes and prevent Vif mediated degradation of APOBEC3G, APOBEC3F or other APOBEC enzymes that can restrict HIV replication under certain conditions.

There is a need in the art for using the information provided by the 3-dimensional structure of an APOBEC protein (such as Apo3G-CD2) to design drugs that can affect the oligomerization of the APOBEC protein. It has been demonstrated that oligomerization of APOBEC proteins occurs in vivo and in vitro. Information provided by the Apo3G-CD2 structure suggests this oligomerization is important for the biological functions of these enzymes. Drugs designed to affect oligomerization of APOBEC enzymes may enhance or restrict their biological functions, such as, deamination activity, RNA binding properties and viral restriction.

There is a need in the art for designing or identifying compounds that mimic, enhance, disrupt or compete with the interactions of APOBEC proteins with their substrates and other cellular or viral proteins, such as HIV Vif. Knowledge of the three dimensional structure of the protein enables a skilled artisan to design a compound that has a specific and appropriate conformation to achieve such an objective. Information from the three dimensional structure of the protein also enables a skilled artisan strategically select such a compound from available libraries of compounds. For example, knowledge of the three dimensional structure of Apo3G-CD2 enables one of skill in the art to design a compound that binds to Apo3G-CD2 or other APOBEC proteins that can inhibition interactions with the HIV Vif protein and restore the ability of APOBEC proteins to restrict HIV viral replication.

### SUMMARY

One embodiment of the present disclosure provides structural information derived from the Apo3G-CD2 crystal structure and models of related APOBEC proteins obtained by computer modeling that bears similarity with a root-mean-square deviation (RMSD) of 2.0 with the Apo3G-CD2 monomer. Additionally, other embodiments of the present disclosure provide methods for using this structural information to design drugs to treat chronic diseases, such as Hyper-IgM-2 Syndrome, B cell lymphomas, and infectious lentiviral infections, such as HIV. Yet other embodiments of the present disclosure drugs and related methods to affect the DNA or RNA binding properties, zinc coordination and/or oligomerization of APOBEC proteins. Additionally, yet other embodiments of the present disclosure include drugs and related methods to inhibit interactions with other cellular or viral proteins, including but not limited to, HIV Vif. The present disclosure provides these and other additional advantages described herein.

Definitions

According to the present disclosure, the C-terminus of APOBEC3G (Apo3G-CD2) can be defined as a protein that is characterized by the amino acid sequence including amino acids 197-380. Additionally, Apo3G-CD2 can be defined as a protein including amino acids 197-380 filed in the NCBI Genbank data base(NP_{—}068594; GI: 13399304). According to the present disclosure, general reference to the Apo3G-CD2 protein is a protein that, at a minimum, includes an Apo3G-CD2 monomer and may include other biologically active fragments of APOBEC proteins.

A “homologue” of an APOBEC protein, or “homologous” APOBEC protein, includes proteins which differ from a naturally occurring APOBEC protein in that at least one or a few, but not limited to one or a few, amino acids have been deleted (e.g., a truncated version of the protein, such as a peptide or fragment), inserted, inverted, substituted and/or derivatized (e.g., by glycosylation, phosphorylation, acetylation, myristoylation, prenylation, palmitation, amidation and/or addition of glycosylphosphatidyl inositol). Preferably, an APOBEC homologue has a buried amino acid sequence that is at least 70% similar in chemical nature (such as polar or hydrophobic), if not identical, to the amino acid sequence of a naturally occurring APOBEC protein, and more preferably, at least about 75%, and more preferably, at least about 80%, and more preferably, at least about 85%, and more preferably, at least about 90%, and more preferably, at least about 95% identical to the amino acid sequence of a naturally occurring APOBEC protein. Preferred three-dimensional structural homologues of an APOBEC protein are described in detail below.

According to the present disclosure, an APOBEC “homologue”, or a “homologous” APOBEC protein, preferably has, at a minimum, one or two cytidine deamination motifs that consists of H-X-E-X₂₃₋₂₈-P-C-X₂₋₄-C (H=Histidine; X=any amino acid; E=Glutamic Acid; P=Proline; and C=Cysteine).

In general, the biological activity or biological action of a protein refers to any function(s) exhibited or performed by the protein that is ascribed to the naturally occurring form of the protein as measured or observed in vivo (i.e., in the natural physiological environment of the protein) or in vitro (i.e., under laboratory conditions). Modifications of a protein, such as in a homologue or mimetic (discussed below), may result in proteins having the same biological activity as the naturally occurring protein, or in proteins having decreased or increased biological activity as compared to the naturally occurring protein. Modifications which result in a decrease in protein expression or a decrease in the activity of the protein, can be referred to as inactivation (complete or partial), down-regulation, or decreased action of a protein. Similarly, modifications which result in an increase in protein expression or an increase in the activity of the protein can be referred to as amplification, overproduction, activation, enhancement, up-regulation or increased action of a protein. As used herein, a protein that has “biological activity” refers to a protein that has an activity that can include any one, and preferably more than one, of the following characteristics: (a) binds to the following APOBEC substrates: DNA, RNA or zinc; (b) deaminates cytosines to uracils in single-stranded DNA or RNA.

An isolated protein, according to the present disclosure, is a protein that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. Preferably, an isolated protein, and particularly, an isolated APOBEC protein, is produced recombinantly.

Proteins of the present disclosure are preferably retrieved, obtained, and/or used in “substantially pure” form. As used herein, “substantially pure” refers to a purity that allows for the effective use of the protein in vitro, ex vivo or in vivo according to the present disclosure. For a protein to be useful in an in vitro, ex vivo or in vivo method according to the present disclosure, it is substantially free of contaminants, other proteins and/or chemicals that might interfere or that would interfere with its use in a method disclosed by the present disclosure, or that at least would be undesirable for inclusion with the protein when it is used in a method disclosed by the present disclosure. Preferably, a “substantially pure” protein, as referenced herein, is a protein that can be produced by any method (i.e., by direct purification from a natural source, recombinantly, or synthetically), and that has been purified from other protein components such that the protein comprises at least about 80% weight/weight of the total protein in a given composition (i.e., the protein is about 80% of the protein in a solution/composition/buffer), and more preferably, at least about 85%, and more preferably at least about 90%, and more preferably at least about 91%, and more preferably at least about 92%, and more preferably at least about 93%, and more preferably at least about 94%, and more preferably at least about 95%, and more preferably at least about 96%, and more preferably at least about 97%, and more preferably at least about 98%, and more preferably at least about 99%, weight/weight of the total protein in a given composition.

As used herein, a “structure” of a protein refers to the components and the manner of arrangement of the components to constitute the protein. The “three dimensional structure” or “tertiary structure” of the protein refers to the arrangement of the components of the protein in three dimensions. Such term is well known to those of skill in the art. It is also to be noted that the terms “tertiary” and “three dimensional” can be used interchangeably.

As used herein, the terms “crystalline Apo3G-CD2”, “Apo3G-CD2 crystal”, “APOBEC crystal” refer to crystallized Apo3G-CD2 or APOBEC protein and are intended to be used interchangeably. Preferably, a crystalline APOBEC is produced using the crystal formation method described herein, in particular according to the method disclosed in Example 1. An Apo3G-CD2 crystal of the present disclosure can comprise any crystal structure and preferably crystallizes as an orthorhombic crystal lattice. A suitable crystalline Apo3G-CD2 of the present disclosure includes a monomer of Apo3G-CD2 protein. One preferred crystalline Apo3G-CD2 comprises one Apo3G-CD2 protein in an asymmetric unit. Preferably, a composition of the present disclosure includes Apo3G-CD2 protein molecules arranged in a crystalline manner in a space group C2 so as to form a unit cell of dimensions a=83.464 Å, b=57.329 Å, c=40.5787 Å and α=90°, β=96.46°, γ=90°. A preferred crystal of the present disclosure provides X-ray diffraction data for determination of atomic coordinates of the Apo3G-CD2 protein to a resolution of about 4.0 Å, and preferably to about 3.0 Å, and more preferably to about 2.0 Å.

As used herein, the term “model” refers to a representation in a tangible medium of the three dimensional structure of a protein, polypeptide or peptide. For example, a model can be a representation of the three dimensional structure in an electronic file, on a computer screen, on a piece of paper (i.e., on a two dimensional medium), and/or as a ball-and-stick figure. Physical three-dimensional models are tangible and include, but are not limited to, stick models and space-filling models. The phrase “imaging the model on a computer screen” refers to the ability to express (or represent) and manipulate the model on a computer screen using appropriate computer hardware and software technology known to those skilled in the art. Such technology is available from a variety of sources including, for example, Evans and Sutherland, Salt Lake City, Utah, and Biosym Technologies, San Diego, Calif. The phrase “providing a picture of the model” refers to the ability to generate a “hard copy” of the model. Hard copies include both motion and still pictures. Computer screen images and pictures of the model can be visualized in a number of formats including space-filling representations, a carbon traces, ribbon diagrams and electron density maps.

As used herein, the phrase “common amino acid side chains” refers to amino acid side chains that are common to both the structural homologue and to the structure that is actually represented by such atomic coordinates.

According to the present disclosure, the phrase “providing a three dimensional structure of APOBEC protein” is defined as any means of providing, supplying, accessing, displaying, retrieving, or otherwise making available the three dimensional structure of Apo3G-CD2 or a three dimensional computer generated structure model of an APOBEC protein. For example, the step of providing can include, but is not limited to, accessing the atomic coordinates for the structure from a database; importing the atomic coordinates for the structure into a computer or other database; displaying the atomic coordinates and/or a model of the structure in any manner, such as on a computer, on paper, etc.; and determining the three dimensional structure of Apo3G-CD2 de novo using the guidance provided herein.

As used herein, structure based drug design refers to the prediction of a conformation of a peptide, polypeptide, protein, or conformational of an interaction between a peptide or polypeptide, and a compound, using the three dimensional structure of the peptide, polypeptide or protein. Typically, structure based drug design is performed with a computer. For example, generally, for a protein to effectively interact with (or bind to) a compound, it is necessary that the three dimensional structure of the compound assume a compatible conformation that allows the compound to bind to the protein in such a manner that a desired result is obtained upon binding.

### DRAWINGS

The above-mentioned features and objects of the present disclosure will become more apparent with reference to the following description taken in conjunction with the accompanying drawings wherein like reference numerals denote like elements and in which:

FIG. 1 is the X-ray structure of the enzymatically active A3G-CD2.

FIG. 1A is a denaturing PAGE analysis of the deamination activity for full length (FL) Apo3G (lane 2) and Apo3G-CD2 (lanes 3, 4) on a fluorescein (F)-labeled ssDNA. The 32-nucleotide (nt) band shows deamination activity. As a control, no Apo3G or Apo3G-CD2 enzyme was added in lane 1.

FIG. 1B is Apo3G processivity and 3′_{—}5′ deamination bias was characterized on an 85-nt internally F-labeled ssDNA with two CCC motifs 30-nt apart. Single deaminations of the 5′C and 3′C that are spaced by 30-nt on the ssDNA substrate are detected as the appearance of labeled 67- and 48-nt fragments, respectively; double deamination of both Cs on the same molecule results in 30-nt labeled fragment (5′C and 3′C). Substrate usage (%) is less than 15% to maintain single-hit kinetics. The ‘Processivity factor’ is defined as the ratio of the observed fraction of double deaminations (occurring at both 5′C and 3′C on the same molecule) to the predicted fraction of independent double deaminations (Chelico et al., 2006). A Processivity factor greater than 1 indicates that a majority of double deaminations are caused by the same Apo3G molecule acting processively on both C targets. The deamination bias is measured by the ratio of 5′C/3′C deaminations. Deamination patterns are shown using full length Apo3G (lane 2) and Apo3G-CD2 (lanes 3, 4). As a control, no Apo3G or Apo3G-CD2 enzyme was added in lane 1.

FIGS. 1C and 1D are two views of the Apo3G-CD2 domain rotated 90° showing the 5-stranded β-sheet core surrounded by 6 helices and the extended loops around the active site. The Zn is represented as a red sphere.

FIG. 2 is the structural features of Zn-deaminase enzymes. Monomer and oligomer (insets) X-ray structures of various deaminases showing a common β-sheet core composed of five β-strands among the Zn-dependent deaminase superfamily. The active site Zn is represented by red sphere.

FIG. 2A is the Apo3G-CD2 monomer.

FIG. 2B is the Apo2 monomer; inset—an Apo2 tetramer (PDB 2nyt). The elongated Apo2 tetramer is formed from a head-head interaction between two dimers (h4 and h6 from each dimer are labeled). Each Apo2 dimer is formed through the pairing of 132 strands from each monomer (132 strands are labeled in left dimer).

FIG. 2C is the Staphylococcus aureus tRNA adenosine deaminase TadA monomer; inset—a TadA dimer (PDB 2b3j). (D) The human free-nucleotide cytidine deaminase (hCDA) monomer; inset—a square shaped hCDA tetramer (PDB imqo).

FIG. 2E is the ScCDD1 monomer; inset—a square shaped CDD1 tetramer (PDB irst).

FIG. 2F is the E. coli free-nt cytidine deaminase monomer; inset—a square-shaped ECDA dimer (PDB 1ALN).

FIG. 3 is structural comparison of Apo3G-CD2 with Apo2 and of their active center loop (AC-loop) conformations.

FIG. 3A shows core structural elements of Apo3G-CD2 (yellow) and Apo2 (cyan) superimposed with flexible loops removed. Red sphere represents Zn.

FIG. 3B is the superposition of Apo3G-CD2 and an Apo2 monomer containing AC-Loop 1 conformation I where the loop is collapsed over the active site.

FIG. 3C is the superposition of Apo3G-CD2 and an Apo2 monomer containing AC-Loop 1 conformation II where the loop forms a a-hairpin and is pulled back from the active site.

FIG. 3D shows that AC-Loop 1 is stabilized by hydrogen bonds (green dashed lines) between residues R215 and F204, E211, N207, E209, W285 (pink), as well as by hydrophobic packing between the aliphatic chain of R215 with F204, R313 and W285. The interactions of R215 with R313 and W285 should also helps to stabilize the local conformation near the active site.

FIG. 3E shows that AC-Loop 3 is stabilized by a network of main chain hydrogen bond interactions (green dashed line) between R256, F252, G251, H248 and G244 (pink). N244 (cyan) is highly conserved sequence-wise and structurally near the active site among diverse Zn-deaminases. The equivalent N244 is shown to contact the target base (cyan) in TadA and hCDA (Chung et al., 2005; Losey et al., 2006). Zn atom (red sphere) is coordinated by active site residues H257, C288, and C291 (wheat).

FIG. 4 is a structural comparison of the Apo3G-CD2 X-ray structure with the Apo3G-2K3A NMR structure.

FIG. 4A is the superposition of the Apo3G-CD2 X-ray structure (yellow) and the Apo3G-2K3A NMR structure (gray) (RMSD=4.8 Å²). The residues which form the β2 strand in the X-ray structure form a loop-like bulge in the NMR structure (thickened loop). Inset—superposition of Apo3G-CD2 (yellow) and an Apo2 monomer (cyan) (RMSD=2.7 Å²).

FIGS. 4B and 4C show two views of the superposition of the Apo3G-CD2 X-ray structure (yellow) and Apo3G-2K3A NMR structure (gray) with helices 2, 3, and 4 removed to show the differences in h1, β2, AC-loop 1, and AC-loop 3. The view in panel C is rotated 180° relative to that in panel B. Highlighted are two of the five point mutations, L234K and C243A, that were made in order to obtain soluble protein for the Apo3G-2K3A NMR structure. These mutations are located on the N and C-terminus of the β2 strand of the X-ray structure (blue), and on the loop-like bulge of the NMR structure (green).

FIG. 5 shows residues important for deamination activity and ssDNA substrate binding.

FIG. 5A is the active site of A3G-CD2 shows Zn (red sphere) coordinated by H257, C288, C291, and a water molecule at a hydrogen bond distance of 2.5 Å (cyan sphere). The E259 below the Zn is important for proton shuffling to facilitate the Zn atom to deaminate the target base that approach the Zn from the direction of the water molecule.

FIG. 5B (left) is the superposition of Apo3G-CD2 (yellow) and TadA (light blue, PDB 2b3j). The TadA residues, H53 and N42 (blue), that contact the TadA substrate (green) overlap well with the corresponding conserved residues, H257 and N244, on the AC-loop 3 of Apo3G-CD2.

FIG. 5B (right) is the superposition of Apo3G-CD2 (yellow) and hCDA (pink, hCDA), showing hCDA residues, C65 and N54, (magenta) that contact the hCDA substrate analog diazepinone riboside (green) overlap well with H257 and N244 of Apo3G-CD2 (sand).

FIG. 5C shows positive residues R213, R256, R320, R374 and R376 located around the active center. Residues H247, W285, Y315, and F289 near the active site could potentially interact with incoming ssDNA via hydrophobic base stacking to orient substrate for deamination.

FIG. 5D is a surface representation showing the pocket (or groove) around the active site, with the positive residues (colored in blue) lining the periphery, and the hydrophobic residues (colored in yellow) near the active Zn atom (red sphere). (E, left) Mutational data from Sf9 purified full-length wt and mutant Apo3G. Black bars represent deamination results and dark blue bars represent ssDNA binding results.

FIG. 5E (right) is mutational data from E. coli purified full-length wild-type and mutant Apo3G. Black bars represent deamination results.

FIG. 5E (right inset) is relative deamination of the last 3′C (5′CCC) or the middle C (5′CCC) on the 5′CCC motif of a ssDNA substrate.

FIG. 6 is a potential ssDNA binding groove for Apo3G-CD2. All panels are shown in the same orientation as used previously to describe the DNA binding model in Chen, et al., 2008.

FIG. 6A is the X-ray structure of Apo3G-CD2 with residues predicted to interact with ssDNA (shown as sticks in magenta).

FIG. 6B is a surface representation of the X-ray structure of Apo3G-CD2, showing a horizontal groove with residues predicted to interact with ssDNA lining around the groove (shown as sticks in magenta). Mutational analysis of most of these residues has demonstrated their important role in deaminase activity (FIG. 5E). The Apo3G-CD2 AC-loop 1, AC-loop 3 and helix 1 (yellow) provide a wide open groove that may be used for DNA to bind. Predicted ssDNA binding is represented by a green line along the “substrate” groove, with the target cytidine (in green) presented to the active site Zn atom from the only accessible direction for deamination.

FIG. 6C is a surface representation of the X-ray structure of Apo3G-CD2 with the NMR AC-loop (line in dark blue) blocking the groove formed between AC-loop 1 and 3 in the X-ray structure (in yellow).

FIG. 6D is the NMR structure of Apo3G-2K3A with residues previously predicted to interact with ssDNA (sticks in dark blue). The positions of some of these residues on the X-ray structure are shown in 6A, and the positions of all these residues on the X-ray structure are shown in FIG. 5C.

FIG. 6E is a surface representation of NMR structure of Apo3G-2K3A shown in the same orientation as in 6D with residues predicted to interact with ssDNA (sticks in dark blue). Predicted ssDNA binding is represented by a green dashed line.

FIG. 6F is a surface representation of NMR structure of Apo3G-2K3A with the X-ray Apo3G-CD2 helix 1 shown to block the ssDNA-binding path in the previously proposed model.

FIG. 7 is the model of a full length Apo3G molecule.

FIG. 7A is a model for the full length Apo3G monomer. The Apo3G-CD1 (violet) is modeled using the structures of Apo3G-CD2 (yellow) and the Apo2 Loop 3. The Apo3G-CD1 and CD2 domain interface through the β2 strands is modeled using the Apo2 dimer as a template. The Apo3G-CD1 residues that are aligned well with the Apo2 tetramerization residues (residues indicated by green dots in the sequence alignment in Supplementary Figure) are predicted to form the dimeric interface in an Apo3G head-head dimer. These residues have also been shown to be important in virion incorporation and HIV-1 viral restriction. The active site Zn is represented by a red sphere.

FIG. 7B is a model for a head-head (or N—N) dimer of Apo3G joining through CD1-CD1 (violet) interactions using the Apo2 tetramer as a structural template. Helix 4 and 6 are labeled and, as seen in Apo2, may be important for elongated oligomer interaction. Residue D128 is important for species specific recognition of Apo3G by the HIV-1 VIF protein.

FIG. 7C is a model for a head-tail dimer of Apo3G joining through CD1 (violet) and CD2 (yellow) interactions.

TABLE 1
Apo3G-CD2 (APOBEC-3G-CD2) Monomer

| | | | | | | | | | | | |
|---|---|---|---|---|---|---|---|---|---|---|---|
| ATOM | 1 | N | MET | A | 197 | 18.313 | 44.759 | 13.063 | 1.00 | 26.43 | N |
| ATOM | 2 | CA | MET | A | 197 | 16.859 | 44.439 | 13.208 | 1.00 | 26.60 | C |
| ATOM | 3 | C | MET | A | 197 | 16.364 | 44.988 | 14.550 | 1.00 | 28.07 | C |
| ATOM | 4 | O | MET | A | 197 | 16.962 | 44.723 | 15.587 | 1.00 | 29.18 | O |
| ATOM | 5 | CB | MET | A | 197 | 16.653 | 42.924 | 13.147 | 1.00 | 24.03 | C |
| ATOM | 6 | CG | MET | A | 197 | 15.191 | 42.484 | 12.991 | 1.00 | 22.95 | C |
| ATOM | 7 | SD | MET | A | 197 | 14.937 | 40.676 | 13.025 | 1.00 | 18.54 | S |
| ATOM | 8 | CE | MET | A | 197 | 16.335 | 40.141 | 12.054 | 1.00 | 14.37 | C |
| ATOM | 9 | N | ASP | A | 198 | 15.277 | 45.753 | 14.533 | 1.00 | 29.10 | N |
| ATOM | 10 | CA | ASP | A | 198 | 14.751 | 46.310 | 15.772 | 1.00 | 31.49 | C |
| ATOM | 11 | C | ASP | A | 198 | 14.107 | 45.210 | 16.618 | 1.00 | 29.72 | C |
| ATOM | 12 | O | ASP | A | 198 | 13.505 | 44.280 | 16.088 | 1.00 | 29.97 | O |
| ATOM | 13 | CB | ASP | A | 198 | 13.733 | 47.416 | 15.476 | 1.00 | 34.76 | C |
| ATOM | 14 | CG | ASP | A | 198 | 12.529 | 46.909 | 14.718 | 1.00 | 38.13 | C |
| ATOM | 15 | OD1 | ASP | A | 198 | 12.698 | 46.460 | 13.557 | 1.00 | 39.02 | O |
| ATOM | 16 | OD2 | ASP | A | 198 | 11.415 | 46.957 | 15.289 | 1.00 | 38.75 | O |
| ATOM | 17 | N | PRO | A | 199 | 14.237 | 45.310 | 17.950 | 1.00 | 28.25 | N |
| ATOM | 18 | CA | PRO | A | 199 | 13.696 | 44.355 | 18.925 | 1.00 | 27.03 | C |
| ATOM | 19 | C | PRO | A | 199 | 12.253 | 43.890 | 18.702 | 1.00 | 25.77 | C |
| ATOM | 20 | O | PRO | A | 199 | 11.975 | 42.692 | 18.701 | 1.00 | 23.34 | O |
| ATOM | 21 | CB | PRO | A | 199 | 13.887 | 45.085 | 20.251 | 1.00 | 27.43 | C |
| ATOM | 22 | CG | PRO | A | 199 | 15.188 | 45.809 | 20.013 | 1.00 | 28.32 | C |
| ATOM | 23 | CD | PRO | A | 199 | 14.976 | 46.389 | 18.633 | 1.00 | 27.68 | C |
| ATOM | 24 | N | PRO | A | 200 | 11.317 | 44.834 | 18.513 | 1.00 | 25.94 | N |
| ATOM | 25 | CA | PRO | A | 200 | 9.916 | 44.444 | 18.292 | 1.00 | 25.11 | C |
| ATOM | 26 | C | PRO | A | 200 | 9.761 | 43.487 | 17.104 | 1.00 | 24.17 | C |
| ATOM | 27 | O | PRO | A | 200 | 9.048 | 42.494 | 17.188 | 1.00 | 26.17 | O |
| ATOM | 28 | CB | PRO | A | 200 | 9.212 | 45.786 | 18.041 | 1.00 | 25.32 | C |
| ATOM | 29 | CG | PRO | A | 200 | 10.069 | 46.778 | 18.805 | 1.00 | 23.67 | C |
| ATOM | 30 | CD | PRO | A | 200 | 11.473 | 46.302 | 18.494 | 1.00 | 25.39 | C |
| ATOM | 31 | N | THR | A | 201 | 10.436 | 43.787 | 16.003 | 1.00 | 23.73 | N |
| ATOM | 32 | CA | THR | A | 201 | 10.357 | 42.954 | 14.807 | 1.00 | 24.32 | C |
| ATOM | 33 | C | THR | A | 201 | 10.868 | 41.538 | 15.066 | 1.00 | 25.64 | C |
| ATOM | 34 | O | THR | A | 201 | 10.210 | 40.558 | 14.718 | 1.00 | 25.06 | O |
| ATOM | 35 | CB | THR | A | 201 | 11.145 | 43.612 | 13.665 | 1.00 | 23.15 | C |
| ATOM | 36 | CG2 | THR | A | 201 | 11.040 | 42.789 | 12.371 | 1.00 | 21.95 | C |
| ATOM | 37 | OG1 | THR | A | 201 | 10.602 | 44.924 | 13.445 | 1.00 | 23.05 | O |
| ATOM | 38 | N | PHE | A | 202 | 12.036 | 41.441 | 15.697 | 1.00 | 26.74 | N |
| ATOM | 39 | CA | PHE | A | 202 | 12.626 | 40.151 | 16.026 | 1.00 | 25.32 | C |
| ATOM | 40 | C | PHE | A | 202 | 11.661 | 39.362 | 16.886 | 1.00 | 26.36 | C |
| ATOM | 41 | O | PHE | A | 202 | 11.258 | 38.257 | 16.531 | 1.00 | 27.72 | O |
| ATOM | 42 | CB | PHE | A | 202 | 13.937 | 40.335 | 16.796 | 1.00 | 24.02 | C |
| ATOM | 43 | CG | PHE | A | 202 | 14.495 | 39.052 | 17.360 | 1.00 | 23.02 | C |
| ATOM | 44 | CD1 | PHE | A | 202 | 15.033 | 38.085 | 16.521 | 1.00 | 24.56 | C |
| ATOM | 45 | CD2 | PHE | A | 202 | 14.424 | 38.786 | 18.718 | 1.00 | 23.13 | C |
| ATOM | 46 | CE1 | PHE | A | 202 | 15.495 | 36.864 | 17.028 | 1.00 | 23.28 | C |
| ATOM | 47 | CE2 | PHE | A | 202 | 14.880 | 37.575 | 19.237 | 1.00 | 25.22 | C |
| ATOM | 48 | CZ | PHE | A | 202 | 15.415 | 36.610 | 18.382 | 1.00 | 23.39 | C |
| ATOM | 49 | N | THR | A | 203 | 11.286 | 39.943 | 18.021 | 1.00 | 28.60 | N |
| ATOM | 50 | CA | THR | A | 203 | 10.391 | 39.272 | 18.960 | 1.00 | 28.49 | C |
| ATOM | 51 | C | THR | A | 203 | 9.069 | 38.867 | 18.329 | 1.00 | 26.94 | C |
| ATOM | 52 | O | THR | A | 203 | 8.543 | 37.791 | 18.610 | 1.00 | 25.95 | O |
| ATOM | 53 | CB | THR | A | 203 | 10.095 | 40.154 | 20.189 | 1.00 | 30.96 | C |
| ATOM | 54 | CG2 | THR | A | 203 | 9.550 | 39.294 | 21.326 | 1.00 | 32.18 | C |
| ATOM | 55 | OG1 | THR | A | 203 | 11.307 | 40.769 | 20.639 | 1.00 | 34.82 | O |
| ATOM | 56 | N | PHE | A | 204 | 8.526 | 39.725 | 17.477 | 1.00 | 25.34 | N |
| ATOM | 57 | CA | PHE | A | 204 | 7.260 | 39.395 | 16.837 | 1.00 | 24.66 | C |
| ATOM | 58 | C | PHE | A | 204 | 7.416 | 38.323 | 15.757 | 1.00 | 23.07 | C |
| ATOM | 59 | O | PHE | A | 204 | 6.556 | 37.457 | 15.602 | 1.00 | 23.98 | O |
| ATOM | 60 | CB | PHE | A | 204 | 6.621 | 40.637 | 16.219 | 1.00 | 24.12 | C |
| ATOM | 61 | CG | PHE | A | 204 | 5.417 | 40.333 | 15.370 | 1.00 | 26.63 | C |
| ATOM | 62 | CD1 | PHE | A | 204 | 4.228 | 39.894 | 15.949 | 1.00 | 25.72 | C |
| ATOM | 63 | CD2 | PHE | A | 204 | 5.483 | 40.461 | 13.983 | 1.00 | 26.42 | C |
| ATOM | 64 | CE1 | PHE | A | 204 | 3.118 | 39.582 | 15.158 | 1.00 | 27.50 | C |
| ATOM | 65 | CE2 | PHE | A | 204 | 4.378 | 40.152 | 13.181 | 1.00 | 28.18 | C |
| ATOM | 66 | CZ | PHE | A | 204 | 3.191 | 39.710 | 13.770 | 1.00 | 26.14 | C |
| ATOM | 67 | N | ASN | A | 205 | 8.514 | 38.373 | 15.013 | 1.00 | 22.62 | N |
| ATOM | 68 | CA | ASN | A | 205 | 8.707 | 37.411 | 13.944 | 1.00 | 21.07 | C |
| ATOM | 69 | C | ASN | A | 205 | 9.290 | 36.059 | 14.335 | 1.00 | 23.66 | C |
| ATOM | 70 | O | ASN | A | 205 | 8.966 | 35.037 | 13.711 | 1.00 | 22.51 | O |
| ATOM | 71 | CB | ASN | A | 205 | 9.544 | 38.035 | 12.831 | 1.00 | 21.40 | C |
| ATOM | 72 | CG | ASN | A | 205 | 8.762 | 39.081 | 12.025 | 1.00 | 22.36 | C |
| ATOM | 73 | ND2 | ASN | A | 205 | 8.095 | 38.629 | 10.978 | 1.00 | 19.24 | N |
| ATOM | 74 | OD1 | ASN | A | 205 | 8.758 | 40.274 | 12.349 | 1.00 | 20.85 | O |
| ATOM | 75 | N | PHE | A | 206 | 10.137 | 36.039 | 15.363 | 1.00 | 23.26 | N |
| ATOM | 76 | CA | PHE | A | 206 | 10.772 | 34.793 | 15.794 | 1.00 | 22.81 | C |
| ATOM | 77 | C | PHE | A | 206 | 10.119 | 34.108 | 16.994 | 1.00 | 23.94 | C |
| ATOM | 78 | O | PHE | A | 206 | 10.600 | 33.069 | 17.462 | 1.00 | 21.97 | O |
| ATOM | 79 | CB | PHE | A | 206 | 12.258 | 35.037 | 16.075 | 1.00 | 21.69 | C |
| ATOM | 80 | CG | PHE | A | 206 | 13.091 | 35.208 | 14.832 | 1.00 | 19.37 | C |
| ATOM | 81 | CD1 | PHE | A | 206 | 13.172 | 36.442 | 14.186 | 1.00 | 19.18 | C |
| ATOM | 82 | CD2 | PHE | A | 206 | 13.775 | 34.126 | 14.293 | 1.00 | 19.22 | C |
| ATOM | 83 | CE1 | PHE | A | 206 | 13.919 | 36.592 | 13.024 | 1.00 | 14.86 | C |
| ATOM | 84 | CE2 | PHE | A | 206 | 14.527 | 34.259 | 13.130 | 1.00 | 16.90 | C |
| ATOM | 85 | CZ | PHE | A | 206 | 14.597 | 35.502 | 12.493 | 1.00 | 19.54 | C |
| ATOM | 86 | N | ASN | A | 207 | 9.034 | 34.691 | 17.498 | 1.00 | 25.07 | N |
| ATOM | 87 | CA | ASN | A | 207 | 8.312 | 34.094 | 18.620 | 1.00 | 26.86 | C |
| ATOM | 88 | C | ASN | A | 207 | 7.841 | 32.732 | 18.113 | 1.00 | 27.30 | C |
| ATOM | 89 | O | ASN | A | 207 | 7.286 | 32.634 | 17.012 | 1.00 | 28.11 | O |
| ATOM | 90 | CB | ASN | A | 207 | 7.127 | 34.990 | 19.007 | 1.00 | 27.48 | C |
| ATOM | 91 | CG | ASN | A | 207 | 6.184 | 34.331 | 19.996 | 1.00 | 29.49 | C |
| ATOM | 92 | ND2 | ASN | A | 207 | 6.136 | 34.865 | 21.211 | 1.00 | 34.31 | N |
| ATOM | 93 | OD1 | ASN | A | 207 | 5.508 | 33.360 | 19.676 | 1.00 | 29.10 | O |
| ATOM | 94 | N | ASN | A | 208 | 8.072 | 31.673 | 18.886 | 1.00 | 28.66 | N |
| ATOM | 95 | CA | ASN | A | 208 | 7.675 | 30.344 | 18.421 | 1.00 | 30.76 | C |
| ATOM | 96 | C | ASN | A | 208 | 6.330 | 29.825 | 18.897 | 1.00 | 36.62 | C |
| ATOM | 97 | O | ASN | A | 208 | 6.132 | 28.615 | 19.006 | 1.00 | 35.18 | O |
| ATOM | 98 | CB | ASN | A | 208 | 8.777 | 29.300 | 18.697 | 1.00 | 26.26 | C |
| ATOM | 99 | CG | ASN | A | 208 | 9.112 | 29.133 | 20.181 | 1.00 | 24.78 | C |
| ATOM | 100 | ND2 | ASN | A | 208 | 10.140 | 28.331 | 20.457 | 1.00 | 19.91 | N |
| ATOM | 101 | OD1 | ASN | A | 208 | 8.457 | 29.690 | 21.060 | 1.00 | 23.25 | O |
| ATOM | 102 | N | GLU | A | 209 | 5.398 | 30.742 | 19.153 | 1.00 | 43.86 | N |
| ATOM | 103 | CA | GLU | A | 209 | 4.057 | 30.360 | 19.578 | 1.00 | 52.45 | C |
| ATOM | 104 | C | GLU | A | 209 | 3.261 | 29.977 | 18.336 | 1.00 | 58.10 | C |
| ATOM | 105 | O | GLU | A | 209 | 2.820 | 30.841 | 17.582 | 1.00 | 58.90 | O |
| ATOM | 106 | CB | GLU | A | 209 | 3.368 | 31.510 | 20.311 | 1.00 | 52.56 | C |
| ATOM | 107 | CG | GLU | A | 209 | 4.041 | 31.877 | 21.620 | 1.00 | 53.68 | C |
| ATOM | 108 | CD | GLU | A | 209 | 3.310 | 32.978 | 22.358 | 1.00 | 55.01 | C |
| ATOM | 109 | OE1 | GLU | A | 209 | 2.976 | 33.997 | 21.717 | 1.00 | 54.06 | O |
| ATOM | 110 | OE2 | GLU | A | 209 | 3.079 | 32.829 | 23.577 | 1.00 | 54.19 | O |
| ATOM | 111 | N | PRO | A | 210 | 3.057 | 28.667 | 18.125 | 1.00 | 64.07 | N |
| ATOM | 112 | CA | PRO | A | 210 | 2.338 | 28.028 | 17.014 | 1.00 | 68.69 | C |
| ATOM | 113 | C | PRO | A | 210 | 1.579 | 28.959 | 16.061 | 1.00 | 72.74 | C |
| ATOM | 114 | O | PRO | A | 210 | 1.918 | 29.051 | 14.880 | 1.00 | 72.44 | O |
| ATOM | 115 | CB | PRO | A | 210 | 1.427 | 27.050 | 17.740 | 1.00 | 68.73 | C |
| ATOM | 116 | CG | PRO | A | 210 | 2.352 | 26.528 | 18.793 | 1.00 | 67.97 | C |
| ATOM | 117 | CD | PRO | A | 210 | 3.086 | 27.766 | 19.294 | 1.00 | 65.92 | C |
| ATOM | 118 | N | TRP | A | 211 | 0.555 | 29.629 | 16.590 | 1.00 | 77.20 | N |
| ATOM | 119 | CA | TRP | A | 211 | −0.293 | 30.572 | 15.853 | 1.00 | 81.49 | C |
| ATOM | 120 | C | TRP | A | 211 | 0.339 | 31.171 | 14.585 | 1.00 | 81.22 | C |
| ATOM | 121 | O | TRP | A | 211 | 1.463 | 31.668 | 14.611 | 1.00 | 81.21 | O |
| ATOM | 122 | CB | TRP | A | 211 | −0.665 | 31.759 | 16.755 | 1.00 | 87.61 | C |
| ATOM | 123 | CG | TRP | A | 211 | −2.127 | 32.194 | 16.884 | 1.00 | 94.05 | C |
| ATOM | 124 | CD1 | TRP | A | 211 | −2.598 | 33.186 | 17.716 | 1.00 | 95.79 | C |
| ATOM | 125 | CD2 | TRP | A | 211 | −3.279 | 31.680 | 16.189 | 1.00 | 97.37 | C |
| ATOM | 126 | CE2 | TRP | A | 211 | −4.405 | 32.407 | 16.659 | 1.00 | 98.68 | C |
| ATOM | 127 | CE3 | TRP | A | 211 | −3.475 | 30.676 | 15.223 | 1.00 | 99.06 | C |
| ATOM | 128 | NE1 | TRP | A | 211 | −3.958 | 33.315 | 17.583 | 1.00 | 97.88 | N |
| ATOM | 129 | CZ2 | TRP | A | 211 | −5.702 | 32.165 | 16.193 | 1.00 | 100.00 | C |
| ATOM | 130 | CZ3 | TRP | A | 211 | −4.773 | 30.437 | 14.762 | 1.00 | 100.26 | C |
| ATOM | 131 | CH2 | TRP | A | 211 | −5.865 | 31.178 | 15.251 | 1.00 | 100.56 | C |
| ATOM | 132 | N | VAL | A | 212 | −0.382 | 31.126 | 13.475 | 1.00 | 80.22 | N |
| ATOM | 133 | CA | VAL | A | 212 | 0.098 | 31.754 | 12.255 | 1.00 | 79.38 | C |
| ATOM | 134 | C | VAL | A | 212 | −0.875 | 32.923 | 12.167 | 1.00 | 78.28 | C |
| ATOM | 135 | O | VAL | A | 212 | −1.760 | 32.958 | 11.317 | 1.00 | 78.75 | O |
| ATOM | 136 | CB | VAL | A | 212 | −0.072 | 30.859 | 11.011 | 1.00 | 80.01 | C |
| ATOM | 137 | CG1 | VAL | A | 212 | 0.502 | 31.569 | 9.788 | 1.00 | 80.27 | C |
| ATOM | 138 | CG2 | VAL | A | 212 | 0.620 | 29.524 | 11.234 | 1.00 | 80.59 | C |
| ATOM | 139 | N | ARG | A | 213 | −0.714 | 33.873 | 13.080 | 1.00 | 75.66 | N |
| ATOM | 140 | CA | ARG | A | 213 | −1.604 | 35.025 | 13.159 | 1.00 | 72.27 | C |
| ATOM | 141 | C | ARG | A | 213 | −0.830 | 36.337 | 13.149 | 1.00 | 68.55 | C |
| ATOM | 142 | O | ARG | A | 213 | −0.441 | 36.845 | 14.194 | 1.00 | 69.01 | O |
| ATOM | 143 | CB | ARG | A | 213 | −2.462 | 34.889 | 14.434 | 1.00 | 73.90 | C |
| ATOM | 144 | CG | ARG | A | 213 | −3.673 | 35.805 | 14.529 | 1.00 | 75.17 | C |
| ATOM | 145 | CD | ARG | A | 213 | −3.321 | 37.143 | 15.152 | 1.00 | 76.17 | C |
| ATOM | 146 | NE | ARG | A | 213 | −2.976 | 37.019 | 16.568 | 1.00 | 77.40 | N |
| ATOM | 147 | CZ | ARG | A | 213 | −2.634 | 38.045 | 17.341 | 1.00 | 78.35 | C |
| ATOM | 148 | NH1 | ARG | A | 213 | −2.588 | 39.270 | 16.833 | 1.00 | 78.62 | N |
| ATOM | 149 | NH2 | ARG | A | 213 | −2.346 | 37.848 | 18.622 | 1.00 | 78.63 | N |
| ATOM | 150 | N | GLY | A | 214 | −0.599 | 36.872 | 11.955 | 1.00 | 63.75 | N |
| ATOM | 151 | CA | GLY | A | 214 | 0.127 | 38.118 | 11.843 | 1.00 | 57.72 | C |
| ATOM | 152 | C | GLY | A | 214 | 1.467 | 37.969 | 11.154 | 1.00 | 53.14 | C |
| ATOM | 153 | O | GLY | A | 214 | 1.832 | 38.807 | 10.334 | 1.00 | 51.65 | O |
| ATOM | 154 | N | ARG | A | 215 | 2.206 | 36.912 | 11.477 | 1.00 | 49.82 | N |
| ATOM | 155 | CA | ARG | A | 215 | 3.512 | 36.705 | 10.861 | 1.00 | 46.50 | C |
| ATOM | 156 | C | ARG | A | 215 | 3.368 | 36.241 | 9.419 | 1.00 | 44.46 | C |
| ATOM | 157 | O | ARG | A | 215 | 3.462 | 35.049 | 9.127 | 1.00 | 41.76 | O |
| ATOM | 158 | CB | ARG | A | 215 | 4.344 | 35.692 | 11.664 | 1.00 | 45.65 | C |
| ATOM | 159 | CG | ARG | A | 215 | 4.853 | 36.211 | 13.009 | 1.00 | 45.16 | C |
| ATOM | 160 | CD | ARG | A | 215 | 3.766 | 36.210 | 14.076 | 1.00 | 44.44 | C |
| ATOM | 161 | NE | ARG | A | 215 | 3.459 | 34.858 | 14.531 | 1.00 | 45.41 | N |
| ATOM | 162 | CZ | ARG | A | 215 | 4.224 | 34.145 | 15.357 | 1.00 | 45.44 | C |
| ATOM | 163 | NH1 | ARG | A | 215 | 5.353 | 34.649 | 15.838 | 1.00 | 44.25 | N |
| ATOM | 164 | NH2 | ARG | A | 215 | 3.864 | 32.914 | 15.697 | 1.00 | 45.49 | N |
| ATOM | 165 | N | HIS | A | 216 | 3.129 | 37.196 | 8.523 | 1.00 | 43.04 | N |
| ATOM | 166 | CA | HIS | A | 216 | 2.976 | 36.880 | 7.110 | 1.00 | 42.85 | C |
| ATOM | 167 | C | HIS | A | 216 | 4.312 | 37.014 | 6.385 | 1.00 | 39.73 | C |
| ATOM | 168 | O | HIS | A | 216 | 4.404 | 36.733 | 5.198 | 1.00 | 41.31 | O |
| ATOM | 169 | CB | HIS | A | 216 | 1.929 | 37.789 | 6.443 | 1.00 | 46.04 | C |
| ATOM | 170 | CG | HIS | A | 216 | 0.595 | 37.785 | 7.124 | 1.00 | 48.11 | C |
| ATOM | 171 | CD2 | HIS | A | 216 | −0.508 | 37.024 | 6.929 | 1.00 | 49.39 | C |
| ATOM | 172 | ND1 | HIS | A | 216 | 0.293 | 38.631 | 8.171 | 1.00 | 50.15 | N |
| ATOM | 173 | CE1 | HIS | A | 216 | −0.935 | 38.390 | 8.591 | 1.00 | 51.51 | C |
| ATOM | 174 | NE2 | HIS | A | 216 | −1.444 | 37.418 | 7.855 | 1.00 | 50.61 | N |
| ATOM | 175 | N | GLU | A | 217 | 5.349 | 37.446 | 7.090 | 1.00 | 36.93 | N |
| ATOM | 176 | CA | GLU | A | 217 | 6.656 | 37.559 | 6.465 | 1.00 | 33.68 | C |
| ATOM | 177 | C | GLU | A | 217 | 7.579 | 36.473 | 6.989 | 1.00 | 30.91 | C |
| ATOM | 178 | O | GLU | A | 217 | 7.208 | 35.709 | 7.876 | 1.00 | 27.08 | O |
| ATOM | 179 | CB | GLU | A | 217 | 7.246 | 38.931 | 6.726 | 1.00 | 36.37 | C |
| ATOM | 180 | CG | GLU | A | 217 | 6.326 | 40.017 | 6.216 | 1.00 | 40.53 | C |
| ATOM | 181 | CD | GLU | A | 217 | 6.769 | 41.386 | 6.617 | 1.00 | 43.87 | C |
| ATOM | 182 | OE1 | GLU | A | 217 | 7.834 | 41.833 | 6.130 | 1.00 | 46.60 | O |
| ATOM | 183 | OE2 | GLU | A | 217 | 6.049 | 42.021 | 7.428 | 1.00 | 47.12 | O |
| ATOM | 184 | N | THR | A | 218 | 8.773 | 36.400 | 6.408 | 1.00 | 29.83 | N |
| ATOM | 185 | CA | THR | A | 218 | 9.785 | 35.419 | 6.782 | 1.00 | 25.52 | C |
| ATOM | 186 | C | THR | A | 218 | 11.139 | 36.126 | 6.891 | 1.00 | 25.88 | C |
| ATOM | 187 | O | THR | A | 218 | 11.676 | 36.574 | 5.881 | 1.00 | 27.29 | O |
| ATOM | 188 | CB | THR | A | 218 | 9.913 | 34.304 | 5.699 | 1.00 | 24.23 | C |
| ATOM | 189 | CG2 | THR | A | 218 | 11.122 | 33.426 | 5.980 | 1.00 | 21.53 | C |
| ATOM | 190 | OG1 | THR | A | 218 | 8.734 | 33.488 | 5.683 | 1.00 | 18.00 | O |
| ATOM | 191 | N | TYR | A | 219 | 11.679 | 36.256 | 8.103 | 1.00 | 22.41 | N |
| ATOM | 192 | CA | TYR | A | 219 | 12.992 | 36.882 | 8.250 | 1.00 | 20.83 | C |
| ATOM | 193 | C | TYR | A | 219 | 14.065 | 35.803 | 8.265 | 1.00 | 20.01 | C |
| ATOM | 194 | O | TYR | A | 219 | 13.862 | 34.714 | 8.796 | 1.00 | 19.03 | O |
| ATOM | 195 | CB | TYR | A | 219 | 13.082 | 37.739 | 9.525 | 1.00 | 21.40 | C |
| ATOM | 196 | CG | TYR | A | 219 | 12.414 | 39.087 | 9.367 | 1.00 | 20.31 | C |
| ATOM | 197 | CD1 | TYR | A | 219 | 11.026 | 39.190 | 9.321 | 1.00 | 19.95 | C |
| ATOM | 198 | CD2 | TYR | A | 219 | 13.167 | 40.250 | 9.205 | 1.00 | 22.36 | C |
| ATOM | 199 | CE1 | TYR | A | 219 | 10.398 | 40.416 | 9.117 | 1.00 | 21.85 | C |
| ATOM | 200 | CE2 | TYR | A | 219 | 12.545 | 41.488 | 8.999 | 1.00 | 24.06 | C |
| ATOM | 201 | CZ | TYR | A | 219 | 11.156 | 41.558 | 8.957 | 1.00 | 23.43 | C |
| ATOM | 202 | OH | TYR | A | 219 | 10.528 | 42.768 | 8.757 | 1.00 | 28.48 | O |
| ATOM | 203 | N | LEU | A | 220 | 15.199 | 36.111 | 7.656 | 1.00 | 20.05 | N |
| ATOM | 204 | CA | LEU | A | 220 | 16.297 | 35.170 | 7.571 | 1.00 | 20.08 | C |
| ATOM | 205 | C | LEU | A | 220 | 17.624 | 35.855 | 7.879 | 1.00 | 20.12 | C |
| ATOM | 206 | O | LEU | A | 220 | 18.001 | 36.826 | 7.220 | 1.00 | 18.03 | O |
| ATOM | 207 | CB | LEU | A | 220 | 16.335 | 34.554 | 6.167 | 1.00 | 19.38 | C |
| ATOM | 208 | CG | LEU | A | 220 | 17.435 | 33.544 | 5.841 | 1.00 | 23.82 | C |
| ATOM | 209 | CD1 | LEU | A | 220 | 17.008 | 32.713 | 4.626 | 1.00 | 24.01 | C |
| ATOM | 210 | CD2 | LEU | A | 220 | 18.775 | 34.264 | 5.584 | 1.00 | 18.58 | C |
| ATOM | 211 | N | CYS | A | 221 | 18.303 | 35.341 | 8.901 | 1.00 | 22.46 | N |
| ATOM | 212 | CA | CYS | A | 221 | 19.606 | 35.818 | 9.336 | 1.00 | 24.19 | C |
| ATOM | 213 | C | CYS | A | 221 | 20.611 | 34.761 | 8.882 | 1.00 | 25.21 | C |
| ATOM | 214 | O | CYS | A | 221 | 20.341 | 33.565 | 8.992 | 1.00 | 25.88 | O |
| ATOM | 215 | CB | CYS | A | 221 | 19.668 | 35.910 | 10.860 | 1.00 | 25.63 | C |
| ATOM | 216 | SG | CYS | A | 221 | 18.426 | 36.983 | 11.619 | 1.00 | 31.74 | S |
| ATOM | 217 | N | TYR | A | 222 | 21.768 | 35.196 | 8.396 | 1.00 | 25.43 | N |
| ATOM | 218 | CA | TYR | A | 222 | 22.792 | 34.268 | 7.930 | 1.00 | 25.78 | C |
| ATOM | 219 | C | TYR | A | 222 | 24.213 | 34.653 | 8.370 | 1.00 | 27.41 | C |
| ATOM | 220 | O | TYR | A | 222 | 24.522 | 35.830 | 8.618 | 1.00 | 25.56 | O |
| ATOM | 221 | CB | TYR | A | 222 | 22.723 | 34.162 | 6.400 | 1.00 | 27.29 | C |
| ATOM | 222 | CG | TYR | A | 222 | 22.930 | 35.479 | 5.688 | 1.00 | 28.11 | C |
| ATOM | 223 | CD1 | TYR | A | 222 | 24.216 | 35.945 | 5.401 | 1.00 | 30.56 | C |
| ATOM | 224 | CD2 | TYR | A | 222 | 21.845 | 36.292 | 5.355 | 1.00 | 29.01 | C |
| ATOM | 225 | CE1 | TYR | A | 222 | 24.417 | 37.194 | 4.802 | 1.00 | 30.48 | C |
| ATOM | 226 | CE2 | TYR | A | 222 | 22.034 | 37.540 | 4.754 | 1.00 | 31.64 | C |
| ATOM | 227 | CZ | TYR | A | 222 | 23.325 | 37.978 | 4.486 | 1.00 | 30.99 | C |
| ATOM | 228 | OH | TYR | A | 222 | 23.521 | 39.203 | 3.914 | 1.00 | 35.79 | O |
| ATOM | 229 | N | GLU | A | 223 | 25.060 | 33.634 | 8.479 | 1.00 | 27.59 | N |
| ATOM | 230 | CA | GLU | A | 223 | 26.459 | 33.791 | 8.865 | 1.00 | 28.49 | C |
| ATOM | 231 | C | GLU | A | 223 | 27.244 | 32.834 | 7.992 | 1.00 | 26.57 | C |
| ATOM | 232 | O | GLU | A | 223 | 26.787 | 31.724 | 7.732 | 1.00 | 25.86 | O |
| ATOM | 233 | CB | GLU | A | 223 | 26.668 | 33.437 | 10.345 | 1.00 | 28.97 | C |
| ATOM | 234 | CG | GLU | A | 223 | 25.978 | 34.403 | 11.303 | 1.00 | 32.90 | C |
| ATOM | 235 | CD | GLU | A | 223 | 26.366 | 34.187 | 12.762 | 1.00 | 34.83 | C |
| ATOM | 236 | OE1 | GLU | A | 223 | 26.061 | 33.115 | 13.319 | 1.00 | 35.25 | O |
| ATOM | 237 | OE2 | GLU | A | 223 | 26.984 | 35.100 | 13.349 | 1.00 | 37.75 | O |
| ATOM | 238 | N | VAL | A | 224 | 28.401 | 33.287 | 7.520 | 1.00 | 27.04 | N |
| ATOM | 239 | CA | VAL | A | 224 | 29.263 | 32.484 | 6.663 | 1.00 | 29.82 | C |
| ATOM | 240 | C | VAL | A | 224 | 30.605 | 32.314 | 7.348 | 1.00 | 32.25 | C |
| ATOM | 241 | O | VAL | A | 224 | 31.156 | 33.264 | 7.903 | 1.00 | 32.91 | O |
| ATOM | 242 | CB | VAL | A | 224 | 29.523 | 33.160 | 5.293 | 1.00 | 30.13 | C |
| ATOM | 243 | CG1 | VAL | A | 224 | 30.292 | 32.213 | 4.386 | 1.00 | 29.25 | C |
| ATOM | 244 | CG2 | VAL | A | 224 | 28.223 | 33.564 | 4.652 | 1.00 | 28.25 | C |
| ATOM | 245 | N | GLU | A | 225 | 31.127 | 31.095 | 7.313 | 1.00 | 36.25 | N |
| ATOM | 246 | CA | GLU | A | 225 | 32.409 | 30.800 | 7.926 | 1.00 | 38.31 | C |
| ATOM | 247 | C | GLU | A | 225 | 33.221 | 29.871 | 7.035 | 1.00 | 39.19 | C |
| ATOM | 248 | O | GLU | A | 225 | 32.743 | 28.817 | 6.613 | 1.00 | 37.16 | O |
| ATOM | 249 | CB | GLU | A | 225 | 32.179 | 30.184 | 9.306 | 1.00 | 38.76 | C |
| ATOM | 250 | CG | GLU | A | 225 | 31.591 | 31.192 | 10.291 | 1.00 | 43.19 | C |
| ATOM | 251 | CD | GLU | A | 225 | 30.900 | 30.557 | 11.490 | 1.00 | 44.45 | C |
| ATOM | 252 | OE1 | GLU | A | 225 | 30.377 | 31.320 | 12.336 | 1.00 | 45.47 | O |
| ATOM | 253 | OE2 | GLU | A | 225 | 30.874 | 29.310 | 11.588 | 1.00 | 45.46 | O |
| ATOM | 254 | N | ARG | A | 226 | 34.444 | 30.290 | 6.728 | 1.00 | 41.84 | N |
| ATOM | 255 | CA | ARG | A | 226 | 35.344 | 29.500 | 5.901 | 1.00 | 45.57 | C |
| ATOM | 256 | C | ARG | A | 226 | 35.748 | 28.257 | 6.684 | 1.00 | 46.77 | C |
| ATOM | 257 | O | ARG | A | 226 | 35.905 | 28.306 | 7.900 | 1.00 | 45.85 | O |
| ATOM | 258 | CB | ARG | A | 226 | 36.573 | 30.326 | 5.531 | 1.00 | 47.66 | C |
| ATOM | 259 | CG | ARG | A | 226 | 37.566 | 29.615 | 4.632 | 1.00 | 50.35 | C |
| ATOM | 260 | CD | ARG | A | 226 | 38.376 | 30.635 | 3.840 | 1.00 | 52.84 | C |
| ATOM | 261 | NE | ARG | A | 226 | 39.574 | 30.055 | 3.253 | 1.00 | 56.05 | N |
| ATOM | 262 | CZ | ARG | A | 226 | 40.642 | 29.684 | 3.953 | 1.00 | 58.25 | C |
| ATOM | 263 | NH1 | ARG | A | 226 | 40.663 | 29.835 | 5.268 | 1.00 | 58.31 | N |
| ATOM | 264 | NH2 | ARG | A | 226 | 41.696 | 29.160 | 3.338 | 1.00 | 61.87 | N |
| ATOM | 265 | N | MET | A | 227 | 35.913 | 27.148 | 5.970 | 1.00 | 50.54 | N |
| ATOM | 266 | CA | MET | A | 227 | 36.255 | 25.854 | 6.562 | 1.00 | 53.89 | C |
| ATOM | 267 | C | MET | A | 227 | 37.668 | 25.703 | 7.112 | 1.00 | 56.17 | C |
| ATOM | 268 | O | MET | A | 227 | 37.878 | 25.002 | 8.100 | 1.00 | 57.75 | O |
| ATOM | 269 | CB | MET | A | 227 | 36.029 | 24.749 | 5.529 | 1.00 | 53.39 | C |
| ATOM | 270 | CG | MET | A | 227 | 35.556 | 23.426 | 6.107 | 1.00 | 52.63 | C |
| ATOM | 271 | SD | MET | A | 227 | 33.807 | 23.453 | 6.512 | 1.00 | 50.86 | S |
| ATOM | 272 | CE | MET | A | 227 | 33.111 | 22.714 | 5.070 | 1.00 | 49.96 | C |
| ATOM | 273 | N | HIS | A | 228 | 38.634 | 26.345 | 6.466 | 1.00 | 58.17 | N |
| ATOM | 274 | CA | HIS | A | 228 | 40.034 | 26.242 | 6.873 | 1.00 | 60.05 | C |
| ATOM | 275 | C | HIS | A | 228 | 40.511 | 24.816 | 6.588 | 1.00 | 60.42 | C |
| ATOM | 276 | O | HIS | A | 228 | 40.118 | 23.862 | 7.263 | 1.00 | 59.78 | O |
| ATOM | 277 | CB | HIS | A | 228 | 40.208 | 26.567 | 8.357 | 1.00 | 60.65 | C |
| ATOM | 278 | CG | HIS | A | 228 | 41.637 | 26.739 | 8.766 | 1.00 | 62.18 | C |
| ATOM | 279 | CD2 | HIS | A | 228 | 42.288 | 27.794 | 9.311 | 1.00 | 62.26 | C |
| ATOM | 280 | ND1 | HIS | A | 228 | 42.581 | 25.747 | 8.605 | 1.00 | 62.16 | N |
| ATOM | 281 | CE1 | HIS | A | 228 | 43.752 | 26.185 | 9.033 | 1.00 | 62.59 | C |
| ATOM | 282 | NE2 | HIS | A | 228 | 43.602 | 27.423 | 9.466 | 1.00 | 62.46 | N |
| ATOM | 283 | N | ASN | A | 229 | 41.373 | 24.696 | 5.585 | 1.00 | 60.91 | N |
| ATOM | 284 | CA | ASN | A | 229 | 41.910 | 23.421 | 5.124 | 1.00 | 62.13 | C |
| ATOM | 285 | C | ASN | A | 229 | 42.763 | 22.628 | 6.107 | 1.00 | 63.61 | C |
| ATOM | 286 | O | ASN | A | 229 | 42.680 | 21.403 | 6.150 | 1.00 | 63.55 | O |
| ATOM | 287 | CB | ASN | A | 229 | 42.702 | 23.668 | 3.840 | 1.00 | 60.59 | C |
| ATOM | 288 | CG | ASN | A | 229 | 41.921 | 24.493 | 2.837 | 1.00 | 59.28 | C |
| ATOM | 289 | ND2 | ASN | A | 229 | 40.599 | 24.491 | 2.978 | 1.00 | 57.96 | N |
| ATOM | 290 | OD1 | ASN | A | 229 | 42.494 | 25.130 | 1.951 | 1.00 | 59.51 | O |
| ATOM | 291 | N | ASP | A | 230 | 43.583 | 23.320 | 6.889 | 1.00 | 66.32 | N |
| ATOM | 292 | CA | ASP | A | 230 | 44.460 | 22.656 | 7.852 | 1.00 | 68.70 | C |
| ATOM | 293 | C | ASP | A | 230 | 43.784 | 22.228 | 9.161 | 1.00 | 69.70 | C |
| ATOM | 294 | O | ASP | A | 230 | 43.521 | 21.044 | 9.374 | 1.00 | 69.69 | O |
| ATOM | 295 | CB | ASP | A | 230 | 45.669 | 23.552 | 8.169 | 1.00 | 70.13 | C |
| ATOM | 296 | CG | ASP | A | 230 | 46.660 | 23.642 | 7.008 | 1.00 | 71.10 | C |
| ATOM | 297 | OD1 | ASP | A | 230 | 46.248 | 24.011 | 5.886 | 1.00 | 72.83 | O |
| ATOM | 298 | OD2 | ASP | A | 230 | 47.857 | 23.345 | 7.218 | 1.00 | 71.05 | O |
| ATOM | 299 | N | THR | A | 231 | 43.499 | 23.189 | 10.033 | 1.00 | 70.85 | N |
| ATOM | 300 | CA | THR | A | 231 | 42.895 | 22.893 | 11.331 | 1.00 | 71.32 | C |
| ATOM | 301 | C | THR | A | 231 | 41.402 | 22.573 | 11.349 | 1.00 | 71.39 | C |
| ATOM | 302 | O | THR | A | 231 | 40.771 | 22.367 | 10.314 | 1.00 | 71.12 | O |
| ATOM | 303 | CB | THR | A | 231 | 43.140 | 24.045 | 12.306 | 1.00 | 71.30 | C |
| ATOM | 304 | CG2 | THR | A | 231 | 44.628 | 24.293 | 12.465 | 1.00 | 71.54 | C |
| ATOM | 305 | OG1 | THR | A | 231 | 42.515 | 25.230 | 11.804 | 1.00 | 72.28 | O |
| ATOM | 306 | N | TRP | A | 232 | 40.849 | 22.545 | 12.556 | 1.00 | 71.33 | N |
| ATOM | 307 | CA | TRP | A | 232 | 39.440 | 22.237 | 12.775 | 1.00 | 72.09 | C |
| ATOM | 308 | C | TRP | A | 232 | 38.591 | 23.486 | 12.926 | 1.00 | 70.71 | C |
| ATOM | 309 | O | TRP | A | 232 | 37.391 | 23.387 | 13.152 | 1.00 | 70.50 | O |
| ATOM | 310 | CB | TRP | A | 232 | 39.271 | 21.441 | 14.068 | 1.00 | 75.07 | C |
| ATOM | 311 | CG | TRP | A | 232 | 40.023 | 20.159 | 14.174 | 1.00 | 78.63 | C |
| ATOM | 312 | CD1 | TRP | A | 232 | 41.273 | 19.879 | 13.692 | 1.00 | 79.38 | C |
| ATOM | 313 | CD2 | TRP | A | 232 | 39.602 | 18.998 | 14.892 | 1.00 | 80.08 | C |
| ATOM | 314 | CE2 | TRP | A | 232 | 40.646 | 18.051 | 14.811 | 1.00 | 80.83 | C |
| ATOM | 315 | CE3 | TRP | A | 232 | 38.440 | 18.668 | 15.604 | 1.00 | 81.30 | C |
| ATOM | 316 | NE1 | TRP | A | 232 | 41.653 | 18.612 | 14.071 | 1.00 | 79.96 | N |
| ATOM | 317 | CZ2 | TRP | A | 232 | 40.561 | 16.791 | 15.414 | 1.00 | 81.85 | C |
| ATOM | 318 | CZ3 | TRP | A | 232 | 38.355 | 17.417 | 16.203 | 1.00 | 82.02 | C |
| ATOM | 319 | CH2 | TRP | A | 232 | 39.411 | 16.493 | 16.104 | 1.00 | 82.42 | C |
| ATOM | 320 | N | VAL | A | 233 | 39.193 | 24.659 | 12.817 | 1.00 | 69.27 | N |
| ATOM | 321 | CA | VAL | A | 233 | 38.422 | 25.873 | 13.022 | 1.00 | 67.77 | C |
| ATOM | 322 | C | VAL | A | 233 | 37.715 | 26.474 | 11.825 | 1.00 | 66.44 | C |
| ATOM | 323 | O | VAL | A | 233 | 38.009 | 26.155 | 10.677 | 1.00 | 66.91 | O |
| ATOM | 324 | CB | VAL | A | 233 | 39.292 | 26.964 | 13.654 | 1.00 | 68.11 | C |
| ATOM | 325 | CG1 | VAL | A | 233 | 39.771 | 26.503 | 15.012 | 1.00 | 68.39 | C |
| ATOM | 326 | CG2 | VAL | A | 233 | 40.468 | 27.276 | 12.753 | 1.00 | 68.40 | C |
| ATOM | 327 | N | LEU | A | 234 | 36.760 | 27.347 | 12.127 | 1.00 | 64.47 | N |
| ATOM | 328 | CA | LEU | A | 234 | 35.983 | 28.047 | 11.117 | 1.00 | 62.87 | C |
| ATOM | 329 | C | LEU | A | 234 | 36.403 | 29.512 | 11.187 | 1.00 | 63.00 | C |
| ATOM | 330 | O | LEU | A | 234 | 36.796 | 29.991 | 12.251 | 1.00 | 62.19 | O |
| ATOM | 331 | CB | LEU | A | 234 | 34.492 | 27.916 | 11.424 | 1.00 | 61.01 | C |
| ATOM | 332 | CG | LEU | A | 234 | 33.920 | 26.496 | 11.461 | 1.00 | 60.06 | C |
| ATOM | 333 | CD1 | LEU | A | 234 | 32.553 | 26.523 | 12.121 | 1.00 | 59.17 | C |
| ATOM | 334 | CD2 | LEU | A | 234 | 33.840 | 25.927 | 10.052 | 1.00 | 58.70 | C |
| ATOM | 335 | N | LEU | A | 235 | 36.334 | 30.219 | 10.061 | 1.00 | 63.41 | N |
| ATOM | 336 | CA | LEU | A | 235 | 36.719 | 31.632 | 10.024 | 1.00 | 63.22 | C |
| ATOM | 337 | C | LEU | A | 235 | 35.561 | 32.562 | 9.673 | 1.00 | 62.75 | C |
| ATOM | 338 | O | LEU | A | 235 | 34.844 | 32.343 | 8.695 | 1.00 | 61.83 | O |
| ATOM | 339 | CB | LEU | A | 235 | 37.861 | 31.856 | 9.021 | 1.00 | 63.54 | C |
| ATOM | 340 | CG | LEU | A | 235 | 38.289 | 33.315 | 8.776 | 1.00 | 63.51 | C |
| ATOM | 341 | CD1 | LEU | A | 235 | 38.828 | 33.928 | 10.057 | 1.00 | 63.54 | C |
| ATOM | 342 | CD2 | LEU | A | 235 | 39.347 | 33.368 | 7.683 | 1.00 | 63.54 | C |
| ATOM | 343 | N | ASN | A | 236 | 35.396 | 33.606 | 10.481 | 1.00 | 62.45 | N |
| ATOM | 344 | CA | ASN | A | 236 | 34.345 | 34.597 | 10.276 | 1.00 | 62.02 | C |
| ATOM | 345 | C | ASN | A | 236 | 34.516 | 35.191 | 8.888 | 1.00 | 61.48 | C |
| ATOM | 346 | O | ASN | A | 236 | 35.589 | 35.686 | 8.548 | 1.00 | 62.41 | O |
| ATOM | 347 | CB | ASN | A | 236 | 34.461 | 35.711 | 11.318 | 1.00 | 63.38 | C |
| ATOM | 348 | CG | ASN | A | 236 | 34.460 | 35.183 | 12.737 | 1.00 | 64.80 | C |
| ATOM | 349 | ND2 | ASN | A | 236 | 34.063 | 33.926 | 12.902 | 1.00 | 65.97 | N |
| ATOM | 350 | OD1 | ASN | A | 236 | 34.808 | 35.898 | 13.678 | 1.00 | 65.10 | O |
| ATOM | 351 | N | GLN | A | 237 | 33.458 | 35.142 | 8.087 | 1.00 | 59.84 | N |
| ATOM | 352 | CA | GLN | A | 237 | 33.503 | 35.673 | 6.732 | 1.00 | 58.07 | C |
| ATOM | 353 | C | GLN | A | 237 | 32.587 | 36.889 | 6.581 | 1.00 | 55.95 | C |
| ATOM | 354 | O | GLN | A | 237 | 33.030 | 37.961 | 6.166 | 1.00 | 55.15 | O |
| ATOM | 355 | CB | GLN | A | 237 | 33.107 | 34.583 | 5.735 | 1.00 | 60.15 | C |
| ATOM | 356 | CG | GLN | A | 237 | 34.048 | 33.385 | 5.717 | 1.00 | 62.58 | C |
| ATOM | 357 | CD | GLN | A | 237 | 35.482 | 33.768 | 5.380 | 1.00 | 63.94 | C |
| ATOM | 358 | NE2 | GLN | A | 237 | 36.423 | 33.346 | 6.223 | 1.00 | 64.93 | N |
| ATOM | 359 | OE1 | GLN | A | 237 | 35.741 | 34.428 | 4.373 | 1.00 | 63.69 | O |
| ATOM | 360 | N | ARG | A | 238 | 31.312 | 36.716 | 6.912 | 1.00 | 52.32 | N |
| ATOM | 361 | CA | ARG | A | 238 | 30.344 | 37.803 | 6.835 | 1.00 | 48.41 | C |
| ATOM | 362 | C | ARG | A | 238 | 29.000 | 37.407 | 7.436 | 1.00 | 44.26 | C |
| ATOM | 363 | O | ARG | A | 238 | 28.771 | 36.246 | 7.777 | 1.00 | 40.97 | O |
| ATOM | 364 | CB | ARG | A | 238 | 30.154 | 38.267 | 5.386 | 1.00 | 51.23 | C |
| ATOM | 365 | CG | ARG | A | 238 | 29.726 | 37.188 | 4.416 | 1.00 | 54.87 | C |
| ATOM | 366 | CD | ARG | A | 238 | 30.521 | 37.305 | 3.124 | 1.00 | 57.96 | C |
| ATOM | 367 | NE | ARG | A | 238 | 31.957 | 37.186 | 3.383 | 1.00 | 60.60 | N |
| ATOM | 368 | CZ | ARG | A | 238 | 32.900 | 37.258 | 2.449 | 1.00 | 61.70 | C |
| ATOM | 369 | NH1 | ARG | A | 238 | 32.569 | 37.451 | 1.179 | 1.00 | 62.68 | N |
| ATOM | 370 | NH2 | ARG | A | 238 | 34.177 | 37.130 | 2.785 | 1.00 | 61.92 | N |
| ATOM | 371 | N | ARG | A | 239 | 28.114 | 38.383 | 7.574 | 1.00 | 40.37 | N |
| ATOM | 372 | CA | ARG | A | 239 | 26.816 | 38.124 | 8.148 | 1.00 | 38.77 | C |
| ATOM | 373 | C | ARG | A | 239 | 25.816 | 39.173 | 7.708 | 1.00 | 35.72 | C |
| ATOM | 374 | O | ARG | A | 239 | 26.195 | 40.225 | 7.191 | 1.00 | 34.30 | O |
| ATOM | 375 | CB | ARG | A | 239 | 26.906 | 38.101 | 9.683 | 1.00 | 41.55 | C |
| ATOM | 376 | CG | ARG | A | 239 | 27.437 | 39.386 | 10.311 | 1.00 | 45.95 | C |
| ATOM | 377 | CD | ARG | A | 239 | 27.173 | 39.434 | 11.819 | 1.00 | 50.14 | C |
| ATOM | 378 | NE | ARG | A | 239 | 27.765 | 38.300 | 12.530 | 1.00 | 53.72 | N |
| ATOM | 379 | CZ | ARG | A | 239 | 29.069 | 38.142 | 12.740 | 1.00 | 56.30 | C |
| ATOM | 380 | NH1 | ARG | A | 239 | 29.931 | 39.048 | 12.298 | 1.00 | 55.86 | N |
| ATOM | 381 | NH2 | ARG | A | 239 | 29.512 | 37.075 | 13.390 | 1.00 | 57.31 | N |
| ATOM | 382 | N | GLY | A | 240 | 24.537 | 38.875 | 7.917 | 1.00 | 31.85 | N |
| ATOM | 383 | CA | GLY | A | 240 | 23.486 | 39.796 | 7.537 | 1.00 | 28.04 | C |
| ATOM | 384 | C | GLY | A | 240 | 22.120 | 39.161 | 7.702 | 1.00 | 25.84 | C |
| ATOM | 385 | O | GLY | A | 240 | 22.014 | 38.023 | 8.163 | 1.00 | 24.77 | O |
| ATOM | 386 | N | PHE | A | 241 | 21.073 | 39.895 | 7.339 | 1.00 | 22.89 | N |
| ATOM | 387 | CA | PHE | A | 241 | 19.720 | 39.375 | 7.440 | 1.00 | 23.28 | C |
| ATOM | 388 | C | PHE | A | 241 | 18.848 | 39.998 | 6.358 | 1.00 | 23.87 | C |
| ATOM | 389 | O | PHE | A | 241 | 19.233 | 40.984 | 5.733 | 1.00 | 22.98 | O |
| ATOM | 390 | CB | PHE | A | 241 | 19.128 | 39.662 | 8.826 | 1.00 | 21.58 | C |
| ATOM | 391 | CG | PHE | A | 241 | 18.573 | 41.040 | 8.974 | 1.00 | 23.25 | C |
| ATOM | 392 | CD1 | PHE | A | 241 | 17.209 | 41.272 | 8.840 | 1.00 | 26.30 | C |
| ATOM | 393 | CD2 | PHE | A | 241 | 19.410 | 42.110 | 9.230 | 1.00 | 24.10 | C |
| ATOM | 394 | CE1 | PHE | A | 241 | 16.685 | 42.561 | 8.960 | 1.00 | 26.80 | C |
| ATOM | 395 | CE2 | PHE | A | 241 | 18.900 | 43.400 | 9.354 | 1.00 | 29.27 | C |
| ATOM | 396 | CZ | PHE | A | 241 | 17.527 | 43.625 | 9.218 | 1.00 | 27.60 | C |
| ATOM | 397 | N | LEU | A | 242 | 17.666 | 39.427 | 6.151 | 1.00 | 24.90 | N |
| ATOM | 398 | CA | LEU | A | 242 | 16.748 | 39.925 | 5.138 | 1.00 | 25.37 | C |
| ATOM | 399 | C | LEU | A | 242 | 15.365 | 39.307 | 5.330 | 1.00 | 25.69 | C |
| ATOM | 400 | O | LEU | A | 242 | 15.189 | 38.428 | 6.164 | 1.00 | 24.82 | O |
| ATOM | 401 | CB | LEU | A | 242 | 17.294 | 39.583 | 3.746 | 1.00 | 25.14 | C |
| ATOM | 402 | CG | LEU | A | 242 | 17.509 | 38.099 | 3.414 | 1.00 | 23.89 | C |
| ATOM | 403 | CD1 | LEU | A | 242 | 16.209 | 37.472 | 2.927 | 1.00 | 21.96 | C |
| ATOM | 404 | CD2 | LEU | A | 242 | 18.581 | 37.992 | 2.329 | 1.00 | 25.06 | C |
| ATOM | 405 | N | CYS | A | 243 | 14.380 | 39.783 | 4.578 | 1.00 | 26.81 | N |
| ATOM | 406 | CA | CYS | A | 243 | 13.040 | 39.220 | 4.686 | 1.00 | 29.06 | C |
| ATOM | 407 | C | CYS | A | 243 | 12.488 | 39.100 | 3.280 | 1.00 | 28.71 | C |
| ATOM | 408 | O | CYS | A | 243 | 13.091 | 39.599 | 2.334 | 1.00 | 27.84 | O |
| ATOM | 409 | CB | CYS | A | 243 | 12.137 | 40.102 | 5.558 | 1.00 | 32.37 | C |
| ATOM | 410 | SG | CYS | A | 243 | 11.668 | 41.674 | 4.828 | 1.00 | 39.37 | S |
| ATOM | 411 | N | ASN | A | 244 | 11.369 | 38.413 | 3.132 | 1.00 | 28.76 | N |
| ATOM | 412 | CA | ASN | A | 244 | 10.774 | 38.242 | 1.818 | 1.00 | 32.53 | C |
| ATOM | 413 | C | ASN | A | 244 | 10.430 | 39.599 | 1.204 | 1.00 | 35.61 | C |
| ATOM | 414 | O | ASN | A | 244 | 10.418 | 40.615 | 1.891 | 1.00 | 35.33 | O |
| ATOM | 415 | CB | ASN | A | 244 | 9.500 | 37.439 | 1.942 | 1.00 | 30.71 | C |
| ATOM | 416 | CG | ASN | A | 244 | 8.456 | 38.174 | 2.731 | 1.00 | 31.47 | C |
| ATOM | 417 | ND2 | ASN | A | 244 | 7.429 | 38.640 | 2.037 | 1.00 | 28.56 | N |
| ATOM | 418 | OD1 | ASN | A | 244 | 8.580 | 38.353 | 3.951 | 1.00 | 28.63 | O |
| ATOM | 419 | N | GLN | A | 245 | 10.145 | 39.599 | −0.096 | 1.00 | 40.74 | N |
| ATOM | 420 | CA | GLN | A | 245 | 9.786 | 40.819 | −0.817 | 1.00 | 44.22 | C |
| ATOM | 421 | C | GLN | A | 245 | 8.364 | 40.712 | −1.352 | 1.00 | 44.57 | C |
| ATOM | 422 | O | GLN | A | 245 | 8.147 | 40.227 | −2.463 | 1.00 | 44.96 | O |
| ATOM | 423 | CB | GLN | A | 245 | 10.749 | 41.054 | −1.990 | 1.00 | 45.76 | C |
| ATOM | 424 | CG | GLN | A | 245 | 12.191 | 41.308 | −1.585 | 1.00 | 48.31 | C |
| ATOM | 425 | CD | GLN | A | 245 | 12.351 | 42.558 | −0.732 | 1.00 | 51.02 | C |
| ATOM | 426 | NE2 | GLN | A | 245 | 12.830 | 42.380 | 0.499 | 1.00 | 51.12 | N |
| ATOM | 427 | OE1 | GLN | A | 245 | 12.046 | 43.670 | −1.173 | 1.00 | 51.70 | O |
| ATOM | 428 | N | ALA | A | 246 | 7.404 | 41.170 | −0.554 | 1.00 | 45.02 | N |
| ATOM | 429 | CA | ALA | A | 246 | 5.993 | 41.140 | −0.927 | 1.00 | 46.95 | C |
| ATOM | 430 | C | ALA | A | 246 | 5.764 | 41.516 | −2.388 | 1.00 | 48.20 | C |
| ATOM | 431 | O | ALA | A | 246 | 6.507 | 42.312 | −2.962 | 1.00 | 46.28 | O |
| ATOM | 432 | CB | ALA | A | 246 | 5.205 | 42.075 | −0.029 | 1.00 | 47.31 | C |
| ATOM | 433 | N | PRO | A | 247 | 4.731 | 40.931 | −3.016 | 1.00 | 50.66 | N |
| ATOM | 434 | CA | PRO | A | 247 | 4.442 | 41.240 | −4.418 | 1.00 | 52.70 | C |
| ATOM | 435 | C | PRO | A | 247 | 4.292 | 42.749 | −4.606 | 1.00 | 54.26 | C |
| ATOM | 436 | O | PRO | A | 247 | 3.644 | 43.427 | −3.808 | 1.00 | 53.36 | O |
| ATOM | 437 | CB | PRO | A | 247 | 3.147 | 40.479 | −4.674 | 1.00 | 52.33 | C |
| ATOM | 438 | CG | PRO | A | 247 | 3.308 | 39.273 | −3.794 | 1.00 | 51.89 | C |
| ATOM | 439 | CD | PRO | A | 247 | 3.823 | 39.885 | −2.514 | 1.00 | 50.74 | C |
| ATOM | 440 | N | HIS | A | 248 | 4.915 | 43.267 | −5.656 | 1.00 | 56.23 | N |
| ATOM | 441 | CA | HIS | A | 248 | 4.864 | 44.689 | −5.941 | 1.00 | 58.62 | C |
| ATOM | 442 | C | HIS | A | 248 | 4.866 | 44.900 | −7.450 | 1.00 | 60.49 | C |
| ATOM | 443 | O | HIS | A | 248 | 5.593 | 44.223 | −8.183 | 1.00 | 59.80 | O |
| ATOM | 444 | CB | HIS | A | 248 | 6.076 | 45.385 | −5.325 | 1.00 | 59.35 | C |
| ATOM | 445 | CG | HIS | A | 248 | 5.891 | 46.855 | −5.118 | 1.00 | 61.26 | C |
| ATOM | 446 | CD2 | HIS | A | 248 | 6.409 | 47.922 | −5.772 | 1.00 | 61.78 | C |
| ATOM | 447 | ND1 | HIS | A | 248 | 5.086 | 47.369 | −4.124 | 1.00 | 61.25 | N |
| ATOM | 448 | CE1 | HIS | A | 248 | 5.119 | 48.688 | −4.172 | 1.00 | 62.37 | C |
| ATOM | 449 | NE2 | HIS | A | 248 | 5.915 | 49.050 | −5.163 | 1.00 | 62.42 | N |
| ATOM | 450 | N | LYS | A | 249 | 4.051 | 45.846 | −7.906 | 1.00 | 62.41 | N |
| ATOM | 451 | CA | LYS | A | 249 | 3.949 | 46.156 | −9.325 | 1.00 | 64.67 | C |
| ATOM | 452 | C | LYS | A | 249 | 5.227 | 46.810 | −9.849 | 1.00 | 65.45 | C |
| ATOM | 453 | O | LYS | A | 249 | 5.529 | 46.728 | −11.038 | 1.00 | 65.16 | O |
| ATOM | 454 | CB | LYS | A | 249 | 2.745 | 47.072 | −9.577 | 1.00 | 65.55 | C |
| ATOM | 455 | CG | LYS | A | 249 | 1.414 | 46.464 | −9.150 | 1.00 | 66.11 | C |
| ATOM | 456 | CD | LYS | A | 249 | 0.232 | 47.373 | −9.456 | 1.00 | 66.49 | C |
| ATOM | 457 | CE | LYS | A | 249 | −1.069 | 46.756 | −8.956 | 1.00 | 66.21 | C |
| ATOM | 458 | NZ | LYS | A | 249 | −1.310 | 45.420 | −9.570 | 1.00 | 65.64 | N |
| ATOM | 459 | N | HIS | A | 250 | 5.977 | 47.452 | −8.956 | 1.00 | 67.03 | N |
| ATOM | 460 | CA | HIS | A | 250 | 7.222 | 48.116 | −9.334 | 1.00 | 68.32 | C |
| ATOM | 461 | C | HIS | A | 250 | 8.434 | 47.241 | −9.018 | 1.00 | 68.34 | C |
| ATOM | 462 | O | HIS | A | 250 | 9.576 | 47.691 | −9.113 | 1.00 | 67.90 | O |
| ATOM | 463 | CB | HIS | A | 250 | 7.339 | 49.465 | −8.614 | 1.00 | 68.65 | C |
| ATOM | 464 | CG | HIS | A | 250 | 6.308 | 50.467 | −9.039 | 1.00 | 70.43 | C |
| ATOM | 465 | CD2 | HIS | A | 250 | 4.989 | 50.566 | −8.745 | 1.00 | 70.92 | C |
| ATOM | 466 | ND1 | HIS | A | 250 | 6.586 | 51.507 | −9.901 | 1.00 | 71.36 | N |
| ATOM | 467 | CE1 | HIS | A | 250 | 5.483 | 52.202 | −10.119 | 1.00 | 71.79 | C |
| ATOM | 468 | NE2 | HIS | A | 250 | 4.500 | 51.652 | −9.430 | 1.00 | 71.57 | N |
| ATOM | 469 | N | GLY | A | 251 | 8.172 | 45.986 | −8.651 | 1.00 | 69.38 | N |
| ATOM | 470 | CA | GLY | A | 251 | 9.237 | 45.048 | −8.328 | 1.00 | 70.10 | C |
| ATOM | 471 | C | GLY | A | 251 | 8.885 | 43.642 | −8.777 | 1.00 | 70.84 | C |
| ATOM | 472 | O | GLY | A | 251 | 8.471 | 43.440 | −9.918 | 1.00 | 70.52 | O |
| ATOM | 473 | N | PHE | A | 252 | 9.043 | 42.666 | −7.889 | 1.00 | 71.70 | N |
| ATOM | 474 | CA | PHE | A | 252 | 8.726 | 41.280 | −8.226 | 1.00 | 72.59 | C |
| ATOM | 475 | C | PHE | A | 252 | 7.211 | 41.062 | −8.214 | 1.00 | 72.46 | C |
| ATOM | 476 | O | PHE | A | 252 | 6.607 | 40.815 | −7.166 | 1.00 | 71.73 | O |
| ATOM | 477 | CB | PHE | A | 252 | 9.396 | 40.310 | −7.241 | 1.00 | 74.22 | C |
| ATOM | 478 | CG | PHE | A | 252 | 10.897 | 40.456 | −7.156 | 1.00 | 75.87 | C |
| ATOM | 479 | CD1 | PHE | A | 252 | 11.469 | 41.441 | −6.359 | 1.00 | 76.35 | C |
| ATOM | 480 | CD2 | PHE | A | 252 | 11.737 | 39.609 | −7.876 | 1.00 | 76.69 | C |
| ATOM | 481 | CE1 | PHE | A | 252 | 12.855 | 41.579 | −6.273 | 1.00 | 77.05 | C |
| ATOM | 482 | CE2 | PHE | A | 252 | 13.128 | 39.738 | −7.798 | 1.00 | 76.99 | C |
| ATOM | 483 | CZ | PHE | A | 252 | 13.686 | 40.727 | −6.996 | 1.00 | 76.88 | C |
| ATOM | 484 | N | LEU | A | 253 | 6.608 | 41.161 | −9.392 | 1.00 | 72.48 | N |
| ATOM | 485 | CA | LEU | A | 253 | 5.169 | 40.987 | −9.556 | 1.00 | 72.39 | C |
| ATOM | 486 | C | LEU | A | 253 | 4.599 | 39.821 | −8.757 | 1.00 | 71.11 | C |
| ATOM | 487 | O | LEU | A | 253 | 3.692 | 40.000 | −7.942 | 1.00 | 71.11 | O |
| ATOM | 488 | CB | LEU | A | 253 | 4.846 | 40.790 | −11.041 | 1.00 | 74.32 | C |
| ATOM | 489 | CG | LEU | A | 253 | 3.411 | 40.442 | −11.441 | 1.00 | 75.31 | C |
| ATOM | 490 | CD1 | LEU | A | 253 | 2.475 | 41.580 | −11.081 | 1.00 | 76.41 | C |
| ATOM | 491 | CD2 | LEU | A | 253 | 3.366 | 40.170 | −12.932 | 1.00 | 76.23 | C |
| ATOM | 492 | N | GLU | A | 254 | 5.140 | 38.629 | −8.994 | 1.00 | 69.56 | N |
| ATOM | 493 | CA | GLU | A | 254 | 4.667 | 37.419 | −8.327 | 1.00 | 67.61 | C |
| ATOM | 494 | C | GLU | A | 254 | 5.310 | 37.157 | −6.958 | 1.00 | 65.21 | C |
| ATOM | 495 | O | GLU | A | 254 | 5.159 | 36.076 | −6.392 | 1.00 | 66.04 | O |
| ATOM | 496 | CB | GLU | A | 254 | 4.878 | 36.205 | −9.247 | 1.00 | 69.16 | C |
| ATOM | 497 | CG | GLU | A | 254 | 4.005 | 34.988 | −8.914 | 1.00 | 71.80 | C |
| ATOM | 498 | CD | GLU | A | 254 | 2.550 | 35.157 | −9.341 | 1.00 | 72.89 | C |
| ATOM | 499 | OE1 | GLU | A | 254 | 1.712 | 34.306 | −8.988 | 1.00 | 74.54 | O |
| ATOM | 500 | OE2 | GLU | A | 254 | 2.246 | 36.139 | −10.050 | 1.00 | 73.37 | O |
| ATOM | 501 | N | GLY | A | 255 | 6.031 | 38.140 | −6.431 | 1.00 | 61.33 | N |
| ATOM | 502 | CA | GLY | A | 255 | 6.643 | 37.979 | −5.125 | 1.00 | 55.33 | C |
| ATOM | 503 | C | GLY | A | 255 | 8.001 | 37.301 | −5.070 | 1.00 | 50.81 | C |
| ATOM | 504 | O | GLY | A | 255 | 8.323 | 36.432 | −5.886 | 1.00 | 51.84 | O |
| ATOM | 505 | N | ARG | A | 256 | 8.795 | 37.699 | −4.080 | 1.00 | 43.54 | N |
| ATOM | 506 | CA | ARG | A | 256 | 10.131 | 37.155 | −3.898 | 1.00 | 36.96 | C |
| ATOM | 507 | C | ARG | A | 256 | 10.285 | 36.672 | −2.459 | 1.00 | 33.56 | C |
| ATOM | 508 | O | ARG | A | 256 | 10.374 | 37.466 | −1.522 | 1.00 | 30.10 | O |
| ATOM | 509 | CB | ARG | A | 256 | 11.170 | 38.238 | −4.213 | 1.00 | 36.58 | C |
| ATOM | 510 | CG | ARG | A | 256 | 12.585 | 37.728 | −4.448 | 1.00 | 35.18 | C |
| ATOM | 511 | CD | ARG | A | 256 | 12.657 | 36.953 | −5.744 | 1.00 | 34.60 | C |
| ATOM | 512 | NE | ARG | A | 256 | 13.986 | 36.435 | −6.056 | 1.00 | 30.07 | N |
| ATOM | 513 | CZ | ARG | A | 256 | 14.221 | 35.651 | −7.098 | 1.00 | 27.41 | C |
| ATOM | 514 | NH1 | ARG | A | 256 | 13.203 | 35.331 | −7.886 | 1.00 | 26.27 | N |
| ATOM | 515 | NH2 | ARG | A | 256 | 15.445 | 35.177 | −7.346 | 1.00 | 23.45 | N |
| ATOM | 516 | N | HIS | A | 257 | 10.318 | 35.357 | −2.291 | 1.00 | 30.01 | N |
| ATOM | 517 | CA | HIS | A | 257 | 10.439 | 34.780 | −0.967 | 1.00 | 27.31 | C |
| ATOM | 518 | C | HIS | A | 257 | 11.837 | 34.972 | −0.394 | 1.00 | 25.30 | C |
| ATOM | 519 | O | HIS | A | 257 | 12.824 | 35.032 | −1.130 | 1.00 | 23.97 | O |
| ATOM | 520 | CB | HIS | A | 257 | 10.033 | 33.300 | −1.008 | 1.00 | 28.84 | C |
| ATOM | 521 | CG | HIS | A | 257 | 8.567 | 33.092 | −1.242 | 1.00 | 29.60 | C |
| ATOM | 522 | CD2 | HIS | A | 257 | 7.551 | 33.985 | −1.344 | 1.00 | 29.10 | C |
| ATOM | 523 | ND1 | HIS | A | 257 | 7.997 | 31.845 | −1.374 | 1.00 | 29.06 | N |
| ATOM | 524 | CE1 | HIS | A | 257 | 6.692 | 31.976 | −1.547 | 1.00 | 29.26 | C |
| ATOM | 525 | NE2 | HIS | A | 257 | 6.396 | 33.263 | −1.533 | 1.00 | 29.96 | N |
| ATOM | 526 | N | ALA | A | 258 | 11.900 | 35.094 | 0.929 | 1.00 | 21.10 | N |
| ATOM | 527 | CA | ALA | A | 258 | 13.140 | 35.319 | 1.639 | 1.00 | 19.97 | C |
| ATOM | 528 | C | ALA | A | 258 | 14.226 | 34.346 | 1.217 | 1.00 | 21.67 | C |
| ATOM | 529 | O | ALA | A | 258 | 15.389 | 34.719 | 1.034 | 1.00 | 20.65 | O |
| ATOM | 530 | CB | ALA | A | 258 | 12.899 | 35.197 | 3.129 | 1.00 | 19.45 | C |
| ATOM | 531 | N | GLU | A | 259 | 13.835 | 33.089 | 1.065 | 1.00 | 21.64 | N |
| ATOM | 532 | CA | GLU | A | 259 | 14.772 | 32.056 | 0.675 | 1.00 | 21.05 | C |
| ATOM | 533 | C | GLU | A | 259 | 15.364 | 32.333 | −0.700 | 1.00 | 21.70 | C |
| ATOM | 534 | O | GLU | A | 259 | 16.566 | 32.132 | −0.920 | 1.00 | 21.86 | O |
| ATOM | 535 | CB | GLU | A | 259 | 14.086 | 30.690 | 0.703 | 1.00 | 21.94 | C |
| ATOM | 536 | CG | GLU | A | 259 | 13.668 | 30.236 | 2.106 | 1.00 | 23.41 | C |
| ATOM | 537 | CD | GLU | A | 259 | 12.358 | 30.845 | 2.569 | 1.00 | 22.93 | C |
| ATOM | 538 | OE1 | GLU | A | 259 | 11.864 | 30.442 | 3.639 | 1.00 | 23.62 | O |
| ATOM | 539 | OE2 | GLU | A | 259 | 11.818 | 31.729 | 1.867 | 1.00 | 24.99 | O |
| ATOM | 540 | N | LEU | A | 260 | 14.531 | 32.800 | −1.624 | 1.00 | 19.90 | N |
| ATOM | 541 | CA | LEU | A | 260 | 15.020 | 33.110 | −2.957 | 1.00 | 21.88 | C |
| ATOM | 542 | C | LEU | A | 260 | 15.905 | 34.359 | −2.916 | 1.00 | 21.85 | C |
| ATOM | 543 | O | LEU | A | 260 | 16.891 | 34.453 | −3.660 | 1.00 | 21.02 | O |
| ATOM | 544 | CB | LEU | A | 260 | 13.851 | 33.316 | −3.929 | 1.00 | 21.55 | C |
| ATOM | 545 | CG | LEU | A | 260 | 13.003 | 32.079 | −4.253 | 1.00 | 22.51 | C |
| ATOM | 546 | CD1 | LEU | A | 260 | 11.951 | 32.434 | −5.315 | 1.00 | 23.25 | C |
| ATOM | 547 | CD2 | LEU | A | 260 | 13.888 | 30.962 | −4.761 | 1.00 | 19.81 | C |
| ATOM | 548 | N | CYS | A | 261 | 15.558 | 35.312 | −2.050 | 1.00 | 20.65 | N |
| ATOM | 549 | CA | CYS | A | 261 | 16.363 | 36.528 | −1.922 | 1.00 | 23.28 | C |
| ATOM | 550 | C | CYS | A | 261 | 17.726 | 36.164 | −1.331 | 1.00 | 23.65 | C |
| ATOM | 551 | O | CYS | A | 261 | 18.748 | 36.718 | −1.721 | 1.00 | 23.08 | O |
| ATOM | 552 | CB | CYS | A | 261 | 15.681 | 37.559 | −1.013 | 1.00 | 24.60 | C |
| ATOM | 553 | SG | CYS | A | 261 | 14.135 | 38.256 | −1.644 | 1.00 | 27.70 | S |
| ATOM | 554 | N | PHE | A | 262 | 17.734 | 35.235 | −0.383 | 1.00 | 22.49 | N |
| ATOM | 555 | CA | PHE | A | 262 | 18.986 | 34.812 | 0.220 | 1.00 | 21.43 | C |
| ATOM | 556 | C | PHE | A | 262 | 19.927 | 34.276 | −0.864 | 1.00 | 20.50 | C |
| ATOM | 557 | O | PHE | A | 262 | 21.075 | 34.693 | −0.956 | 1.00 | 19.62 | O |
| ATOM | 558 | CB | PHE | A | 262 | 18.710 | 33.738 | 1.271 | 1.00 | 21.46 | C |
| ATOM | 559 | CG | PHE | A | 262 | 19.938 | 33.055 | 1.783 | 1.00 | 21.54 | C |
| ATOM | 560 | CD1 | PHE | A | 262 | 20.991 | 33.787 | 2.333 | 1.00 | 19.94 | C |
| ATOM | 561 | CD2 | PHE | A | 262 | 20.021 | 31.666 | 1.762 | 1.00 | 21.35 | C |
| ATOM | 562 | CE1 | PHE | A | 262 | 22.113 | 33.143 | 2.862 | 1.00 | 22.16 | C |
| ATOM | 563 | CE2 | PHE | A | 262 | 21.131 | 31.007 | 2.286 | 1.00 | 24.23 | C |
| ATOM | 564 | CZ | PHE | A | 262 | 22.187 | 31.749 | 2.843 | 1.00 | 24.05 | C |
| ATOM | 565 | N | LEU | A | 263 | 19.433 | 33.359 | −1.693 | 1.00 | 21.81 | N |
| ATOM | 566 | CA | LEU | A | 263 | 20.255 | 32.798 | −2.760 | 1.00 | 21.71 | C |
| ATOM | 567 | C | LEU | A | 263 | 20.688 | 33.887 | −3.738 | 1.00 | 21.65 | C |
| ATOM | 568 | O | LEU | A | 263 | 21.726 | 33.771 | −4.373 | 1.00 | 22.27 | O |
| ATOM | 569 | CB | LEU | A | 263 | 19.492 | 31.697 | −3.513 | 1.00 | 24.19 | C |
| ATOM | 570 | CG | LEU | A | 263 | 19.207 | 30.397 | −2.743 | 1.00 | 24.08 | C |
| ATOM | 571 | CD1 | LEU | A | 263 | 18.333 | 29.481 | −3.561 | 1.00 | 22.03 | C |
| ATOM | 572 | CD2 | LEU | A | 263 | 20.519 | 29.718 | −2.414 | 1.00 | 25.80 | C |
| ATOM | 573 | N | ASP | A | 264 | 19.892 | 34.945 | −3.856 | 1.00 | 23.16 | N |
| ATOM | 574 | CA | ASP | A | 264 | 20.229 | 36.035 | −4.757 | 1.00 | 23.40 | C |
| ATOM | 575 | C | ASP | A | 264 | 21.457 | 36.813 | −4.316 | 1.00 | 23.15 | C |
| ATOM | 576 | O | ASP | A | 264 | 22.184 | 37.332 | −5.153 | 1.00 | 23.44 | O |
| ATOM | 577 | CB | ASP | A | 264 | 19.068 | 37.034 | −4.889 | 1.00 | 25.31 | C |
| ATOM | 578 | CG | ASP | A | 264 | 17.867 | 36.456 | −5.616 | 1.00 | 29.79 | C |
| ATOM | 579 | OD1 | ASP | A | 264 | 18.066 | 35.647 | −6.550 | 1.00 | 25.94 | O |
| ATOM | 580 | OD2 | ASP | A | 264 | 16.716 | 36.825 | −5.257 | 1.00 | 32.01 | O |
| ATOM | 581 | N | VAL | A | 265 | 21.697 | 36.903 | −3.010 | 1.00 | 22.29 | N |
| ATOM | 582 | CA | VAL | A | 265 | 22.830 | 37.688 | −2.541 | 1.00 | 22.40 | C |
| ATOM | 583 | C | VAL | A | 265 | 24.126 | 36.930 | −2.491 | 1.00 | 24.29 | C |
| ATOM | 584 | O | VAL | A | 265 | 25.209 | 37.514 | −2.543 | 1.00 | 21.62 | O |
| ATOM | 585 | CB | VAL | A | 265 | 22.554 | 38.309 | −1.160 | 1.00 | 22.78 | C |
| ATOM | 586 | CG1 | VAL | A | 265 | 21.361 | 39.234 | −1.248 | 1.00 | 22.17 | C |
| ATOM | 587 | CG2 | VAL | A | 265 | 22.303 | 37.212 | −0.125 | 1.00 | 25.01 | C |
| ATOM | 588 | N | ILE | A | 266 | 24.024 | 35.618 | −2.395 | 1.00 | 28.03 | N |
| ATOM | 589 | CA | ILE | A | 266 | 25.219 | 34.815 | −2.335 | 1.00 | 32.30 | C |
| ATOM | 590 | C | ILE | A | 266 | 26.085 | 34.988 | −3.575 | 1.00 | 36.65 | C |
| ATOM | 591 | O | ILE | A | 266 | 27.290 | 35.182 | −3.462 | 1.00 | 33.84 | O |
| ATOM | 592 | CB | ILE | A | 266 | 24.866 | 33.354 | −2.183 | 1.00 | 32.52 | C |
| ATOM | 593 | CG1 | ILE | A | 266 | 23.992 | 33.176 | −0.941 | 1.00 | 30.68 | C |
| ATOM | 594 | CG2 | ILE | A | 266 | 26.146 | 32.527 | −2.112 | 1.00 | 32.27 | C |
| ATOM | 595 | CD1 | ILE | A | 266 | 23.468 | 31.773 | −0.779 | 1.00 | 29.89 | C |
| ATOM | 596 | N | PRO | A | 267 | 25.472 | 34.942 | −4.777 | 1.00 | 42.01 | N |
| ATOM | 597 | CA | PRO | A | 267 | 26.183 | 35.085 | −6.047 | 1.00 | 46.40 | C |
| ATOM | 598 | C | PRO | A | 267 | 27.331 | 36.069 | −6.062 | 1.00 | 50.69 | C |
| ATOM | 599 | O | PRO | A | 267 | 27.145 | 37.274 | −6.218 | 1.00 | 51.98 | O |
| ATOM | 600 | CB | PRO | A | 267 | 25.071 | 35.461 | −7.015 | 1.00 | 44.71 | C |
| ATOM | 601 | CG | PRO | A | 267 | 23.961 | 34.622 | −6.527 | 1.00 | 43.95 | C |
| ATOM | 602 | CD | PRO | A | 267 | 24.021 | 34.874 | −5.030 | 1.00 | 43.28 | C |
| ATOM | 603 | N | PHE | A | 268 | 28.526 | 35.519 | −5.902 | 1.00 | 55.60 | N |
| ATOM | 604 | CA | PHE | A | 268 | 29.759 | 36.281 | −5.906 | 1.00 | 59.19 | C |
| ATOM | 605 | C | PHE | A | 268 | 29.694 | 37.604 | −5.172 | 1.00 | 60.40 | C |
| ATOM | 606 | O | PHE | A | 268 | 30.654 | 38.374 | −5.219 | 1.00 | 62.57 | O |
| ATOM | 607 | CB | PHE | A | 268 | 30.234 | 36.521 | −7.342 | 1.00 | 61.53 | C |
| ATOM | 608 | CG | PHE | A | 268 | 30.380 | 35.265 | −8.149 | 1.00 | 64.38 | C |
| ATOM | 609 | CD1 | PHE | A | 268 | 29.259 | 34.573 | −8.596 | 1.00 | 65.44 | C |
| ATOM | 610 | CD2 | PHE | A | 268 | 31.641 | 34.765 | −8.455 | 1.00 | 67.03 | C |
| ATOM | 611 | CE1 | PHE | A | 268 | 29.389 | 33.401 | −9.336 | 1.00 | 66.75 | C |
| ATOM | 612 | CE2 | PHE | A | 268 | 31.785 | 33.593 | −9.194 | 1.00 | 68.08 | C |
| ATOM | 613 | CZ | PHE | A | 268 | 30.654 | 32.909 | −9.636 | 1.00 | 67.42 | C |
| ATOM | 614 | N | TRP | A | 269 | 28.578 | 37.898 | −4.508 | 1.00 | 61.29 | N |
| ATOM | 615 | CA | TRP | A | 269 | 28.505 | 39.143 | −3.749 | 1.00 | 61.28 | C |
| ATOM | 616 | C | TRP | A | 269 | 29.329 | 38.879 | −2.510 | 1.00 | 60.98 | C |
| ATOM | 617 | O | TRP | A | 269 | 29.558 | 39.772 | −1.697 | 1.00 | 62.09 | O |
| ATOM | 618 | CB | TRP | A | 269 | 27.071 | 39.505 | −3.377 | 1.00 | 62.02 | C |
| ATOM | 619 | CG | TRP | A | 269 | 26.508 | 40.535 | −4.280 | 1.00 | 62.97 | C |
| ATOM | 620 | CD1 | TRP | A | 269 | 26.947 | 41.817 | −4.434 | 1.00 | 64.86 | C |
| ATOM | 621 | CD2 | TRP | A | 269 | 25.460 | 40.354 | −5.230 | 1.00 | 64.57 | C |
| ATOM | 622 | CE2 | TRP | A | 269 | 25.321 | 41.567 | −5.937 | 1.00 | 65.04 | C |
| ATOM | 623 | CE3 | TRP | A | 269 | 24.624 | 39.280 | −5.558 | 1.00 | 65.91 | C |
| ATOM | 624 | NE1 | TRP | A | 269 | 26.242 | 42.445 | −5.431 | 1.00 | 66.10 | N |
| ATOM | 625 | CZ2 | TRP | A | 269 | 24.382 | 41.737 | −6.955 | 1.00 | 65.30 | C |
| ATOM | 626 | CZ3 | TRP | A | 269 | 23.690 | 39.447 | −6.570 | 1.00 | 66.90 | C |
| ATOM | 627 | CH2 | TRP | A | 269 | 23.577 | 40.670 | −7.258 | 1.00 | 66.55 | C |
| ATOM | 628 | N | LYS | A | 270 | 29.759 | 37.622 | −2.399 | 1.00 | 60.49 | N |
| ATOM | 629 | CA | LYS | A | 270 | 30.613 | 37.129 | −1.330 | 1.00 | 60.13 | C |
| ATOM | 630 | C | LYS | A | 270 | 31.949 | 36.835 | −2.044 | 1.00 | 62.90 | C |
| ATOM | 631 | O | LYS | A | 270 | 32.388 | 37.672 | −2.831 | 1.00 | 64.97 | O |
| ATOM | 632 | CB | LYS | A | 270 | 30.010 | 35.865 | −0.703 | 1.00 | 56.41 | C |
| ATOM | 633 | CG | LYS | A | 270 | 28.523 | 35.969 | −0.340 | 1.00 | 51.44 | C |
| ATOM | 634 | CD | LYS | A | 270 | 28.209 | 37.164 | 0.548 | 1.00 | 47.13 | C |
| ATOM | 635 | CE | LYS | A | 270 | 26.709 | 37.343 | 0.734 | 1.00 | 42.57 | C |
| ATOM | 636 | NZ | LYS | A | 270 | 26.093 | 36.211 | 1.472 | 1.00 | 43.68 | N |
| ATOM | 637 | N | LEU | A | 271 | 32.591 | 35.679 | −1.835 | 1.00 | 65.19 | N |
| ATOM | 638 | CA | LEU | A | 271 | 33.886 | 35.427 | −2.509 | 1.00 | 66.57 | C |
| ATOM | 639 | C | LEU | A | 271 | 34.399 | 33.974 | −2.610 | 1.00 | 67.60 | C |
| ATOM | 640 | O | LEU | A | 271 | 33.784 | 33.034 | −2.110 | 1.00 | 68.48 | O |
| ATOM | 641 | CB | LEU | A | 271 | 34.992 | 36.256 | −1.825 | 1.00 | 67.72 | C |
| ATOM | 642 | CG | LEU | A | 271 | 35.031 | 37.795 | −1.770 | 1.00 | 68.00 | C |
| ATOM | 643 | CD1 | LEU | A | 271 | 36.069 | 38.249 | −0.749 | 1.00 | 67.80 | C |
| ATOM | 644 | CD2 | LEU | A | 271 | 35.363 | 38.360 | −3.139 | 1.00 | 68.91 | C |
| ATOM | 645 | N | ASP | A | 272 | 35.543 | 33.839 | −3.287 | 1.00 | 67.99 | N |
| ATOM | 646 | CA | ASP | A | 272 | 36.292 | 32.583 | −3.485 | 1.00 | 67.89 | C |
| ATOM | 647 | C | ASP | A | 272 | 35.901 | 31.460 | −4.456 | 1.00 | 67.73 | C |
| ATOM | 648 | O | ASP | A | 272 | 36.627 | 31.194 | −5.412 | 1.00 | 68.27 | O |
| ATOM | 649 | CB | ASP | A | 272 | 36.536 | 31.936 | −2.139 | 1.00 | 68.60 | C |
| ATOM | 650 | CG | ASP | A | 272 | 37.049 | 30.531 | −2.273 | 1.00 | 69.55 | C |
| ATOM | 651 | OD1 | ASP | A | 272 | 38.269 | 30.364 | −2.444 | 1.00 | 70.20 | O |
| ATOM | 652 | OD2 | ASP | A | 272 | 36.212 | 29.601 | −2.242 | 1.00 | 69.34 | O |
| ATOM | 653 | N | LEU | A | 273 | 34.815 | 30.749 | −4.167 | 1.00 | 66.26 | N |
| ATOM | 654 | CA | LEU | A | 273 | 34.344 | 29.661 | −5.031 | 1.00 | 64.26 | C |
| ATOM | 655 | C | LEU | A | 273 | 35.059 | 28.301 | −4.969 | 1.00 | 62.38 | C |
| ATOM | 656 | O | LEU | A | 273 | 34.415 | 27.267 | −5.175 | 1.00 | 62.72 | O |
| ATOM | 657 | CB | LEU | A | 273 | 34.283 | 30.131 | −6.492 | 1.00 | 64.46 | C |
| ATOM | 658 | CG | LEU | A | 273 | 33.005 | 30.846 | −6.956 | 1.00 | 65.15 | C |
| ATOM | 659 | CD1 | LEU | A | 273 | 31.808 | 29.926 | −6.733 | 1.00 | 65.46 | C |
| ATOM | 660 | CD2 | LEU | A | 273 | 32.814 | 32.153 | −6.202 | 1.00 | 65.77 | C |
| ATOM | 661 | N | ASP | A | 274 | 36.364 | 28.264 | −4.711 | 1.00 | 58.91 | N |
| ATOM | 662 | CA | ASP | A | 274 | 37.035 | 26.961 | −4.634 | 1.00 | 54.79 | C |
| ATOM | 663 | C | ASP | A | 274 | 37.467 | 26.611 | −3.214 | 1.00 | 52.39 | C |
| ATOM | 664 | O | ASP | A | 274 | 38.479 | 25.944 | −3.001 | 1.00 | 50.63 | O |
| ATOM | 665 | CB | ASP | A | 274 | 38.254 | 26.902 | −5.561 | 1.00 | 55.77 | C |
| ATOM | 666 | CG | ASP | A | 274 | 39.400 | 27.755 | −5.076 | 1.00 | 55.94 | C |
| ATOM | 667 | OD1 | ASP | A | 274 | 40.566 | 27.379 | −5.340 | 1.00 | 56.72 | O |
| ATOM | 668 | OD2 | ASP | A | 274 | 39.141 | 28.797 | −4.440 | 1.00 | 54.47 | O |
| ATOM | 669 | N | GLN | A | 275 | 36.688 | 27.058 | −2.238 | 1.00 | 49.16 | N |
| ATOM | 670 | CA | GLN | A | 275 | 37.007 | 26.781 | −0.848 | 1.00 | 45.74 | C |
| ATOM | 671 | C | GLN | A | 275 | 35.758 | 26.218 | −0.175 | 1.00 | 43.27 | C |
| ATOM | 672 | O | GLN | A | 275 | 34.657 | 26.329 | −0.719 | 1.00 | 43.21 | O |
| ATOM | 673 | CB | GLN | A | 275 | 37.446 | 28.066 | −0.150 | 1.00 | 46.24 | C |
| ATOM | 674 | CG | GLN | A | 275 | 38.293 | 27.825 | 1.063 | 1.00 | 49.67 | C |
| ATOM | 675 | CD | GLN | A | 275 | 39.638 | 27.238 | 0.700 | 1.00 | 49.47 | C |
| ATOM | 676 | NE2 | GLN | A | 275 | 40.703 | 27.929 | 1.078 | 1.00 | 49.84 | N |
| ATOM | 677 | OE1 | GLN | A | 275 | 39.720 | 26.174 | 0.080 | 1.00 | 51.51 | O |
| ATOM | 678 | N | ASP | A | 276 | 35.917 | 25.612 | 0.996 | 1.00 | 38.26 | N |
| ATOM | 679 | CA | ASP | A | 276 | 34.767 | 25.058 | 1.700 | 1.00 | 35.87 | C |
| ATOM | 680 | C | ASP | A | 276 | 34.197 | 26.064 | 2.704 | 1.00 | 34.99 | C |
| ATOM | 681 | O | ASP | A | 276 | 34.925 | 26.648 | 3.513 | 1.00 | 32.22 | O |
| ATOM | 682 | CB | ASP | A | 276 | 35.141 | 23.754 | 2.417 | 1.00 | 36.22 | C |
| ATOM | 683 | CG | ASP | A | 276 | 35.598 | 22.661 | 1.453 | 1.00 | 37.53 | C |
| ATOM | 684 | OD1 | ASP | A | 276 | 34.981 | 22.499 | 0.382 | 1.00 | 37.85 | O |
| ATOM | 685 | OD2 | ASP | A | 276 | 36.572 | 21.951 | 1.773 | 1.00 | 40.48 | O |
| ATOM | 686 | N | TYR | A | 277 | 32.887 | 26.274 | 2.650 | 1.00 | 32.47 | N |
| ATOM | 687 | CA | TYR | A | 277 | 32.268 | 27.219 | 3.563 | 1.00 | 29.61 | C |
| ATOM | 688 | C | TYR | A | 277 | 31.117 | 26.652 | 4.358 | 1.00 | 29.96 | C |
| ATOM | 689 | O | TYR | A | 277 | 30.440 | 25.710 | 3.939 | 1.00 | 29.96 | O |
| ATOM | 690 | CB | TYR | A | 277 | 31.763 | 28.452 | 2.809 | 1.00 | 29.10 | C |
| ATOM | 691 | CG | TYR | A | 277 | 32.835 | 29.289 | 2.158 | 1.00 | 27.37 | C |
| ATOM | 692 | CD1 | TYR | A | 277 | 33.309 | 28.983 | 0.880 | 1.00 | 27.98 | C |
| ATOM | 693 | CD2 | TYR | A | 277 | 33.352 | 30.413 | 2.806 | 1.00 | 27.81 | C |
| ATOM | 694 | CE1 | TYR | A | 277 | 34.273 | 29.785 | 0.255 | 1.00 | 28.17 | C |
| ATOM | 695 | CE2 | TYR | A | 277 | 34.313 | 31.221 | 2.195 | 1.00 | 28.67 | C |
| ATOM | 696 | CZ | TYR | A | 277 | 34.765 | 30.903 | 0.920 | 1.00 | 27.46 | C |
| ATOM | 697 | OH | TYR | A | 277 | 35.684 | 31.719 | 0.313 | 1.00 | 26.19 | O |
| ATOM | 698 | N | ARG | A | 278 | 30.899 | 27.247 | 5.520 | 1.00 | 28.65 | N |
| ATOM | 699 | CA | ARG | A | 278 | 29.806 | 26.852 | 6.380 | 1.00 | 27.96 | C |
| ATOM | 700 | C | ARG | A | 278 | 28.805 | 27.993 | 6.316 | 1.00 | 25.36 | C |
| ATOM | 701 | O | ARG | A | 278 | 29.143 | 29.144 | 6.614 | 1.00 | 22.65 | O |
| ATOM | 702 | CB | ARG | A | 278 | 30.287 | 26.690 | 7.818 | 1.00 | 30.93 | C |
| ATOM | 703 | CG | ARG | A | 278 | 29.393 | 25.805 | 8.635 | 1.00 | 35.41 | C |
| ATOM | 704 | CD | ARG | A | 278 | 29.491 | 26.161 | 10.086 | 1.00 | 41.28 | C |
| ATOM | 705 | NE | ARG | A | 278 | 28.943 | 25.109 | 10.927 | 1.00 | 45.70 | N |
| ATOM | 706 | CZ | ARG | A | 278 | 28.844 | 25.196 | 12.249 | 1.00 | 48.55 | C |
| ATOM | 707 | NH1 | ARG | A | 278 | 29.254 | 26.296 | 12.872 | 1.00 | 47.81 | N |
| ATOM | 708 | NH2 | ARG | A | 278 | 28.346 | 24.180 | 12.946 | 1.00 | 48.38 | N |
| ATOM | 709 | N | VAL | A | 279 | 27.578 | 27.688 | 5.920 | 1.00 | 23.28 | N |
| ATOM | 710 | CA | VAL | A | 279 | 26.550 | 28.721 | 5.835 | 1.00 | 22.63 | C |
| ATOM | 711 | C | VAL | A | 279 | 25.436 | 28.347 | 6.794 | 1.00 | 21.54 | C |
| ATOM | 712 | O | VAL | A | 279 | 24.904 | 27.238 | 6.727 | 1.00 | 20.91 | O |
| ATOM | 713 | CB | VAL | A | 279 | 25.974 | 28.827 | 4.398 | 1.00 | 23.39 | C |
| ATOM | 714 | CG1 | VAL | A | 279 | 24.942 | 29.943 | 4.327 | 1.00 | 22.73 | C |
| ATOM | 715 | CG2 | VAL | A | 279 | 27.088 | 29.085 | 3.413 | 1.00 | 24.35 | C |
| ATOM | 716 | N | THR | A | 280 | 25.092 | 29.255 | 7.700 | 1.00 | 19.42 | N |
| ATOM | 717 | CA | THR | A | 280 | 24.031 | 28.969 | 8.672 | 1.00 | 19.95 | C |
| ATOM | 718 | C | THR | A | 280 | 22.897 | 29.975 | 8.485 | 1.00 | 18.85 | C |
| ATOM | 719 | O | THR | A | 280 | 23.146 | 31.154 | 8.286 | 1.00 | 18.42 | O |
| ATOM | 720 | CB | THR | A | 280 | 24.593 | 29.024 | 10.130 | 1.00 | 17.41 | C |
| ATOM | 721 | CG2 | THR | A | 280 | 23.542 | 28.623 | 11.145 | 1.00 | 21.21 | C |
| ATOM | 722 | OG1 | THR | A | 280 | 25.680 | 28.097 | 10.253 | 1.00 | 17.88 | O |
| ATOM | 723 | N | CYS | A | 281 | 21.652 | 29.522 | 8.509 | 1.00 | 19.74 | N |
| ATOM | 724 | CA | CYS | A | 281 | 20.549 | 30.477 | 8.366 | 1.00 | 21.71 | C |
| ATOM | 725 | C | CYS | A | 281 | 19.574 | 30.319 | 9.514 | 1.00 | 22.07 | C |
| ATOM | 726 | O | CYS | A | 281 | 19.276 | 29.201 | 9.936 | 1.00 | 24.85 | O |
| ATOM | 727 | CB | CYS | A | 281 | 19.772 | 30.267 | 7.058 | 1.00 | 20.80 | C |
| ATOM | 728 | SG | CYS | A | 281 | 20.653 | 30.526 | 5.512 | 1.00 | 27.86 | S |
| ATOM | 729 | N | PHE | A | 282 | 19.093 | 31.440 | 10.035 | 1.00 | 21.70 | N |
| ATOM | 730 | CA | PHE | A | 282 | 18.093 | 31.412 | 11.089 | 1.00 | 19.78 | C |
| ATOM | 731 | C | PHE | A | 282 | 16.888 | 32.065 | 10.424 | 1.00 | 19.98 | C |
| ATOM | 732 | O | PHE | A | 282 | 16.942 | 33.235 | 10.021 | 1.00 | 18.46 | O |
| ATOM | 733 | CB | PHE | A | 282 | 18.542 | 32.211 | 12.311 | 1.00 | 19.67 | C |
| ATOM | 734 | CG | PHE | A | 282 | 19.874 | 31.764 | 12.866 | 1.00 | 22.99 | C |
| ATOM | 735 | CD1 | PHE | A | 282 | 21.064 | 32.247 | 12.324 | 1.00 | 21.43 | C |
| ATOM | 736 | CD2 | PHE | A | 282 | 19.937 | 30.836 | 13.905 | 1.00 | 20.42 | C |
| ATOM | 737 | CE1 | PHE | A | 282 | 22.301 | 31.810 | 12.808 | 1.00 | 23.94 | C |
| ATOM | 738 | CE2 | PHE | A | 282 | 21.171 | 30.391 | 14.398 | 1.00 | 22.62 | C |
| ATOM | 739 | CZ | PHE | A | 282 | 22.354 | 30.877 | 13.849 | 1.00 | 23.16 | C |
| ATOM | 740 | N | THR | A | 283 | 15.820 | 31.295 | 10.277 | 1.00 | 16.66 | N |
| ATOM | 741 | CA | THR | A | 283 | 14.613 | 31.784 | 9.626 | 1.00 | 17.62 | C |
| ATOM | 742 | C | THR | A | 283 | 13.439 | 31.738 | 10.583 | 1.00 | 16.88 | C |
| ATOM | 743 | O | THR | A | 283 | 13.374 | 30.864 | 11.441 | 1.00 | 16.38 | O |
| ATOM | 744 | CB | THR | A | 283 | 14.287 | 30.932 | 8.385 | 1.00 | 17.15 | C |
| ATOM | 745 | CG2 | THR | A | 283 | 15.438 | 30.985 | 7.398 | 1.00 | 18.76 | C |
| ATOM | 746 | OG1 | THR | A | 283 | 14.091 | 29.564 | 8.781 | 1.00 | 17.85 | O |
| ATOM | 747 | N | SER | A | 284 | 12.520 | 32.688 | 10.448 | 1.00 | 18.70 | N |
| ATOM | 748 | CA | SER | A | 284 | 11.363 | 32.718 | 11.331 | 1.00 | 19.40 | C |
| ATOM | 749 | C | SER | A | 284 | 10.373 | 31.632 | 10.932 | 1.00 | 20.71 | C |
| ATOM | 750 | O | SER | A | 284 | 9.663 | 31.093 | 11.781 | 1.00 | 21.12 | O |
| ATOM | 751 | CB | SER | A | 284 | 10.719 | 34.109 | 11.314 | 1.00 | 20.52 | C |
| ATOM | 752 | OG | SER | A | 284 | 10.372 | 34.521 | 10.007 | 1.00 | 24.12 | O |
| ATOM | 753 | N | TRP | A | 285 | 10.349 | 31.308 | 9.635 | 1.00 | 20.91 | N |
| ATOM | 754 | CA | TRP | A | 285 | 9.494 | 30.253 | 9.070 | 1.00 | 19.31 | C |
| ATOM | 755 | C | TRP | A | 285 | 10.419 | 29.343 | 8.258 | 1.00 | 21.83 | C |
| ATOM | 756 | O | TRP | A | 285 | 11.403 | 29.825 | 7.684 | 1.00 | 20.98 | O |
| ATOM | 757 | CB | TRP | A | 285 | 8.459 | 30.825 | 8.090 | 1.00 | 22.60 | C |
| ATOM | 758 | CG | TRP | A | 285 | 7.236 | 31.448 | 8.699 | 1.00 | 22.00 | C |
| ATOM | 759 | CD1 | TRP | A | 285 | 6.878 | 32.764 | 8.655 | 1.00 | 20.85 | C |
| ATOM | 760 | CD2 | TRP | A | 285 | 6.220 | 30.779 | 9.445 | 1.00 | 21.57 | C |
| ATOM | 761 | CE2 | TRP | A | 285 | 5.281 | 31.756 | 9.841 | 1.00 | 23.56 | C |
| ATOM | 762 | CE3 | TRP | A | 285 | 6.017 | 29.451 | 9.836 | 1.00 | 23.47 | C |
| ATOM | 763 | NE1 | TRP | A | 285 | 5.704 | 32.959 | 9.339 | 1.00 | 22.65 | N |
| ATOM | 764 | CZ2 | TRP | A | 285 | 4.147 | 31.441 | 10.592 | 1.00 | 22.97 | C |
| ATOM | 765 | CZ3 | TRP | A | 285 | 4.898 | 29.139 | 10.582 | 1.00 | 22.58 | C |
| ATOM | 766 | CH2 | TRP | A | 285 | 3.980 | 30.131 | 10.960 | 1.00 | 23.50 | C |
| ATOM | 767 | N | SER | A | 286 | 10.118 | 28.045 | 8.205 | 1.00 | 19.66 | N |
| ATOM | 768 | CA | SER | A | 286 | 10.924 | 27.131 | 7.410 | 1.00 | 19.40 | C |
| ATOM | 769 | C | SER | A | 286 | 10.536 | 27.382 | 5.941 | 1.00 | 21.52 | C |
| ATOM | 770 | O | SER | A | 286 | 9.477 | 27.967 | 5.662 | 1.00 | 20.62 | O |
| ATOM | 771 | CB | SER | A | 285 | 10.681 | 25.658 | 7.811 | 1.00 | 19.39 | C |
| ATOM | 772 | OG | SER | A | 286 | 9.319 | 25.272 | 7.732 | 1.00 | 15.77 | O |
| ATOM | 773 | N | PRO | A | 287 | 11.395 | 26.971 | 4.993 | 1.00 | 20.13 | N |
| ATOM | 774 | CA | PRO | A | 287 | 11.176 | 27.142 | 3.549 | 1.00 | 22.26 | C |
| ATOM | 775 | C | PRO | A | 287 | 9.969 | 26.437 | 2.927 | 1.00 | 22.66 | C |
| ATOM | 776 | O | PRO | A | 287 | 9.628 | 25.325 | 3.316 | 1.00 | 21.23 | O |
| ATOM | 777 | CB | PRO | A | 287 | 12.497 | 26.665 | 2.935 | 1.00 | 19.86 | C |
| ATOM | 778 | CG | PRO | A | 287 | 13.019 | 25.704 | 3.934 | 1.00 | 22.83 | C |
| ATOM | 779 | CD | PRO | A | 287 | 12.700 | 26.342 | 5.260 | 1.00 | 21.65 | C |
| ATOM | 780 | N | CYS | A | 288 | 9.327 | 27.088 | 1.956 | 1.00 | 25.38 | N |
| ATOM | 781 | CA | CYS | A | 288 | 8.183 | 26.476 | 1.276 | 1.00 | 25.41 | C |
| ATOM | 782 | C | CYS | A | 288 | 8.732 | 25.414 | 0.311 | 1.00 | 25.60 | C |
| ATOM | 783 | O | CYS | A | 288 | 9.933 | 25.382 | 0.019 | 1.00 | 22.68 | O |
| ATOM | 784 | CB | CYS | A | 288 | 7.391 | 27.524 | 0.477 | 1.00 | 21.78 | C |
| ATOM | 785 | SG | CYS | A | 288 | 8.064 | 27.838 | −1.170 | 1.00 | 25.00 | S |
| ATOM | 786 | N | PHE | A | 289 | 7.842 | 24.566 | −0.193 | 1.00 | 26.65 | N |
| ATOM | 787 | CA | PHE | A | 289 | 8.216 | 23.495 | −1.112 | 1.00 | 29.22 | C |
| ATOM | 788 | C | PHE | A | 289 | 9.095 | 23.958 | −2.272 | 1.00 | 28.76 | C |
| ATOM | 789 | O | PHE | A | 289 | 10.081 | 23.302 | −2.615 | 1.00 | 27.96 | O |
| ATOM | 790 | CB | PHE | A | 289 | 6.945 | 22.829 | −1.651 | 1.00 | 34.58 | C |
| ATOM | 791 | CG | PHE | A | 289 | 5.845 | 23.807 | −1.969 | 1.00 | 40.95 | C |
| ATOM | 792 | CD1 | PHE | A | 289 | 5.818 | 24.481 | −3.192 | 1.00 | 41.32 | C |
| ATOM | 793 | CD2 | PHE | A | 289 | 4.862 | 24.099 | −1.019 | 1.00 | 42.23 | C |
| ATOM | 794 | CE1 | PHE | A | 289 | 4.832 | 25.432 | −3.462 | 1.00 | 44.01 | C |
| ATOM | 795 | CE2 | PHE | A | 289 | 3.872 | 25.048 | −1.280 | 1.00 | 44.03 | C |
| ATOM | 796 | CZ | PHE | A | 289 | 3.856 | 25.718 | −2.504 | 1.00 | 43.64 | C |
| ATOM | 797 | N | SER | A | 290 | 8.742 | 25.092 | −2.866 | 1.00 | 26.95 | N |
| ATOM | 798 | CA | SER | A | 290 | 9.486 | 25.626 | −4.000 | 1.00 | 26.41 | C |
| ATOM | 799 | C | SER | A | 290 | 10.863 | 26.154 | −3.603 | 1.00 | 25.54 | C |
| ATOM | 800 | O | SER | A | 290 | 11.853 | 25.931 | −4.300 | 1.00 | 24.73 | O |
| ATOM | 801 | CB | SER | A | 290 | 8.674 | 26.732 | −4.676 | 1.00 | 27.61 | C |
| ATOM | 802 | OG | SER | A | 290 | 9.407 | 27.321 | −5.732 | 1.00 | 34.27 | O |
| ATOM | 803 | N | CYS | A | 291 | 10.934 | 26.861 | −2.485 | 1.00 | 24.35 | N |
| ATOM | 804 | CA | CYS | A | 291 | 12.218 | 27.376 | −2.048 | 1.00 | 24.41 | C |
| ATOM | 805 | C | CYS | A | 291 | 13.087 | 26.248 | −1.481 | 1.00 | 24.43 | C |
| ATOM | 806 | O | CYS | A | 291 | 14.314 | 26.350 | −1.455 | 1.00 | 25.47 | O |
| ATOM | 807 | CB | CYS | A | 291 | 12.017 | 28.497 | −1.023 | 1.00 | 23.01 | C |
| ATOM | 808 | SG | CYS | A | 291 | 11.286 | 30.014 | −1.753 | 1.00 | 21.56 | S |
| ATOM | 809 | N | ALA | A | 292 | 12.464 | 25.161 | −1.036 | 1.00 | 23.71 | N |
| ATOM | 810 | CA | ALA | A | 292 | 13.252 | 24.052 | −0.510 | 1.00 | 23.37 | C |
| ATOM | 811 | C | ALA | A | 292 | 14.030 | 23.370 | −1.635 | 1.00 | 23.33 | C |
| ATOM | 812 | O | ALA | A | 292 | 15.216 | 23.052 | −1.481 | 1.00 | 21.45 | O |
| ATOM | 813 | CB | ALA | A | 292 | 12.361 | 23.046 | 0.197 | 1.00 | 24.50 | C |
| ATOM | 814 | N | GLN | A | 293 | 13.377 | 23.153 | −2.774 | 1.00 | 23.64 | N |
| ATOM | 815 | CA | GLN | A | 293 | 14.059 | 22.500 | −3.888 | 1.00 | 25.62 | C |
| ATOM | 816 | C | GLN | A | 293 | 15.148 | 23.406 | −4.451 | 1.00 | 23.77 | C |
| ATOM | 817 | O | GLN | A | 293 | 16.193 | 22.932 | −4.889 | 1.00 | 23.39 | O |
| ATOM | 818 | CB | GLN | A | 293 | 13.066 | 22.102 | −4.985 | 1.00 | 29.80 | C |
| ATOM | 819 | CG | GLN | A | 293 | 12.264 | 23.247 | −5.573 | 1.00 | 35.63 | C |
| ATOM | 820 | CD | GLN | A | 293 | 11.351 | 22.810 | −6.720 | 1.00 | 39.34 | C |
| ATOM | 821 | NE2 | GLN | A | 293 | 11.235 | 21.496 | −6.925 | 1.00 | 38.32 | N |
| ATOM | 822 | OE1 | GLN | A | 293 | 10.761 | 23.651 | −7.414 | 1.00 | 40.93 | O |
| ATOM | 823 | N | GLU | A | 294 | 14.903 | 24.712 | −4.407 | 1.00 | 22.40 | N |
| ATOM | 824 | CA | GLU | A | 294 | 15.859 | 25.692 | −4.896 | 1.00 | 20.90 | C |
| ATOM | 825 | C | GLU | A | 294 | 17.136 | 25.635 | −4.048 | 1.00 | 21.42 | C |
| ATOM | 826 | O | GLU | A | 294 | 18.242 | 25.605 | −4.589 | 1.00 | 21.49 | O |
| ATOM | 827 | CB | GLU | A | 294 | 15.236 | 27.083 | −4.831 | 1.00 | 22.46 | C |
| ATOM | 828 | CG | GLU | A | 294 | 15.662 | 28.011 | −5.942 | 1.00 | 28.96 | C |
| ATOM | 829 | CD | GLU | A | 294 | 15.461 | 27.390 | −7.316 | 1.00 | 31.72 | C |
| ATOM | 830 | OE1 | GLU | A | 294 | 14.341 | 26.908 | −7.596 | 1.00 | 31.50 | O |
| ATOM | 831 | OE2 | GLU | A | 294 | 16.428 | 27.384 | −8.112 | 1.00 | 33.05 | O |
| ATOM | 832 | N | MET | A | 295 | 16.987 | 25.623 | −2.721 | 1.00 | 18.96 | N |
| ATOM | 833 | CA | MET | A | 295 | 18.151 | 25.552 | −1.831 | 1.00 | 19.63 | C |
| ATOM | 834 | C | MET | A | 295 | 18.849 | 24.184 | −1.971 | 1.00 | 20.95 | C |
| ATOM | 835 | O | MET | A | 295 | 20.083 | 24.093 | −1.945 | 1.00 | 21.08 | O |
| ATOM | 836 | CB | MET | A | 295 | 17.733 | 25.796 | −0.370 | 1.00 | 19.64 | C |
| ATOM | 837 | CG | MET | A | 295 | 17.000 | 27.130 | −0.132 | 1.00 | 20.46 | C |
| ATOM | 838 | SD | MET | A | 295 | 16.356 | 27.358 | 1.563 | 1.00 | 20.73 | S |
| ATOM | 839 | CE | MET | A | 295 | 17.516 | 28.593 | 2.148 | 1.00 | 23.49 | C |
| ATOM | 840 | N | ALA | A | 296 | 18.056 | 23.129 | −2.139 | 1.00 | 20.57 | N |
| ATOM | 841 | CA | ALA | A | 296 | 18.588 | 21.776 | −2.292 | 1.00 | 23.12 | C |
| ATOM | 842 | C | ALA | A | 296 | 19.420 | 21.735 | −3.566 | 1.00 | 24.84 | C |
| ATOM | 843 | O | ALA | A | 296 | 20.454 | 21.066 | −3.639 | 1.00 | 25.35 | O |
| ATOM | 844 | CB | ALA | A | 296 | 17.437 | 20.761 | −2.374 | 1.00 | 21.44 | C |
| ATOM | 845 | N | LYS | A | 297 | 18.952 | 22.468 | −4.570 | 1.00 | 25.94 | N |
| ATOM | 846 | CA | LYS | A | 297 | 19.637 | 22.566 | −5.850 | 1.00 | 25.93 | C |
| ATOM | 847 | C | LYS | A | 297 | 21.000 | 23.211 | −5.624 | 1.00 | 25.91 | C |
| ATOM | 848 | O | LYS | A | 297 | 22.043 | 22.681 | −6.031 | 1.00 | 24.18 | O |
| ATOM | 849 | CB | LYS | A | 297 | 18.821 | 23.433 | −6.807 | 1.00 | 28.51 | C |
| ATOM | 850 | CG | LYS | A | 297 | 19.373 | 23.506 | −8.216 | 1.00 | 31.05 | C |
| ATOM | 851 | CD | LYS | A | 297 | 18.485 | 24.340 | −9.129 | 1.00 | 32.07 | C |
| ATOM | 852 | CE | LYS | A | 297 | 17.229 | 23.599 | −9.533 | 1.00 | 30.37 | C |
| ATOM | 853 | NZ | LYS | A | 297 | 16.383 | 23.283 | −8.373 | 1.00 | 29.36 | N |
| ATOM | 854 | N | PHE | A | 298 | 20.981 | 24.364 | −4.968 | 1.00 | 24.75 | N |
| ATOM | 855 | CA | PHE | A | 298 | 22.198 | 25.101 | −4.687 | 1.00 | 25.86 | C |
| ATOM | 856 | C | PHE | A | 298 | 23.280 | 24.255 | −4.023 | 1.00 | 26.05 | C |
| ATOM | 857 | O | PHE | A | 298 | 24.382 | 24.115 | −4.558 | 1.00 | 25.17 | O |
| ATOM | 858 | CB | PHE | A | 298 | 21.895 | 26.313 | −3.806 | 1.00 | 27.15 | C |
| ATOM | 859 | CG | PHE | A | 298 | 23.120 | 26.992 | −3.286 | 1.00 | 29.83 | C |
| ATOM | 860 | CD1 | PHE | A | 298 | 23.993 | 27.635 | −4.152 | 1.00 | 32.89 | C |
| ATOM | 861 | CD2 | PHE | A | 298 | 23.411 | 26.979 | −1.928 | 1.00 | 31.79 | C |
| ATOM | 862 | CE1 | PHE | A | 298 | 25.148 | 28.262 | −3.677 | 1.00 | 33.59 | C |
| ATOM | 863 | CE2 | PHE | A | 298 | 24.560 | 27.603 | −1.437 | 1.00 | 33.54 | C |
| ATOM | 864 | CZ | PHE | A | 298 | 25.429 | 28.245 | −2.314 | 1.00 | 33.44 | C |
| ATOM | 865 | N | ILE | A | 299 | 22.978 | 23.687 | −2.862 | 1.00 | 27.68 | N |
| ATOM | 866 | CA | ILE | A | 299 | 23.981 | 22.886 | −2.169 | 1.00 | 29.91 | C |
| ATOM | 867 | C | ILE | A | 299 | 24.388 | 21.654 | −2.947 | 1.00 | 32.21 | C |
| ATOM | 868 | O | ILE | A | 299 | 25.501 | 21.156 | −2.781 | 1.00 | 33.65 | O |
| ATOM | 869 | CB | ILE | A | 299 | 23.506 | 22.447 | −0.777 | 1.00 | 29.48 | C |
| ATOM | 870 | CG1 | ILE | A | 299 | 22.237 | 21.606 | −0.895 | 1.00 | 30.52 | C |
| ATOM | 871 | CG2 | ILE | A | 299 | 23.277 | 23.667 | 0.094 | 1.00 | 29.71 | C |
| ATOM | 872 | CD1 | ILE | A | 299 | 21.685 | 21.177 | 0.437 | 1.00 | 31.02 | C |
| ATOM | 873 | N | SER | A | 300 | 23.497 | 21.161 | −3.799 | 1.00 | 33.90 | N |
| ATOM | 874 | CA | SER | A | 300 | 23.804 | 19.975 | −4.593 | 1.00 | 36.21 | C |
| ATOM | 875 | C | SER | A | 300 | 24.813 | 20.273 | −5.692 | 1.00 | 38.66 | C |
| ATOM | 876 | O | SER | A | 300 | 25.679 | 19.452 | −5.990 | 1.00 | 38.48 | O |
| ATOM | 877 | CB | SER | A | 300 | 22.530 | 19.406 | −5.225 | 1.00 | 34.65 | C |
| ATOM | 878 | OG | SER | A | 300 | 21.704 | 18.796 | −4.249 | 1.00 | 34.99 | O |
| ATOM | 879 | N | LYS | A | 301 | 24.701 | 21.451 | −6.293 | 1.00 | 41.96 | N |
| ATOM | 880 | CA | LYS | A | 301 | 25.598 | 21.826 | −7.375 | 1.00 | 45.68 | C |
| ATOM | 881 | C | LYS | A | 301 | 26.846 | 22.564 | −6.916 | 1.00 | 46.28 | C |
| ATOM | 882 | O | LYS | A | 301 | 27.650 | 22.987 | −7.736 | 1.00 | 47.17 | O |
| ATOM | 883 | CB | LYS | A | 301 | 24.851 | 22.683 | −8.397 | 1.00 | 48.42 | C |
| ATOM | 884 | CG | LYS | A | 301 | 23.685 | 21.968 | −9.068 | 1.00 | 53.12 | C |
| ATOM | 885 | CD | LYS | A | 301 | 23.125 | 22.797 | −10.220 | 1.00 | 56.14 | C |
| ATOM | 886 | CE | LYS | A | 301 | 21.948 | 22.106 | −10.894 | 1.00 | 56.57 | C |
| ATOM | 887 | NZ | LYS | A | 301 | 21.424 | 22.901 | −12.038 | 1.00 | 58.21 | N |
| ATOM | 888 | N | ASN | A | 302 | 27.016 | 22.713 | −5.609 | 1.00 | 47.96 | N |
| ATOM | 889 | CA | ASN | A | 302 | 28.175 | 23.415 | −5.096 | 1.00 | 49.85 | C |
| ATOM | 890 | C | ASN | A | 302 | 29.169 | 22.552 | −4.321 | 1.00 | 50.59 | C |
| ATOM | 891 | O | ASN | A | 302 | 30.379 | 22.666 | −4.520 | 1.00 | 52.62 | O |
| ATOM | 892 | CB | ASN | A | 302 | 27.719 | 24.599 | −4.254 | 1.00 | 51.58 | C |
| ATOM | 893 | CG | ASN | A | 302 | 27.287 | 25.775 | −5.105 | 1.00 | 54.30 | C |
| ATOM | 894 | ND2 | ASN | A | 302 | 27.982 | 26.899 | −4.947 | 1.00 | 55.57 | N |
| ATOM | 895 | OD1 | ASN | A | 302 | 26.350 | 25.679 | −5.905 | 1.00 | 54.58 | O |
| ATOM | 896 | N | LYS | A | 303 | 28.675 | 21.697 | −3.436 | 1.00 | 50.59 | N |
| ATOM | 897 | CA | LYS | A | 303 | 29.546 | 20.812 | −2.664 | 1.00 | 49.87 | C |
| ATOM | 898 | C | LYS | A | 303 | 30.437 | 21.571 | −1.669 | 1.00 | 48.46 | C |
| ATOM | 899 | O | LYS | A | 303 | 30.607 | 21.139 | −0.527 | 1.00 | 47.86 | O |
| ATOM | 900 | CB | LYS | A | 303 | 30.408 | 19.981 | −3.622 | 1.00 | 51.68 | C |
| ATOM | 901 | CG | LYS | A | 303 | 31.072 | 18.772 | −2.992 | 1.00 | 53.29 | C |
| ATOM | 902 | CD | LYS | A | 303 | 31.880 | 17.971 | −4.011 | 1.00 | 55.72 | C |
| ATOM | 903 | CE | LYS | A | 303 | 31.016 | 17.449 | −5.158 | 1.00 | 56.75 | C |
| ATOM | 904 | NZ | LYS | A | 303 | 31.809 | 16.595 | −6.096 | 1.00 | 55.79 | N |
| ATOM | 905 | N | HIS | A | 304 | 30.998 | 22.698 | −2.098 | 1.00 | 45.99 | N |
| ATOM | 906 | CA | HIS | A | 304 | 31.856 | 23.494 | −1.228 | 1.00 | 44.40 | C |
| ATOM | 907 | C | HIS | A | 304 | 31.079 | 24.395 | −0.278 | 1.00 | 41.88 | C |
| ATOM | 908 | O | HIS | A | 304 | 31.612 | 25.397 | 0.189 | 1.00 | 44.02 | O |
| ATOM | 909 | CB | HIS | A | 304 | 32.811 | 24.363 | −2.051 | 1.00 | 45.42 | C |
| ATOM | 910 | CG | HIS | A | 304 | 33.755 | 23.580 | −2.905 | 1.00 | 47.39 | C |
| ATOM | 911 | CD2 | HIS | A | 304 | 34.073 | 23.695 | −4.215 | 1.00 | 47.67 | C |
| ATOM | 912 | ND1 | HIS | A | 304 | 34.489 | 22.520 | −2.420 | 1.00 | 48.44 | N |
| ATOM | 913 | CE1 | HIS | A | 304 | 35.217 | 22.010 | −3.399 | 1.00 | 49.46 | C |
| ATOM | 914 | NE2 | HIS | A | 304 | 34.982 | 22.705 | −4.497 | 1.00 | 48.97 | N |
| ATOM | 915 | N | VAL | A | 305 | 29.827 | 24.049 | 0.004 | 1.00 | 39.14 | N |
| ATOM | 916 | CA | VAL | A | 305 | 29.002 | 24.845 | 0.913 | 1.00 | 35.54 | C |
| ATOM | 917 | C | VAL | A | 305 | 28.156 | 23.972 | 1.833 | 1.00 | 33.83 | C |
| ATOM | 918 | O | VAL | A | 305 | 27.273 | 23.244 | 1.367 | 1.00 | 32.85 | O |
| ATOM | 919 | CB | VAL | A | 305 | 28.039 | 25.800 | 0.148 | 1.00 | 36.45 | C |
| ATOM | 920 | CG1 | VAL | A | 305 | 27.022 | 26.398 | 1.113 | 1.00 | 33.70 | C |
| ATOM | 921 | CG2 | VAL | A | 305 | 28.823 | 26.924 | −0.530 | 1.00 | 33.94 | C |
| ATOM | 922 | N | SER | A | 306 | 28.442 | 24.053 | 3.135 | 1.00 | 29.90 | N |
| ATOM | 923 | CA | SER | A | 306 | 27.708 | 23.314 | 4.156 | 1.00 | 28.77 | C |
| ATOM | 924 | C | SER | A | 306 | 26.593 | 24.225 | 4.658 | 1.00 | 24.66 | C |
| ATOM | 925 | O | SER | A | 306 | 26.864 | 25.219 | 5.315 | 1.00 | 25.00 | O |
| ATOM | 926 | CB | SER | A | 306 | 28.600 | 22.967 | 5.360 | 1.00 | 31.36 | C |
| ATOM | 927 | OG | SER | A | 306 | 29.747 | 22.234 | 4.988 | 1.00 | 35.68 | O |
| ATOM | 928 | N | LEU | A | 307 | 25.353 | 23.863 | 4.367 | 1.00 | 23.71 | N |
| ATOM | 929 | CA | LEU | A | 307 | 24.190 | 24.642 | 4.768 | 1.00 | 20.81 | C |
| ATOM | 930 | C | LEU | A | 307 | 23.474 | 24.078 | 6.001 | 1.00 | 20.26 | C |
| ATOM | 931 | O | LEU | A | 307 | 23.071 | 22.906 | 6.027 | 1.00 | 19.88 | O |
| ATOM | 932 | CB | LEU | A | 307 | 23.201 | 24.711 | 3.598 | 1.00 | 20.72 | C |
| ATOM | 933 | CG | LEU | A | 307 | 21.895 | 25.486 | 3.829 | 1.00 | 21.02 | C |
| ATOM | 934 | CD1 | LEU | A | 307 | 22.223 | 26.947 | 4.120 | 1.00 | 21.84 | C |
| ATOM | 935 | CD2 | LEU | A | 307 | 20.988 | 25.365 | 2.601 | 1.00 | 19.75 | C |
| ATOM | 936 | N | CYS | A | 308 | 23.328 | 24.924 | 7.014 | 1.00 | 17.92 | N |
| ATOM | 937 | CA | CYS | A | 308 | 22.639 | 24.574 | 8.246 | 1.00 | 22.08 | C |
| ATOM | 938 | C | CYS | A | 308 | 21.501 | 25.579 | 8.397 | 1.00 | 20.55 | C |
| ATOM | 939 | O | CYS | A | 308 | 21.740 | 26.783 | 8.532 | 1.00 | 22.47 | O |
| ATOM | 940 | CB | CYS | A | 308 | 23.578 | 24.669 | 9.451 | 1.00 | 23.43 | C |
| ATOM | 941 | SG | CYS | A | 308 | 24.998 | 23.554 | 9.349 | 1.00 | 35.24 | S |
| ATOM | 942 | N | ILE | A | 309 | 20.270 | 25.085 | 8.355 | 1.00 | 19.19 | N |
| ATOM | 943 | CA | ILE | A | 309 | 19.097 | 25.945 | 8.469 | 1.00 | 19.87 | C |
| ATOM | 944 | C | ILE | A | 309 | 18.397 | 25.746 | 9.799 | 1.00 | 19.98 | C |
| ATOM | 945 | O | ILE | A | 309 | 17.934 | 24.649 | 10.088 | 1.00 | 22.77 | O |
| ATOM | 946 | CB | ILE | A | 309 | 18.070 | 25.632 | 7.356 | 1.00 | 21.02 | C |
| ATOM | 947 | CG1 | ILE | A | 309 | 18.708 | 25.822 | 5.985 | 1.00 | 23.06 | C |
| ATOM | 948 | CG2 | ILE | A | 309 | 16.849 | 26.520 | 7.500 | 1.00 | 22.27 | C |
| ATOM | 949 | CD1 | ILE | A | 309 | 18.293 | 24.749 | 4.999 | 1.00 | 22.94 | C |
| ATOM | 950 | N | PHE | A | 310 | 18.336 | 26.800 | 10.607 | 1.00 | 19.52 | N |
| ATOM | 951 | CA | PHE | A | 310 | 17.647 | 26.757 | 11.887 | 1.00 | 19.16 | C |
| ATOM | 952 | C | PHE | A | 310 | 16.384 | 27.593 | 11.692 | 1.00 | 19.92 | C |
| ATOM | 953 | O | PHE | A | 310 | 16.483 | 28.742 | 11.287 | 1.00 | 17.33 | O |
| ATOM | 954 | CB | PHE | A | 310 | 18.497 | 27.372 | 13.013 | 1.00 | 21.13 | C |
| ATOM | 955 | CG | PHE | A | 310 | 19.695 | 26.548 | 13.395 | 1.00 | 20.02 | C |
| ATOM | 956 | CD1 | PHE | A | 310 | 20.902 | 26.688 | 12.718 | 1.00 | 20.02 | C |
| ATOM | 957 | CD2 | PHE | A | 310 | 19.607 | 25.613 | 14.423 | 1.00 | 19.15 | C |
| ATOM | 958 | CE1 | PHE | A | 310 | 22.006 | 25.902 | 13.064 | 1.00 | 21.41 | C |
| ATOM | 959 | CE2 | PHE | A | 310 | 20.700 | 24.823 | 14.772 | 1.00 | 21.44 | C |
| ATOM | 960 | CZ | PHE | A | 310 | 21.905 | 24.965 | 14.092 | 1.00 | 19.47 | C |
| ATOM | 961 | N | THR | A | 311 | 15.212 | 27.011 | 11.963 | 1.00 | 18.96 | N |
| ATOM | 962 | CA | THR | A | 311 | 13.945 | 27.715 | 11.794 | 1.00 | 19.13 | C |
| ATOM | 963 | C | THR | A | 311 | 13.188 | 27.829 | 13.101 | 1.00 | 20.78 | C |
| ATOM | 964 | O | THR | A | 311 | 13.271 | 26.953 | 13.972 | 1.00 | 20.12 | O |
| ATOM | 965 | CB | THR | A | 311 | 13.012 | 27.010 | 10.768 | 1.00 | 21.45 | C |
| ATOM | 966 | CG2 | THR | A | 311 | 12.648 | 25.604 | 11.244 | 1.00 | 19.00 | C |
| ATOM | 967 | OG1 | THR | A | 311 | 11.800 | 27.771 | 10.612 | 1.00 | 22.55 | O |
| ATOM | 968 | N | ALA | A | 312 | 12.429 | 28.905 | 13.243 | 1.00 | 20.43 | N |
| ATOM | 969 | CA | ALA | A | 312 | 11.684 | 29.095 | 14.478 | 1.00 | 21.39 | C |
| ATOM | 970 | C | ALA | A | 312 | 10.306 | 28.410 | 14.550 | 1.00 | 20.72 | C |
| ATOM | 971 | O | ALA | A | 312 | 9.931 | 27.917 | 15.617 | 1.00 | 20.75 | O |
| ATOM | 972 | CB | ALA | A | 312 | 11.557 | 30.603 | 14.764 | 1.00 | 19.39 | C |
| ATOM | 973 | N | ARG | A | 313 | 9.572 | 28.341 | 13.433 | 1.00 | 21.51 | N |
| ATOM | 974 | CA | ARG | A | 313 | 8.220 | 27.759 | 13.452 | 1.00 | 23.07 | C |
| ATOM | 975 | C | ARG | A | 313 | 7.752 | 26.671 | 12.480 | 1.00 | 25.81 | C |
| ATOM | 976 | O | ARG | A | 313 | 6.699 | 26.065 | 12.704 | 1.00 | 31.11 | O |
| ATOM | 977 | CB | ARG | A | 313 | 7.183 | 28.871 | 13.360 | 1.00 | 22.88 | C |
| ATOM | 978 | CG | ARG | A | 313 | 7.106 | 29.787 | 14.542 | 1.00 | 22.54 | C |
| ATOM | 979 | CD | ARG | A | 313 | 5.926 | 30.685 | 14.331 | 1.00 | 21.93 | C |
| ATOM | 980 | NE | ARG | A | 313 | 6.123 | 31.470 | 13.133 | 1.00 | 17.29 | N |
| ATOM | 981 | CZ | ARG | A | 313 | 6.930 | 32.516 | 13.076 | 1.00 | 18.99 | C |
| ATOM | 982 | NH1 | ARG | A | 313 | 7.596 | 32.893 | 14.159 | 1.00 | 16.27 | N |
| ATOM | 983 | NH2 | ARG | A | 313 | 7.094 | 33.166 | 11.934 | 1.00 | 19.94 | N |
| ATOM | 984 | N | ILE | A | 314 | 8.469 | 26.421 | 11.403 | 1.00 | 26.71 | N |
| ATOM | 985 | CA | ILE | A | 314 | 8.006 | 25.395 | 10.467 | 1.00 | 29.52 | C |
| ATOM | 986 | C | ILE | A | 314 | 6.759 | 25.838 | 9.697 | 1.00 | 29.85 | C |
| ATOM | 987 | O | ILE | A | 314 | 5.635 | 25.792 | 10.218 | 1.00 | 26.28 | O |
| ATOM | 988 | CB | ILE | A | 314 | 7.639 | 24.054 | 11.169 | 1.00 | 27.99 | C |
| ATOM | 989 | CG1 | ILE | A | 314 | 8.872 | 23.411 | 11.796 | 1.00 | 27.92 | C |
| ATOM | 990 | CG2 | ILE | A | 314 | 7.059 | 23.075 | 10.147 | 1.00 | 29.38 | C |
| ATOM | 991 | CD1 | ILE | A | 314 | 8.557 | 22.107 | 12.501 | 1.00 | 26.06 | C |
| ATOM | 992 | N | TYR | A | 315 | 6.977 | 26.245 | 8.454 | 1.00 | 31.56 | N |
| ATOM | 993 | CA | TYR | A | 315 | 5.910 | 26.680 | 7.576 | 1.00 | 37.08 | C |
| ATOM | 994 | C | TYR | A | 315 | 4.868 | 25.573 | 7.472 | 1.00 | 39.40 | C |
| ATOM | 995 | O | TYR | A | 315 | 3.758 | 25.702 | 7.982 | 1.00 | 41.94 | O |
| ATOM | 996 | CB | TYR | A | 315 | 6.467 | 26.993 | 6.183 | 1.00 | 39.65 | C |
| ATOM | 997 | CG | TYR | A | 315 | 5.423 | 27.453 | 5.194 | 1.00 | 42.46 | C |
| ATOM | 998 | CD1 | TYR | A | 315 | 5.597 | 27.259 | 3.827 | 1.00 | 43.47 | C |
| ATOM | 999 | CD2 | TYR | A | 315 | 4.252 | 28.075 | 5.629 | 1.00 | 44.97 | C |
| ATOM | 1000 | CE1 | TYR | A | 315 | 4.627 | 27.673 | 2.912 | 1.00 | 45.64 | C |
| ATOM | 1001 | CE2 | TYR | A | 315 | 3.280 | 28.493 | 4.727 | 1.00 | 46.56 | C |
| ATOM | 1002 | CZ | TYR | A | 315 | 3.469 | 28.287 | 3.374 | 1.00 | 46.43 | C |
| ATOM | 1003 | OH | TYR | A | 315 | 2.492 | 28.690 | 2.493 | 1.00 | 47.84 | O |
| ATOM | 1004 | N | ASP | A | 316 | 5.229 | 24.480 | 6.818 | 1.00 | 40.75 | N |
| ATOM | 1005 | CA | ASP | A | 316 | 4.301 | 23.371 | 6.671 | 1.00 | 44.52 | C |
| ATOM | 1006 | C | ASP | A | 316 | 2.932 | 23.777 | 6.131 | 1.00 | 45.97 | C |
| ATOM | 1007 | O | ASP | A | 316 | 2.021 | 24.099 | 6.896 | 1.00 | 45.04 | O |
| ATOM | 1008 | CB | ASP | A | 316 | 4.083 | 22.660 | 8.002 | 1.00 | 44.97 | C |
| ATOM | 1009 | CG | ASP | A | 316 | 3.389 | 21.330 | 7.821 | 1.00 | 47.16 | C |
| ATOM | 1010 | OD1 | ASP | A | 316 | 3.024 | 20.680 | 8.826 | 1.00 | 48.70 | O |
| ATOM | 1011 | OD2 | ASP | A | 316 | 3.216 | 20.927 | 6.653 | 1.00 | 46.80 | O |
| ATOM | 1012 | N | ASP | A | 317 | 2.785 | 23.739 | 4.810 | 1.00 | 47.85 | N |
| ATOM | 1013 | CA | ASP | A | 317 | 1.517 | 24.076 | 4.178 | 1.00 | 48.82 | C |
| ATOM | 1014 | C | ASP | A | 317 | 0.666 | 22.807 | 4.135 | 1.00 | 48.88 | C |
| ATOM | 1015 | O | ASP | A | 317 | −0.317 | 22.732 | 3.402 | 1.00 | 48.08 | O |
| ATOM | 1016 | CB | ASP | A | 317 | 1.759 | 24.585 | 2.761 | 1.00 | 49.42 | C |
| ATOM | 1017 | CG | ASP | A | 317 | 1.984 | 23.461 | 1.772 | 1.00 | 49.36 | C |
| ATOM | 1018 | OD1 | ASP | A | 317 | 2.627 | 22.452 | 2.140 | 1.00 | 47.03 | O |
| ATOM | 1019 | OD2 | ASP | A | 317 | 1.522 | 23.600 | 0.625 | 1.00 | 51.14 | O |
| ATOM | 1020 | N | GLN | A | 318 | 1.072 | 21.811 | 4.920 | 1.00 | 49.72 | N |
| ATOM | 1021 | CA | GLN | A | 318 | 0.366 | 20.537 | 4.995 | 1.00 | 51.56 | C |
| ATOM | 1022 | C | GLN | A | 318 | 0.337 | 19.808 | 3.653 | 1.00 | 52.15 | C |
| ATOM | 1023 | O | GLN | A | 318 | −0.159 | 18.684 | 3.560 | 1.00 | 53.90 | O |
| ATOM | 1024 | CB | GLN | A | 318 | −1.064 | 20.765 | 5.492 | 1.00 | 53.86 | C |
| ATOM | 1025 | CG | GLN | A | 318 | −1.142 | 21.340 | 6.899 | 1.00 | 55.42 | C |
| ATOM | 1026 | CD | GLN | A | 318 | −2.545 | 21.786 | 7.256 | 1.00 | 57.27 | C |
| ATOM | 1027 | NE2 | GLN | A | 318 | −3.458 | 21.689 | 6.291 | 1.00 | 58.45 | N |
| ATOM | 1028 | OE1 | GLN | A | 318 | −2.807 | 22.220 | 8.382 | 1.00 | 58.59 | O |
| ATOM | 1029 | N | GLY | A | 319 | 0.881 | 20.448 | 2.620 | 1.00 | 51.88 | N |
| ATOM | 1030 | CA | GLY | A | 319 | 0.901 | 19.849 | 1.297 | 1.00 | 48.99 | C |
| ATOM | 1031 | C | GLY | A | 319 | 2.233 | 19.242 | 0.891 | 1.00 | 47.36 | C |
| ATOM | 1032 | O | GLY | A | 319 | 2.536 | 18.104 | 1.245 | 1.00 | 48.03 | O |
| ATOM | 1033 | N | ARG | A | 320 | 3.027 | 20.011 | 0.149 | 1.00 | 45.19 | N |
| ATOM | 1034 | CA | ARG | A | 320 | 4.330 | 19.573 | −0.342 | 1.00 | 42.20 | C |
| ATOM | 1035 | C | ARG | A | 320 | 5.497 | 20.101 | 0.500 | 1.00 | 39.80 | C |
| ATOM | 1036 | O | ARG | A | 320 | 6.654 | 19.756 | 0.250 | 1.00 | 39.02 | O |
| ATOM | 1037 | CB | ARG | A | 320 | 4.499 | 20.037 | −1.789 | 1.00 | 44.10 | C |
| ATOM | 1038 | CG | ARG | A | 320 | 5.077 | 19.016 | −2.729 | 1.00 | 46.38 | C |
| ATOM | 1039 | CD | ARG | A | 320 | 4.715 | 19.383 | −4.153 | 1.00 | 48.60 | C |
| ATOM | 1040 | NE | ARG | A | 320 | 5.163 | 18.373 | −5.105 | 1.00 | 53.18 | N |
| ATOM | 1041 | CZ | ARG | A | 320 | 6.439 | 18.078 | −5.336 | 1.00 | 55.08 | C |
| ATOM | 1042 | NH1 | ARG | A | 320 | 7.399 | 18.721 | −4.681 | 1.00 | 57.26 | N |
| ATOM | 1043 | NH2 | ARG | A | 320 | 6.755 | 17.141 | −6.221 | 1.00 | 54.44 | N |
| ATOM | 1044 | N | CYS | A | 321 | 5.204 | 20.943 | 1.485 | 1.00 | 35.98 | N |
| ATOM | 1045 | CA | CYS | A | 321 | 6.257 | 21.491 | 2.342 | 1.00 | 33.74 | C |
| ATOM | 1046 | C | CYS | A | 321 | 7.030 | 20.387 | 3.041 | 1.00 | 31.22 | C |
| ATOM | 1047 | O | CYS | A | 321 | 8.262 | 20.411 | 3.081 | 1.00 | 31.55 | O |
| ATOM | 1048 | CB | CYS | A | 321 | 5.675 | 22.441 | 3.392 | 1.00 | 32.82 | C |
| ATOM | 1049 | SG | CYS | A | 321 | 5.173 | 24.053 | 2.748 | 1.00 | 36.76 | S |
| ATOM | 1050 | N | GLN | A | 322 | 6.310 | 19.415 | 3.588 | 1.00 | 29.01 | N |
| ATOM | 1051 | CA | GLN | A | 322 | 6.949 | 18.304 | 4.287 | 1.00 | 29.55 | C |
| ATOM | 1052 | C | GLN | A | 322 | 7.905 | 17.559 | 3.367 | 1.00 | 28.80 | C |
| ATOM | 1053 | O | GLN | A | 322 | 9.016 | 17.192 | 3.759 | 1.00 | 27.56 | O |
| ATOM | 1054 | CB | GLN | A | 322 | 5.890 | 17.345 | 4.830 | 1.00 | 32.20 | C |
| ATOM | 1055 | CG | GLN | A | 322 | 5.070 | 17.932 | 5.973 | 1.00 | 33.57 | C |
| ATOM | 1056 | CD | GLN | A | 322 | 4.273 | 16.874 | 6.694 | 1.00 | 34.78 | C |
| ATOM | 1057 | NE2 | GLN | A | 322 | 3.121 | 17.258 | 7.243 | 1.00 | 35.62 | N |
| ATOM | 1058 | OE1 | GLN | A | 322 | 4.692 | 15.723 | 6.769 | 1.00 | 36.28 | O |
| ATOM | 1059 | N | GLU | A | 323 | 7.467 | 17.327 | 2.138 | 1.00 | 27.83 | N |
| ATOM | 1060 | CA | GLU | A | 323 | 8.306 | 16.642 | 1.178 | 1.00 | 28.38 | C |
| ATOM | 1061 | C | GLU | A | 323 | 9.532 | 17.520 | 0.890 | 1.00 | 27.36 | C |
| ATOM | 1062 | O | GLU | A | 323 | 10.641 | 17.015 | 0.705 | 1.00 | 26.54 | O |
| ATOM | 1063 | CB | GLU | A | 323 | 7.511 | 16.384 | −0.102 | 1.00 | 32.03 | C |
| ATOM | 1064 | CG | GLU | A | 323 | 8.315 | 15.797 | −1.235 | 1.00 | 35.34 | C |
| ATOM | 1065 | CD | GLU | A | 323 | 7.521 | 15.707 | −2.529 | 1.00 | 39.72 | C |
| ATOM | 1066 | OE1 | GLU | A | 323 | 8.146 | 15.415 | −3.577 | 1.00 | 41.00 | O |
| ATOM | 1067 | OE2 | GLU | A | 323 | 6.284 | 15.924 | −2.501 | 1.00 | 39.55 | O |
| ATOM | 1068 | N | GLY | A | 324 | 9.324 | 18.836 | 0.881 | 1.00 | 25.95 | N |
| ATOM | 1069 | CA | GLY | A | 324 | 10.406 | 19.775 | 0.622 | 1.00 | 25.02 | C |
| ATOM | 1070 | C | GLY | A | 324 | 11.521 | 19.700 | 1.646 | 1.00 | 24.88 | C |
| ATOM | 1071 | O | GLY | A | 324 | 12.707 | 19.681 | 1.289 | 1.00 | 25.38 | O |
| ATOM | 1072 | N | LEU | A | 325 | 11.144 | 19.676 | 2.922 | 1.00 | 22.35 | N |
| ATOM | 1073 | CA | LEU | A | 325 | 12.117 | 19.564 | 4.004 | 1.00 | 22.01 | C |
| ATOM | 1074 | C | LEU | A | 325 | 12.836 | 18.216 | 3.850 | 1.00 | 21.77 | C |
| ATOM | 1075 | O | LEU | A | 325 | 14.039 | 18.133 | 4.058 | 1.00 | 21.40 | O |
| ATOM | 1076 | CB | LEU | A | 325 | 11.415 | 19.659 | 5.379 | 1.00 | 22.20 | C |
| ATOM | 1077 | CG | LEU | A | 325 | 10.572 | 20.925 | 5.664 | 1.00 | 22.58 | C |
| ATOM | 1078 | CD1 | LEU | A | 325 | 10.078 | 20.933 | 7.097 | 1.00 | 21.89 | C |
| ATOM | 1079 | CD2 | LEU | A | 325 | 11.406 | 22.164 | 5.412 | 1.00 | 22.30 | C |
| ATOM | 1080 | N | ARG | A | 326 | 12.121 | 17.159 | 3.464 | 1.00 | 20.96 | N |
| ATOM | 1081 | CA | ARG | A | 326 | 12.802 | 15.881 | 3.293 | 1.00 | 22.65 | C |
| ATOM | 1082 | C | ARG | A | 326 | 13.812 | 15.952 | 2.161 | 1.00 | 23.02 | C |
| ATOM | 1083 | O | ARG | A | 326 | 14.857 | 15.299 | 2.218 | 1.00 | 21.38 | O |
| ATOM | 1084 | CB | ARG | A | 326 | 11.807 | 14.751 | 3.030 | 1.00 | 22.55 | C |
| ATOM | 1085 | CG | ARG | A | 326 | 11.013 | 14.359 | 4.272 | 1.00 | 25.34 | C |
| ATOM | 1086 | CD | ARG | A | 326 | 10.287 | 13.059 | 4.057 | 1.00 | 25.61 | C |
| ATOM | 1087 | NE | ARG | A | 326 | 9.149 | 13.224 | 3.158 | 1.00 | 27.36 | N |
| ATOM | 1088 | CZ | ARG | A | 326 | 7.951 | 13.622 | 3.565 | 1.00 | 29.26 | C |
| ATOM | 1089 | NH1 | ARG | A | 326 | 7.751 | 13.891 | 4.847 | 1.00 | 30.59 | N |
| ATOM | 1090 | NH2 | ARG | A | 326 | 6.955 | 13.730 | 2.699 | 1.00 | 31.98 | N |
| ATOM | 1091 | N | THR | A | 327 | 13.501 | 16.759 | 1.146 | 1.00 | 23.09 | N |
| ATOM | 1092 | CA | THR | A | 327 | 14.371 | 16.937 | −0.025 | 1.00 | 22.71 | C |
| ATOM | 1093 | C | THR | A | 327 | 15.622 | 17.743 | 0.322 | 1.00 | 21.89 | C |
| ATOM | 1094 | O | THR | A | 327 | 16.715 | 17.458 | −0.162 | 1.00 | 21.56 | O |
| ATOM | 1095 | CB | THR | A | 327 | 13.613 | 17.658 | −1.172 | 1.00 | 24.13 | C |
| ATOM | 1096 | CG2 | THR | A | 327 | 14.534 | 17.905 | −2.358 | 1.00 | 23.18 | C |
| ATOM | 1097 | OG1 | THR | A | 327 | 12.510 | 16.843 | −1.600 | 1.00 | 27.01 | O |
| ATOM | 1098 | N | LEU | A | 328 | 15.456 | 18.757 | 1.159 | 1.00 | 21.54 | N |
| ATOM | 1099 | CA | LEU | A | 328 | 16.587 | 19.565 | 1.569 | 1.00 | 22.94 | C |
| ATOM | 1100 | C | LEU | A | 328 | 17.552 | 18.705 | 2.394 | 1.00 | 22.64 | C |
| ATOM | 1101 | O | LEU | A | 328 | 18.768 | 18.767 | 2.212 | 1.00 | 22.69 | O |
| ATOM | 1102 | CB | LEU | A | 328 | 16.110 | 20.746 | 2.414 | 1.00 | 23.87 | C |
| ATOM | 1103 | CG | LEU | A | 328 | 16.513 | 22.123 | 1.937 | 1.00 | 24.68 | C |
| ATOM | 1104 | CD1 | LEU | A | 328 | 16.099 | 23.151 | 2.982 | 1.00 | 23.78 | C |
| ATOM | 1105 | CD2 | LEU | A | 328 | 18.016 | 22.176 | 1.687 | 1.00 | 25.93 | C |
| ATOM | 1106 | N | ALA | A | 329 | 17.007 | 17.911 | 3.308 | 1.00 | 21.42 | N |
| ATOM | 1107 | CA | ALA | A | 329 | 17.850 | 17.055 | 4.128 | 1.00 | 22.64 | C |
| ATOM | 1108 | C | ALA | A | 329 | 18.523 | 16.021 | 3.245 | 1.00 | 21.91 | C |
| ATOM | 1109 | O | ALA | A | 329 | 19.698 | 15.699 | 3.428 | 1.00 | 22.46 | O |
| ATOM | 1110 | CB | ALA | A | 329 | 17.033 | 16.363 | 5.196 | 1.00 | 21.28 | C |
| ATOM | 1111 | N | GLU | A | 330 | 17.777 | 15.503 | 2.280 | 1.00 | 21.55 | N |
| ATOM | 1112 | CA | GLU | A | 330 | 18.336 | 14.502 | 1.392 | 1.00 | 21.81 | C |
| ATOM | 1113 | C | GLU | A | 330 | 19.532 | 15.106 | 0.689 | 1.00 | 20.66 | C |
| ATOM | 1114 | O | GLU | A | 330 | 20.547 | 14.439 | 0.463 | 1.00 | 19.46 | O |
| ATOM | 1115 | CB | GLU | A | 330 | 17.303 | 14.071 | 0.356 | 1.00 | 25.02 | C |
| ATOM | 1116 | CG | GLU | A | 330 | 17.724 | 12.863 | −0.460 | 1.00 | 29.62 | C |
| ATOM | 1117 | CD | GLU | A | 330 | 16.528 | 12.160 | −1.090 | 1.00 | 32.07 | C |
| ATOM | 1118 | OE1 | GLU | A | 330 | 15.494 | 12.029 | −0.398 | 1.00 | 30.78 | O |
| ATOM | 1119 | OE2 | GLU | A | 330 | 16.630 | 11.739 | −2.262 | 1.00 | 32.02 | O |
| ATOM | 1120 | N | ALA | A | 331 | 19.406 | 16.384 | 0.359 | 1.00 | 19.29 | N |
| ATOM | 1121 | CA | ALA | A | 331 | 20.464 | 17.102 | −0.335 | 1.00 | 20.58 | C |
| ATOM | 1122 | C | ALA | A | 331 | 21.681 | 17.371 | 0.545 | 1.00 | 19.80 | C |
| ATOM | 1123 | O | ALA | A | 331 | 22.688 | 17.871 | 0.061 | 1.00 | 20.62 | O |
| ATOM | 1124 | CB | ALA | A | 331 | 19.921 | 18.405 | −0.900 | 1.00 | 15.81 | C |
| ATOM | 1125 | N | GLY | A | 332 | 21.593 | 17.035 | 1.829 | 1.00 | 22.50 | N |
| ATOM | 1126 | CA | GLY | A | 332 | 22.730 | 17.262 | 2.710 | 1.00 | 23.09 | C |
| ATOM | 1127 | C | GLY | A | 332 | 22.598 | 18.430 | 3.675 | 1.00 | 23.40 | C |
| ATOM | 1128 | O | GLY | A | 332 | 23.380 | 18.551 | 4.616 | 1.00 | 24.38 | O |
| ATOM | 1129 | N | ALA | A | 333 | 21.623 | 19.304 | 3.466 | 1.00 | 23.30 | N |
| ATOM | 1130 | CA | ALA | A | 333 | 21.463 | 20.424 | 4.383 | 1.00 | 21.65 | C |
| ATOM | 1131 | C | ALA | A | 333 | 21.009 | 19.961 | 5.768 | 1.00 | 23.96 | C |
| ATOM | 1132 | O | ALA | A | 333 | 20.253 | 18.995 | 5.905 | 1.00 | 24.50 | O |
| ATOM | 1133 | CB | ALA | A | 333 | 20.469 | 21.412 | 3.831 | 1.00 | 22.51 | C |
| ATOM | 1134 | N | LYS | A | 334 | 21.492 | 20.639 | 6.803 | 1.00 | 24.11 | N |
| ATOM | 1135 | CA | LYS | A | 334 | 21.058 | 20.316 | 8.146 | 1.00 | 23.25 | C |
| ATOM | 1136 | C | LYS | A | 334 | 19.892 | 21.245 | 8.447 | 1.00 | 21.92 | C |
| ATOM | 1137 | O | LYS | A | 334 | 20.016 | 22.470 | 8.357 | 1.00 | 19.18 | O |
| ATOM | 1138 | CB | LYS | A | 334 | 22.161 | 20.544 | 9.183 | 1.00 | 24.05 | C |
| ATOM | 1139 | CG | LYS | A | 334 | 21.688 | 20.245 | 10.619 | 1.00 | 22.88 | C |
| ATOM | 1140 | CD | LYS | A | 334 | 22.770 | 20.513 | 11.657 | 1.00 | 23.15 | C |
| ATOM | 1141 | CE | LYS | A | 334 | 22.334 | 20.071 | 13.053 | 1.00 | 20.79 | C |
| ATOM | 1142 | NZ | LYS | A | 334 | 23.389 | 20.313 | 14.073 | 1.00 | 18.92 | N |
| ATOM | 1143 | N | ILE | A | 335 | 18.751 | 20.652 | 8.772 | 1.00 | 21.84 | N |
| ATOM | 1144 | CA | ILE | A | 335 | 17.563 | 21.421 | 9.097 | 1.00 | 21.81 | C |
| ATOM | 1145 | C | ILE | A | 335 | 17.207 | 21.127 | 10.541 | 1.00 | 22.22 | C |
| ATOM | 1146 | O | ILE | A | 335 | 17.005 | 19.976 | 10.926 | 1.00 | 22.43 | O |
| ATOM | 1147 | CB | ILE | A | 335 | 16.372 | 21.032 | 8.218 | 1.00 | 22.28 | C |
| ATOM | 1148 | CG1 | ILE | A | 335 | 16.769 | 21.118 | 6.740 | 1.00 | 22.93 | C |
| ATOM | 1149 | CG2 | ILE | A | 335 | 15.194 | 21.964 | 8.520 | 1.00 | 21.98 | C |
| ATOM | 1150 | CD1 | ILE | A | 335 | 15.776 | 20.416 | 5.790 | 1.00 | 23.67 | C |
| ATOM | 1151 | N | SER | A | 336 | 17.123 | 22.178 | 11.340 | 1.00 | 21.34 | N |
| ATOM | 1152 | CA | SER | A | 336 | 16.821 | 22.010 | 12.742 | 1.00 | 22.28 | C |
| ATOM | 1153 | C | SER | A | 336 | 15.917 | 23.127 | 13.245 | 1.00 | 20.97 | C |
| ATOM | 1154 | O | SER | A | 336 | 15.742 | 24.145 | 12.577 | 1.00 | 23.06 | O |
| ATOM | 1155 | CB | SER | A | 336 | 18.137 | 21.979 | 13.522 | 1.00 | 21.14 | C |
| ATOM | 1156 | OG | SER | A | 336 | 17.916 | 22.239 | 14.889 | 1.00 | 31.77 | O |
| ATOM | 1157 | N | ILE | A | 337 | 15.343 | 22.921 | 14.423 | 1.00 | 16.98 | N |
| ATOM | 1158 | CA | ILE | A | 337 | 14.463 | 23.896 | 15.048 | 1.00 | 14.55 | C |
| ATOM | 1159 | C | ILE | A | 337 | 15.287 | 24.732 | 16.029 | 1.00 | 14.77 | C |
| ATOM | 1160 | O | ILE | A | 337 | 16.161 | 24.206 | 16.739 | 1.00 | 12.16 | O |
| ATOM | 1161 | CB | ILE | A | 337 | 13.354 | 23.197 | 15.864 | 1.00 | 15.40 | C |
| ATOM | 1162 | CG1 | ILE | A | 337 | 12.577 | 22.214 | 14.974 | 1.00 | 13.20 | C |
| ATOM | 1163 | CG2 | ILE | A | 337 | 12.482 | 24.242 | 16.535 | 1.00 | 12.45 | C |
| ATOM | 1164 | CD1 | ILE | A | 337 | 11.729 | 22.864 | 13.897 | 1.00 | 14.63 | C |
| ATOM | 1165 | N | MET | A | 338 | 15.001 | 26.030 | 16.061 | 1.00 | 13.85 | N |
| ATOM | 1166 | CA | MET | A | 338 | 15.680 | 26.968 | 16.949 | 1.00 | 13.80 | C |
| ATOM | 1167 | C | MET | A | 338 | 15.275 | 26.722 | 18.417 | 1.00 | 16.14 | C |
| ATOM | 1168 | O | MET | A | 338 | 14.110 | 26.467 | 18.713 | 1.00 | 16.12 | O |
| ATOM | 1169 | CB | MET | A | 338 | 15.333 | 28.423 | 16.553 | 1.00 | 13.31 | C |
| ATOM | 1170 | CG | MET | A | 338 | 15.933 | 28.946 | 15.201 | 1.00 | 11.43 | C |
| ATOM | 1171 | SD | MET | A | 338 | 15.463 | 30.673 | 14.801 | 1.00 | 5.66 | S |
| ATOM | 1172 | CE | MET | A | 338 | 16.554 | 31.583 | 16.040 | 1.00 | 15.50 | C |
| ATOM | 1173 | N | THR | A | 339 | 16.249 | 26.779 | 19.319 | 1.00 | 18.09 | N |
| ATOM | 1174 | CA | THR | A | 339 | 16.020 | 26.590 | 20.754 | 1.00 | 17.70 | C |
| ATOM | 1175 | C | THR | A | 339 | 16.536 | 27.832 | 21.447 | 1.00 | 18.83 | C |
| ATOM | 1176 | O | THR | A | 339 | 16.911 | 28.806 | 20.789 | 1.00 | 19.25 | O |
| ATOM | 1177 | CB | THR | A | 339 | 16.813 | 25.396 | 21.319 | 1.00 | 18.11 | C |
| ATOM | 1178 | CG2 | THR | A | 339 | 16.256 | 24.093 | 20.801 | 1.00 | 20.92 | C |
| ATOM | 1179 | OG1 | THR | A | 339 | 18.185 | 25.512 | 20.924 | 1.00 | 20.52 | O |
| ATOM | 1180 | N | TYR | A | 340 | 16.571 | 27.802 | 22.773 | 1.00 | 18.47 | N |
| ATOM | 1181 | CA | TYR | A | 340 | 17.058 | 28.947 | 23.518 | 1.00 | 18.07 | C |
| ATOM | 1182 | C | TYR | A | 340 | 18.392 | 29.462 | 22.954 | 1.00 | 18.46 | C |
| ATOM | 1183 | O | TYR | A | 340 | 18.582 | 30.667 | 22.806 | 1.00 | 19.45 | O |
| ATOM | 1184 | CB | TYR | A | 340 | 17.250 | 28.583 | 24.991 | 1.00 | 19.10 | C |
| ATOM | 1185 | CG | TYR | A | 340 | 17.883 | 29.713 | 25.763 | 1.00 | 20.47 | C |
| ATOM | 1186 | CD1 | TYR | A | 340 | 17.129 | 30.806 | 26.176 | 1.00 | 19.27 | C |
| ATOM | 1187 | CD2 | TYR | A | 340 | 19.251 | 29.716 | 26.024 | 1.00 | 18.37 | C |
| ATOM | 1188 | CE1 | TYR | A | 340 | 17.722 | 31.874 | 26.830 | 1.00 | 22.56 | C |
| ATOM | 1189 | CE2 | TYR | A | 340 | 19.852 | 30.778 | 26.673 | 1.00 | 21.01 | C |
| ATOM | 1190 | CZ | TYR | A | 340 | 19.086 | 31.852 | 27.076 | 1.00 | 21.14 | C |
| ATOM | 1191 | OH | TYR | A | 340 | 19.671 | 32.907 | 27.731 | 1.00 | 24.59 | O |
| ATOM | 1192 | N | SER | A | 341 | 19.312 | 28.551 | 22.641 | 1.00 | 17.85 | N |
| ATOM | 1193 | CA | SER | A | 341 | 20.622 | 28.943 | 22.108 | 1.00 | 18.03 | C |
| ATOM | 1194 | C | SER | A | 341 | 20.606 | 29.706 | 20.779 | 1.00 | 17.13 | C |
| ATOM | 1195 | O | SER | A | 341 | 21.291 | 30.720 | 20.624 | 1.00 | 17.29 | O |
| ATOM | 1196 | CB | SER | A | 341 | 21.519 | 27.717 | 21.967 | 1.00 | 17.63 | C |
| ATOM | 1197 | OG | SER | A | 341 | 21.829 | 27.208 | 23.240 | 1.00 | 23.08 | O |
| ATOM | 1198 | N | GLU | A | 342 | 19.847 | 29.214 | 19.811 | 1.00 | 17.19 | N |
| ATOM | 1199 | CA | GLU | A | 342 | 19.784 | 29.894 | 18.528 | 1.00 | 15.18 | C |
| ATOM | 1200 | C | GLU | A | 342 | 19.116 | 31.261 | 18.666 | 1.00 | 15.80 | C |
| ATOM | 1201 | O | GLU | A | 342 | 19.564 | 32.231 | 18.067 | 1.00 | 15.05 | O |
| ATOM | 1202 | CB | GLU | A | 342 | 19.041 | 29.034 | 17.507 | 1.00 | 16.89 | C |
| ATOM | 1203 | CG | GLU | A | 342 | 19.832 | 27.811 | 17.027 | 1.00 | 16.59 | C |
| ATOM | 1204 | CD | GLU | A | 342 | 20.001 | 26.765 | 18.110 | 1.00 | 18.25 | C |
| ATOM | 1205 | OE1 | GLU | A | 342 | 18.991 | 26.411 | 18.763 | 1.00 | 16.87 | O |
| ATOM | 1206 | OE2 | GLU | A | 342 | 21.139 | 26.297 | 18.310 | 1.00 | 17.58 | O |
| ATOM | 1207 | N | PHE | A | 343 | 18.055 | 31.344 | 19.467 | 1.00 | 16.03 | N |
| ATOM | 1208 | CA | PHE | A | 343 | 17.358 | 32.610 | 19.664 | 1.00 | 16.11 | C |
| ATOM | 1209 | C | PHE | A | 343 | 18.260 | 33.668 | 20.325 | 1.00 | 16.62 | C |
| ATOM | 1210 | O | PHE | A | 343 | 18.295 | 34.831 | 19.896 | 1.00 | 17.04 | O |
| ATOM | 1211 | CB | PHE | A | 343 | 16.109 | 32.391 | 20.513 | 1.00 | 17.75 | C |
| ATOM | 1212 | CG | PHE | A | 343 | 15.017 | 31.611 | 19.817 | 1.00 | 18.83 | C |
| ATOM | 1213 | CD1 | PHE | A | 343 | 14.503 | 30.456 | 20.390 | 1.00 | 18.07 | C |
| ATOM | 1214 | CD2 | PHE | A | 343 | 14.440 | 32.091 | 18.640 | 1.00 | 20.43 | C |
| ATOM | 1215 | CE1 | PHE | A | 343 | 13.417 | 29.788 | 19.815 | 1.00 | 19.37 | C |
| ATOM | 1216 | CE2 | PHE | A | 343 | 13.357 | 31.434 | 18.056 | 1.00 | 20.69 | C |
| ATOM | 1217 | CZ | PHE | A | 343 | 12.842 | 30.283 | 18.644 | 1.00 | 19.47 | C |
| ATOM | 1218 | N | LYS | A | 344 | 18.993 | 33.269 | 21.360 | 1.00 | 16.02 | N |
| ATOM | 1219 | CA | LYS | A | 344 | 19.888 | 34.191 | 22.061 | 1.00 | 17.65 | C |
| ATOM | 1220 | C | LYS | A | 344 | 20.993 | 34.656 | 21.105 | 1.00 | 17.67 | C |
| ATOM | 1221 | O | LYS | A | 344 | 21.331 | 35.840 | 21.053 | 1.00 | 16.64 | O |
| ATOM | 1222 | CB | LYS | A | 344 | 20.499 | 33.495 | 23.290 | 1.00 | 18.19 | C |
| ATOM | 1223 | CG | LYS | A | 344 | 21.534 | 34.331 | 24.059 | 1.00 | 21.58 | C |
| ATOM | 1224 | CD | LYS | A | 344 | 22.142 | 33.519 | 25.201 | 1.00 | 26.60 | C |
| ATOM | 1225 | CE | LYS | A | 344 | 22.990 | 34.372 | 26.139 | 1.00 | 31.42 | C |
| ATOM | 1226 | NZ | LYS | A | 344 | 24.350 | 34.688 | 25.583 | 1.00 | 35.26 | N |
| ATOM | 1227 | N | HIS | A | 345 | 21.549 | 33.711 | 20.355 | 1.00 | 19.16 | N |
| ATOM | 1228 | CA | HIS | A | 345 | 22.593 | 34.025 | 19.390 | 1.00 | 20.53 | C |
| ATOM | 1229 | C | HIS | A | 345 | 22.109 | 35.122 | 18.430 | 1.00 | 21.40 | C |
| ATOM | 1230 | O | HIS | A | 345 | 22.745 | 36.170 | 18.287 | 1.00 | 19.93 | O |
| ATOM | 1231 | CB | HIS | A | 345 | 22.964 | 32.776 | 18.580 | 1.00 | 21.01 | C |
| ATOM | 1232 | CG | HIS | A | 345 | 24.038 | 33.024 | 17.570 | 1.00 | 23.09 | C |
| ATOM | 1233 | CD2 | HIS | A | 345 | 23.972 | 33.248 | 16.235 | 1.00 | 21.54 | C |
| ATOM | 1234 | ND1 | HIS | A | 345 | 25.360 | 33.180 | 17.922 | 1.00 | 22.09 | N |
| ATOM | 1235 | CE1 | HIS | A | 345 | 26.063 | 33.497 | 16.849 | 1.00 | 24.59 | C |
| ATOM | 1236 | NE2 | HIS | A | 345 | 25.244 | 33.545 | 15.812 | 1.00 | 25.04 | N |
| ATOM | 1237 | N | CYS | A | 346 | 20.980 | 34.874 | 17.774 | 1.00 | 20.75 | N |
| ATOM | 1238 | CA | CYS | A | 346 | 20.426 | 35.853 | 16.848 | 1.00 | 19.07 | C |
| ATOM | 1239 | C | CYS | A | 346 | 20.186 | 37.189 | 17.523 | 1.00 | 18.29 | C |
| ATOM | 1240 | O | CYS | A | 346 | 20.461 | 38.231 | 16.948 | 1.00 | 17.67 | O |
| ATOM | 1241 | CB | CYS | A | 346 | 19.116 | 35.347 | 16.252 | 1.00 | 20.89 | C |
| ATOM | 1242 | SG | CYS | A | 346 | 19.327 | 33.924 | 15.168 | 1.00 | 31.86 | S |
| ATOM | 1243 | N | TRP | A | 347 | 19.656 | 37.163 | 18.744 | 1.00 | 20.14 | N |
| ATOM | 1244 | CA | TRP | A | 347 | 19.398 | 38.408 | 19.461 | 1.00 | 21.67 | C |
| ATOM | 1245 | C | TRP | A | 347 | 20.689 | 39.194 | 19.649 | 1.00 | 22.70 | C |
| ATOM | 1246 | O | TRP | A | 347 | 20.741 | 40.400 | 19.382 | 1.00 | 20.87 | O |
| ATOM | 1247 | CB | TRP | A | 347 | 18.745 | 38.119 | 20.814 | 1.00 | 21.74 | C |
| ATOM | 1248 | CG | TRP | A | 347 | 18.602 | 39.317 | 21.722 | 1.00 | 23.25 | C |
| ATOM | 1249 | CD1 | TRP | A | 347 | 19.536 | 39.798 | 22.610 | 1.00 | 23.22 | C |
| ATOM | 1250 | CD2 | TRP | A | 347 | 17.463 | 40.180 | 21.839 | 1.00 | 23.59 | C |
| ATOM | 1251 | CE2 | TRP | A | 347 | 17.776 | 41.156 | 22.817 | 1.00 | 25.86 | C |
| ATOM | 1252 | CE3 | TRP | A | 347 | 16.207 | 40.224 | 21.217 | 1.00 | 24.42 | C |
| ATOM | 1253 | NE1 | TRP | A | 347 | 19.045 | 40.900 | 23.268 | 1.00 | 23.38 | N |
| ATOM | 1254 | CZ2 | TRP | A | 347 | 16.875 | 42.165 | 23.185 | 1.00 | 25.25 | C |
| ATOM | 1255 | CZ3 | TRP | A | 347 | 15.309 | 41.231 | 21.584 | 1.00 | 25.21 | C |
| ATOM | 1256 | CH2 | TRP | A | 347 | 15.651 | 42.185 | 22.560 | 1.00 | 24.09 | C |
| ATOM | 1257 | N | ASP | A | 348 | 21.739 | 38.503 | 20.087 | 1.00 | 23.24 | N |
| ATOM | 1258 | CA | ASP | A | 348 | 23.024 | 39.152 | 20.327 | 1.00 | 22.40 | C |
| ATOM | 1259 | C | ASP | A | 348 | 23.762 | 39.506 | 19.058 | 1.00 | 23.89 | C |
| ATOM | 1260 | O | ASP | A | 348 | 24.640 | 40.362 | 19.071 | 1.00 | 25.44 | O |
| ATOM | 1261 | CB | ASP | A | 348 | 23.940 | 38.260 | 21.166 | 1.00 | 22.44 | C |
| ATOM | 1262 | CG | ASP | A | 348 | 23.342 | 37.908 | 22.506 | 1.00 | 21.84 | C |
| ATOM | 1263 | OD1 | ASP | A | 348 | 22.419 | 38.616 | 22.940 | 1.00 | 21.81 | O |
| ATOM | 1264 | OD2 | ASP | A | 348 | 23.803 | 36.931 | 23.124 | 1.00 | 19.91 | O |
| ATOM | 1265 | N | THR | A | 349 | 23.413 | 38.863 | 17.952 | 1.00 | 24.17 | N |
| ATOM | 1266 | CA | THR | A | 349 | 24.142 | 39.137 | 16.731 | 1.00 | 23.57 | C |
| ATOM | 1267 | C | THR | A | 349 | 23.441 | 39.977 | 15.692 | 1.00 | 24.47 | C |
| ATOM | 1268 | O | THR | A | 349 | 24.092 | 40.755 | 14.991 | 1.00 | 23.37 | O |
| ATOM | 1269 | CB | THR | A | 349 | 24.590 | 37.826 | 16.071 | 1.00 | 24.08 | C |
| ATOM | 1270 | CG2 | THR | A | 349 | 25.523 | 38.118 | 14.910 | 1.00 | 24.14 | C |
| ATOM | 1271 | OG1 | THR | A | 349 | 25.278 | 37.020 | 17.038 | 1.00 | 23.82 | O |
| ATOM | 1272 | N | PHE | A | 350 | 22.123 | 39.833 | 15.602 | 1.00 | 22.77 | N |
| ATOM | 1273 | CA | PHE | A | 350 | 21.349 | 40.550 | 14.596 | 1.00 | 22.90 | C |
| ATOM | 1274 | C | PHE | A | 350 | 20.319 | 41.559 | 15.086 | 1.00 | 25.98 | C |
| ATOM | 1275 | O | PHE | A | 350 | 19.586 | 42.134 | 14.270 | 1.00 | 26.06 | O |
| ATOM | 1276 | CB | PHE | A | 350 | 20.628 | 39.550 | 13.694 | 1.00 | 22.65 | C |
| ATOM | 1277 | CG | PHE | A | 350 | 21.548 | 38.608 | 12.978 | 1.00 | 20.55 | C |
| ATOM | 1278 | CD1 | PHE | A | 350 | 21.762 | 37.324 | 13.459 | 1.00 | 19.32 | C |
| ATOM | 1279 | CD2 | PHE | A | 350 | 22.231 | 39.022 | 11.849 | 1.00 | 20.18 | C |
| ATOM | 1280 | CE1 | PHE | A | 350 | 22.655 | 36.463 | 12.818 | 1.00 | 21.40 | C |
| ATOM | 1281 | CE2 | PHE | A | 350 | 23.127 | 38.175 | 11.201 | 1.00 | 21.57 | C |
| ATOM | 1282 | CZ | PHE | A | 350 | 23.337 | 36.891 | 11.689 | 1.00 | 20.10 | C |
| ATOM | 1283 | N | VAL | A | 351 | 20.237 | 41.776 | 16.398 | 1.00 | 22.94 | N |
| ATOM | 1284 | CA | VAL | A | 351 | 19.263 | 42.726 | 16.922 | 1.00 | 23.60 | C |
| ATOM | 1285 | C | VAL | A | 351 | 19.932 | 43.934 | 17.577 | 1.00 | 24.43 | C |
| ATOM | 1286 | O | VAL | A | 351 | 20.998 | 43.820 | 18.178 | 1.00 | 22.97 | O |
| ATOM | 1287 | CB | VAL | A | 351 | 18.309 | 42.055 | 17.947 | 1.00 | 25.23 | C |
| ATOM | 1288 | CG1 | VAL | A | 351 | 17.235 | 43.053 | 18.413 | 1.00 | 22.26 | C |
| ATOM | 1289 | CG2 | VAL | A | 351 | 17.670 | 40.821 | 17.328 | 1.00 | 24.65 | C |
| ATOM | 1290 | N | ASP | A | 352 | 19.301 | 45.097 | 17.428 | 1.00 | 24.62 | N |
| ATOM | 1291 | CA | ASP | A | 352 | 19.798 | 46.343 | 18.010 | 1.00 | 23.02 | C |
| ATOM | 1292 | C | ASP | A | 352 | 19.280 | 46.349 | 19.438 | 1.00 | 20.71 | C |
| ATOM | 1293 | O | ASP | A | 352 | 18.441 | 47.167 | 19.795 | 1.00 | 19.73 | O |
| ATOM | 1294 | CB | ASP | A | 352 | 19.235 | 47.537 | 17.235 | 1.00 | 26.24 | C |
| ATOM | 1295 | CG | ASP | A | 352 | 19.873 | 48.869 | 17.633 | 1.00 | 29.83 | C |
| ATOM | 1296 | OD1 | ASP | A | 352 | 19.783 | 49.820 | 16.830 | 1.00 | 30.30 | O |
| ATOM | 1297 | OD2 | ASP | A | 352 | 20.450 | 48.980 | 18.740 | 1.00 | 35.52 | O |
| ATOM | 1298 | N | HIS | A | 353 | 19.773 | 45.406 | 20.241 | 1.00 | 21.31 | N |
| ATOM | 1299 | CA | HIS | A | 353 | 19.356 | 45.267 | 21.638 | 1.00 | 19.09 | C |
| ATOM | 1300 | C | HIS | A | 353 | 19.785 | 46.447 | 22.501 | 1.00 | 18.18 | C |
| ATOM | 1301 | O | HIS | A | 353 | 19.336 | 46.593 | 23.637 | 1.00 | 21.92 | O |
| ATOM | 1302 | CB | HIS | A | 353 | 19.909 | 43.963 | 22.231 | 1.00 | 16.23 | C |
| ATOM | 1303 | CG | HIS | A | 353 | 21.388 | 43.815 | 22.073 | 1.00 | 16.31 | C |
| ATOM | 1304 | CD2 | HIS | A | 353 | 22.413 | 44.523 | 22.597 | 1.00 | 15.39 | C |
| ATOM | 1305 | ND1 | HIS | A | 353 | 21.957 | 42.898 | 21.214 | 1.00 | 18.73 | N |
| ATOM | 1306 | CE1 | HIS | A | 353 | 23.268 | 43.054 | 21.207 | 1.00 | 17.98 | C |
| ATOM | 1307 | NE2 | HIS | A | 353 | 23.570 | 44.035 | 22.037 | 1.00 | 21.16 | N |
| ATOM | 1308 | N | GLN | A | 354 | 20.666 | 47.282 | 21.973 | 1.00 | 17.36 | N |
| ATOM | 1309 | CA | GLN | A | 354 | 21.113 | 48.451 | 22.709 | 1.00 | 16.76 | C |
| ATOM | 1310 | C | GLN | A | 354 | 21.652 | 48.081 | 24.089 | 1.00 | 18.22 | C |
| ATOM | 1311 | O | GLN | A | 354 | 21.548 | 48.862 | 25.030 | 1.00 | 16.66 | O |
| ATOM | 1312 | CB | GLN | A | 354 | 19.947 | 49.444 | 22.852 | 1.00 | 16.49 | C |
| ATOM | 1313 | CG | GLN | A | 354 | 19.423 | 49.984 | 21.532 | 1.00 | 15.77 | C |
| ATOM | 1314 | CD | GLN | A | 354 | 18.366 | 51.049 | 21.740 | 1.00 | 18.85 | C |
| ATOM | 1315 | NE2 | GLN | A | 354 | 17.295 | 50.985 | 20.964 | 1.00 | 19.83 | N |
| ATOM | 1316 | OE1 | GLN | A | 354 | 18.519 | 51.932 | 22.590 | 1.00 | 22.72 | O |
| ATOM | 1317 | N | GLY | A | 355 | 22.226 | 46.887 | 24.197 | 1.00 | 20.52 | N |
| ATOM | 1318 | CA | GLY | A | 355 | 22.808 | 46.448 | 25.452 | 1.00 | 20.58 | C |
| ATOM | 1319 | C | GLY | A | 355 | 21.938 | 45.535 | 26.292 | 1.00 | 22.88 | C |
| ATOM | 1320 | O | GLY | A | 355 | 22.426 | 44.911 | 27.239 | 1.00 | 23.99 | O |
| ATOM | 1321 | N | CYS | A | 356 | 20.653 | 45.452 | 25.963 | 1.00 | 22.15 | N |
| ATOM | 1322 | CA | CYS | A | 356 | 19.754 | 44.605 | 26.730 | 1.00 | 24.01 | C |
| ATOM | 1323 | C | CYS | A | 356 | 19.970 | 43.136 | 26.444 | 1.00 | 22.68 | C |
| ATOM | 1324 | O | CYS | A | 356 | 19.933 | 42.699 | 25.299 | 1.00 | 21.46 | O |
| ATOM | 1325 | CB | CYS | A | 356 | 18.288 | 44.954 | 26.459 | 1.00 | 25.43 | C |
| ATOM | 1326 | SG | CYS | A | 356 | 17.784 | 46.587 | 27.068 | 1.00 | 30.20 | S |
| ATOM | 1327 | N | PRO | A | 357 | 20.213 | 42.353 | 27.497 | 1.00 | 23.13 | N |
| ATOM | 1328 | CA | PRO | A | 357 | 20.429 | 40.916 | 27.324 | 1.00 | 23.37 | C |
| ATOM | 1329 | C | PRO | A | 357 | 19.143 | 40.275 | 26.812 | 1.00 | 24.11 | C |
| ATOM | 1330 | O | PRO | A | 357 | 18.063 | 40.869 | 26.894 | 1.00 | 23.68 | O |
| ATOM | 1331 | CB | PRO | A | 357 | 20.807 | 40.446 | 28.731 | 1.00 | 24.59 | C |
| ATOM | 1332 | CG | PRO | A | 357 | 21.436 | 41.689 | 29.344 | 1.00 | 24.38 | C |
| ATOM | 1333 | CD | PRO | A | 357 | 20.496 | 42.774 | 28.879 | 1.00 | 23.99 | C |
| ATOM | 1334 | N | PHE | A | 358 | 19.272 | 39.067 | 26.274 | 1.00 | 23.76 | N |
| ATOM | 1335 | CA | PHE | A | 358 | 18.139 | 38.345 | 25.729 | 1.00 | 25.23 | C |
| ATOM | 1336 | C | PHE | A | 358 | 17.201 | 37.858 | 26.825 | 1.00 | 26.06 | C |
| ATOM | 1337 | O | PHE | A | 358 | 17.635 | 37.241 | 27.794 | 1.00 | 25.97 | O |
| ATOM | 1338 | CB | PHE | A | 358 | 18.628 | 37.162 | 24.907 | 1.00 | 24.52 | C |
| ATOM | 1339 | CG | PHE | A | 358 | 17.526 | 36.297 | 24.405 | 1.00 | 26.89 | C |
| ATOM | 1340 | CD1 | PHE | A | 358 | 16.553 | 36.818 | 23.558 | 1.00 | 24.92 | C |
| ATOM | 1341 | CD2 | PHE | A | 358 | 17.436 | 34.963 | 24.793 | 1.00 | 24.77 | C |
| ATOM | 1342 | CE1 | PHE | A | 358 | 15.514 | 36.029 | 23.110 | 1.00 | 22.97 | C |
| ATOM | 1343 | CE2 | PHE | A | 358 | 16.396 | 34.168 | 24.344 | 1.00 | 22.17 | C |
| ATOM | 1344 | CZ | PHE | A | 358 | 15.434 | 34.700 | 23.503 | 1.00 | 20.87 | C |
| ATOM | 1345 | N | GLN | A | 359 | 15.915 | 38.140 | 26.662 | 1.00 | 27.02 | N |
| ATOM | 1346 | CA | GLN | A | 359 | 14.911 | 37.742 | 27.636 | 1.00 | 29.45 | C |
| ATOM | 1347 | C | GLN | A | 359 | 13.896 | 36.829 | 26.990 | 1.00 | 29.62 | C |
| ATOM | 1348 | O | GLN | A | 359 | 13.020 | 37.271 | 26.253 | 1.00 | 30.30 | O |
| ATOM | 1349 | CB | GLN | A | 359 | 14.215 | 38.974 | 28.202 | 1.00 | 33.27 | C |
| ATOM | 1350 | CG | GLN | A | 359 | 15.094 | 39.792 | 29.129 | 1.00 | 37.29 | C |
| ATOM | 1351 | CD | GLN | A | 359 | 14.521 | 41.165 | 29.394 | 1.00 | 41.00 | C |
| ATOM | 1352 | NE2 | GLN | A | 359 | 15.350 | 42.191 | 29.241 | 1.00 | 40.77 | N |
| ATOM | 1353 | OE1 | GLN | A | 359 | 13.344 | 41.303 | 29.741 | 1.00 | 44.29 | O |
| ATOM | 1354 | N | PRO | A | 360 | 14.006 | 35.531 | 27.267 | 1.00 | 30.18 | N |
| ATOM | 1355 | CA | PRO | A | 360 | 13.131 | 34.481 | 26.747 | 1.00 | 28.82 | C |
| ATOM | 1356 | C | PRO | A | 360 | 11.668 | 34.639 | 27.146 | 1.00 | 28.73 | C |
| ATOM | 1357 | O | PRO | A | 360 | 11.350 | 34.829 | 28.318 | 1.00 | 27.52 | O |
| ATOM | 1358 | CB | PRO | A | 360 | 13.722 | 33.212 | 27.346 | 1.00 | 29.99 | C |
| ATOM | 1359 | CG | PRO | A | 360 | 15.138 | 33.575 | 27.605 | 1.00 | 32.78 | C |
| ATOM | 1360 | CD | PRO | A | 360 | 15.042 | 34.958 | 28.139 | 1.00 | 29.61 | C |
| ATOM | 1361 | N | TRP | A | 361 | 10.788 | 34.544 | 26.159 | 1.00 | 28.63 | N |
| ATOM | 1362 | CA | TRP | A | 361 | 9.361 | 34.633 | 26.393 | 1.00 | 26.49 | C |
| ATOM | 1363 | C | TRP | A | 361 | 8.921 | 33.308 | 27.000 | 1.00 | 25.84 | C |
| ATOM | 1364 | O | TRP | A | 361 | 9.533 | 32.262 | 26.755 | 1.00 | 24.90 | O |
| ATOM | 1365 | CB | TRP | A | 361 | 8.638 | 34.894 | 25.073 | 1.00 | 25.57 | C |
| ATOM | 1366 | CG | TRP | A | 361 | 9.077 | 34.004 | 23.953 | 1.00 | 25.54 | C |
| ATOM | 1367 | CD1 | TRP | A | 361 | 8.677 | 32.723 | 23.714 | 1.00 | 25.38 | C |
| ATOM | 1368 | CD2 | TRP | A | 361 | 9.984 | 34.343 | 22.898 | 1.00 | 23.73 | C |
| ATOM | 1369 | CE2 | TRP | A | 361 | 10.084 | 33.215 | 22.054 | 1.00 | 22.93 | C |
| ATOM | 1370 | CE3 | TRP | A | 361 | 10.726 | 35.488 | 22.587 | 1.00 | 22.21 | C |
| ATOM | 1371 | NE1 | TRP | A | 361 | 9.273 | 32.243 | 22.574 | 1.00 | 22.24 | N |
| ATOM | 1372 | CZ2 | TRP | A | 361 | 10.895 | 33.201 | 20.909 | 1.00 | 22.49 | C |
| ATOM | 1373 | CZ3 | TRP | A | 361 | 11.534 | 35.473 | 21.446 | 1.00 | 20.83 | C |
| ATOM | 1374 | CH2 | TRP | A | 361 | 11.611 | 34.337 | 20.627 | 1.00 | 20.46 | C |
| ATOM | 1375 | N | ASP | A | 362 | 7.868 | 33.345 | 27.802 | 1.00 | 25.75 | N |
| ATOM | 1376 | CA | ASP | A | 362 | 7.392 | 32.130 | 28.446 | 1.00 | 25.24 | C |
| ATOM | 1377 | C | ASP | A | 362 | 7.009 | 31.057 | 27.436 | 1.00 | 25.91 | C |
| ATOM | 1378 | O | ASP | A | 362 | 6.433 | 31.347 | 26.387 | 1.00 | 26.71 | O |
| ATOM | 1379 | CB | ASP | A | 362 | 6.215 | 32.459 | 29.366 | 1.00 | 26.34 | C |
| ATOM | 1380 | CG | ASP | A | 362 | 6.641 | 33.275 | 30.579 | 1.00 | 25.52 | C |
| ATOM | 1381 | OD1 | ASP | A | 362 | 6.221 | 34.438 | 30.720 | 1.00 | 26.91 | O |
| ATOM | 1382 | OD2 | ASP | A | 362 | 7.410 | 32.745 | 31.393 | 1.00 | 29.84 | O |
| ATOM | 1383 | N | GLY | A | 363 | 7.361 | 29.816 | 27.758 | 1.00 | 25.34 | N |
| ATOM | 1384 | CA | GLY | A | 363 | 7.053 | 28.690 | 26.900 | 1.00 | 27.05 | C |
| ATOM | 1385 | C | GLY | A | 363 | 8.003 | 28.471 | 25.736 | 1.00 | 28.45 | C |
| ATOM | 1386 | O | GLY | A | 363 | 7.860 | 27.492 | 24.996 | 1.00 | 30.61 | O |
| ATOM | 1387 | N | LEU | A | 364 | 8.972 | 29.368 | 25.565 | 1.00 | 26.02 | N |
| ATOM | 1388 | CA | LEU | A | 364 | 9.921 | 29.238 | 24.464 | 1.00 | 24.76 | C |
| ATOM | 1389 | C | LEU | A | 364 | 10.452 | 27.817 | 24.383 | 1.00 | 24.90 | C |
| ATOM | 1390 | O | LEU | A | 364 | 10.450 | 27.197 | 23.315 | 1.00 | 23.61 | O |
| ATOM | 1391 | CB | LEU | A | 364 | 11.083 | 30.231 | 24.619 | 1.00 | 21.94 | C |
| ATOM | 1392 | CG | LEU | A | 364 | 12.144 | 30.212 | 23.506 | 1.00 | 23.73 | C |
| ATOM | 1393 | CD1 | LEU | A | 364 | 12.952 | 31.515 | 23.507 | 1.00 | 21.22 | C |
| ATOM | 1394 | CD2 | LEU | A | 364 | 13.062 | 29.008 | 23.700 | 1.00 | 20.56 | C |
| ATOM | 1395 | N | ASP | A | 365 | 10.901 | 27.293 | 25.513 | 1.00 | 24.56 | N |
| ATOM | 1396 | CA | ASP | A | 365 | 11.437 | 25.943 | 25.543 | 1.00 | 28.01 | C |
| ATOM | 1397 | C | ASP | A | 365 | 10.391 | 24.883 | 25.180 | 1.00 | 28.31 | C |
| ATOM | 1398 | O | ASP | A | 365 | 10.684 | 23.935 | 24.448 | 1.00 | 29.79 | O |
| ATOM | 1399 | CB | ASP | A | 365 | 12.026 | 25.665 | 26.929 | 1.00 | 30.55 | C |
| ATOM | 1400 | CG | ASP | A | 365 | 13.452 | 26.165 | 27.063 | 1.00 | 31.07 | C |
| ATOM | 1401 | OD1 | ASP | A | 365 | 13.762 | 27.274 | 26.585 | 1.00 | 30.27 | O |
| ATOM | 1402 | OD2 | ASP | A | 365 | 14.271 | 25.444 | 27.656 | 1.00 | 36.34 | O |
| ATOM | 1403 | N | GLU | A | 366 | 9.172 | 25.059 | 25.676 | 1.00 | 27.88 | N |
| ATOM | 1404 | CA | GLU | A | 366 | 8.086 | 24.120 | 25.421 | 1.00 | 28.12 | C |
| ATOM | 1405 | C | GLU | A | 366 | 7.749 | 24.003 | 23.933 | 1.00 | 26.72 | C |
| ATOM | 1406 | O | GLU | A | 366 | 7.672 | 22.898 | 23.395 | 1.00 | 24.63 | O |
| ATOM | 1407 | CB | GLU | A | 366 | 6.845 | 24.555 | 26.208 | 1.00 | 30.51 | C |
| ATOM | 1408 | CG | GLU | A | 366 | 5.669 | 23.608 | 26.107 | 1.00 | 36.88 | C |
| ATOM | 1409 | CD | GLU | A | 366 | 4.590 | 23.888 | 27.148 | 1.00 | 42.19 | C |
| ATOM | 1410 | OE1 | GLU | A | 366 | 3.631 | 23.083 | 27.240 | 1.00 | 44.13 | O |
| ATOM | 1411 | OE2 | GLU | A | 366 | 4.698 | 24.910 | 27.871 | 1.00 | 45.67 | O |
| ATOM | 1412 | N | HIS | A | 367 | 7.540 | 25.148 | 23.281 | 1.00 | 25.42 | N |
| ATOM | 1413 | CA | HIS | A | 367 | 7.214 | 25.178 | 21.861 | 1.00 | 24.46 | C |
| ATOM | 1414 | C | HIS | A | 367 | 8.418 | 24.654 | 21.073 | 1.00 | 25.00 | C |
| ATOM | 1415 | O | HIS | A | 367 | 8.276 | 23.913 | 20.097 | 1.00 | 22.88 | O |
| ATOM | 1416 | CB | HIS | A | 367 | 6.891 | 26.613 | 21.389 | 1.00 | 27.48 | C |
| ATOM | 1417 | CG | HIS | A | 367 | 5.797 | 27.299 | 22.157 | 1.00 | 27.40 | C |
| ATOM | 1418 | CD2 | HIS | A | 367 | 5.647 | 28.597 | 22.520 | 1.00 | 26.73 | C |
| ATOM | 1419 | ND1 | HIS | A | 367 | 4.641 | 26.661 | 22.553 | 1.00 | 26.53 | N |
| ATOM | 1420 | CE1 | HIS | A | 367 | 3.826 | 27.532 | 23.121 | 1.00 | 24.38 | C |
| ATOM | 1421 | NE2 | HIS | A | 367 | 4.413 | 28.714 | 23.112 | 1.00 | 27.39 | N |
| ATOM | 1422 | N | SER | A | 368 | 9.614 | 25.053 | 21.494 | 1.00 | 24.56 | N |
| ATOM | 1423 | CA | SER | A | 368 | 10.816 | 24.591 | 20.816 | 1.00 | 24.67 | C |
| ATOM | 1424 | C | SER | A | 368 | 10.850 | 23.049 | 20.839 | 1.00 | 24.51 | C |
| ATOM | 1425 | O | SER | A | 368 | 11.166 | 22.420 | 19.821 | 1.00 | 25.22 | O |
| ATOM | 1426 | CB | SER | A | 368 | 12.056 | 25.209 | 21.477 | 1.00 | 23.92 | C |
| ATOM | 1427 | OG | SER | A | 368 | 13.253 | 24.722 | 20.914 | 1.00 | 27.64 | O |
| ATOM | 1428 | N | GLN | A | 369 | 10.486 | 22.440 | 21.973 | 1.00 | 23.92 | N |
| ATOM | 1429 | CA | GLN | A | 369 | 10.477 | 20.965 | 22.086 | 1.00 | 24.64 | C |
| ATOM | 1430 | C | GLN | A | 369 | 9.414 | 20.329 | 21.191 | 1.00 | 24.55 | C |
| ATOM | 1431 | O | GLN | A | 369 | 9.672 | 19.337 | 20.509 | 1.00 | 23.42 | O |
| ATOM | 1432 | CB | GLN | A | 369 | 10.211 | 20.499 | 23.527 | 1.00 | 24.03 | C |
| ATOM | 1433 | CG | GLN | A | 369 | 10.089 | 18.962 | 23.656 | 1.00 | 26.78 | C |
| ATOM | 1434 | CD | GLN | A | 369 | 9.870 | 18.488 | 25.091 | 1.00 | 31.73 | C |
| ATOM | 1435 | NE2 | GLN | A | 369 | 10.715 | 17.568 | 25.545 | 1.00 | 30.77 | N |
| ATOM | 1436 | OE1 | GLN | A | 369 | 8.946 | 18.945 | 25.782 | 1.00 | 32.76 | O |
| ATOM | 1437 | N | ASP | A | 370 | 8.214 | 20.897 | 21.223 | 1.00 | 24.16 | N |
| ATOM | 1438 | CA | ASP | A | 370 | 7.117 | 20.386 | 20.420 | 1.00 | 26.15 | C |
| ATOM | 1439 | C | ASP | A | 370 | 7.480 | 20.468 | 18.939 | 1.00 | 24.54 | C |
| ATOM | 1440 | O | ASP | A | 370 | 7.365 | 19.489 | 18.200 | 1.00 | 22.63 | O |
| ATOM | 1441 | CB | ASP | A | 370 | 5.855 | 21.202 | 20.679 | 1.00 | 29.14 | C |
| ATOM | 1442 | CG | ASP | A | 370 | 4.681 | 20.704 | 19.883 | 1.00 | 34.35 | C |
| ATOM | 1443 | OD1 | ASP | A | 370 | 4.227 | 19.568 | 20.149 | 1.00 | 35.30 | O |
| ATOM | 1444 | OD2 | ASP | A | 370 | 4.217 | 21.441 | 18.978 | 1.00 | 36.99 | O |
| ATOM | 1445 | N | LEU | A | 371 | 7.921 | 21.640 | 18.502 | 1.00 | 21.63 | N |
| ATOM | 1446 | CA | LEU | A | 371 | 8.304 | 21.787 | 17.104 | 1.00 | 21.23 | C |
| ATOM | 1447 | C | LEU | A | 371 | 9.374 | 20.794 | 16.687 | 1.00 | 21.51 | C |
| ATOM | 1448 | O | LEU | A | 371 | 9.377 | 20.335 | 15.547 | 1.00 | 19.92 | O |
| ATOM | 1449 | CB | LEU | A | 371 | 8.810 | 23.189 | 16.834 | 1.00 | 21.87 | C |
| ATOM | 1450 | CG | LEU | A | 371 | 7.681 | 24.200 | 16.737 | 1.00 | 19.56 | C |
| ATOM | 1451 | CD1 | LEU | A | 371 | 8.290 | 25.572 | 16.779 | 1.00 | 19.41 | C |
| ATOM | 1452 | CD2 | LEU | A | 371 | 6.866 | 23.970 | 15.452 | 1.00 | 20.50 | C |
| ATOM | 1453 | N | SER | A | 372 | 10.281 | 20.469 | 17.605 | 1.00 | 20.28 | N |
| ATOM | 1454 | CA | SER | A | 372 | 11.347 | 19.531 | 17.297 | 1.00 | 21.04 | C |
| ATOM | 1455 | C | SER | A | 372 | 10.795 | 18.134 | 17.052 | 1.00 | 22.55 | C |
| ATOM | 1456 | O | SER | A | 372 | 11.317 | 17.393 | 16.218 | 1.00 | 19.63 | O |
| ATOM | 1457 | CB | SER | A | 372 | 12.375 | 19.481 | 18.441 | 1.00 | 19.21 | C |
| ATOM | 1458 | OG | SER | A | 372 | 12.994 | 20.743 | 18.603 | 1.00 | 17.52 | O |
| ATOM | 1459 | N | GLY | A | 373 | 9.739 | 17.782 | 17.788 | 1.00 | 23.62 | N |
| ATOM | 1460 | CA | GLY | A | 373 | 9.137 | 16.468 | 17.639 | 1.00 | 24.17 | C |
| ATOM | 1461 | C | GLY | A | 373 | 8.386 | 16.347 | 16.330 | 1.00 | 25.18 | C |
| ATOM | 1462 | O | GLY | A | 373 | 8.299 | 15.270 | 15.747 | 1.00 | 27.44 | O |
| ATOM | 1463 | N | ARG | A | 374 | 7.835 | 17.458 | 15.863 | 1.00 | 27.51 | N |
| ATOM | 1464 | CA | ARG | A | 374 | 7.097 | 17.448 | 14.613 | 1.00 | 28.96 | C |
| ATOM | 1465 | C | ARG | A | 374 | 8.054 | 17.375 | 13.433 | 1.00 | 28.09 | C |
| ATOM | 1466 | O | ARG | A | 374 | 7.771 | 16.698 | 12.443 | 1.00 | 28.97 | O |
| ATOM | 1467 | CB | ARG | A | 374 | 6.231 | 18.709 | 14.474 | 1.00 | 32.07 | C |
| ATOM | 1468 | CG | ARG | A | 374 | 5.238 | 18.945 | 15.607 | 1.00 | 35.47 | C |
| ATOM | 1469 | CD | ARG | A | 374 | 4.328 | 20.148 | 15.316 | 1.00 | 39.30 | C |
| ATOM | 1470 | NE | ARG | A | 374 | 3.487 | 20.481 | 16.466 | 1.00 | 44.09 | N |
| ATOM | 1471 | CZ | ARG | A | 374 | 2.512 | 21.388 | 16.460 | 1.00 | 47.26 | C |
| ATOM | 1472 | NH1 | ARG | A | 374 | 2.231 | 22.074 | 15.358 | 1.00 | 48.51 | N |
| ATOM | 1473 | NH2 | ARG | A | 374 | 1.811 | 21.612 | 17.567 | 1.00 | 48.82 | N |
| ATOM | 1474 | N | LEU | A | 375 | 9.187 | 18.068 | 13.531 | 1.00 | 25.48 | N |
| ATOM | 1475 | CA | LEU | A | 375 | 10.153 | 18.065 | 12.435 | 1.00 | 25.35 | C |
| ATOM | 1476 | C | LEU | A | 375 | 10.768 | 16.689 | 12.293 | 1.00 | 26.92 | C |
| ATOM | 1477 | O | LEU | A | 375 | 10.885 | 16.162 | 11.183 | 1.00 | 27.78 | O |
| ATOM | 1478 | CB | LEU | A | 375 | 11.251 | 19.106 | 12.666 | 1.00 | 19.68 | C |
| ATOM | 1479 | CG | LEU | A | 375 | 12.450 | 19.102 | 11.709 | 1.00 | 19.51 | C |
| ATOM | 1480 | CD1 | LEU | A | 375 | 11.982 | 19.204 | 10.258 | 1.00 | 19.25 | C |
| ATOM | 1481 | CD2 | LEU | A | 375 | 13.362 | 20.280 | 12.038 | 1.00 | 16.07 | C |
| ATOM | 1482 | N | ARG | A | 376 | 11.149 | 16.102 | 13.419 | 1.00 | 26.74 | N |
| ATOM | 1483 | CA | ARG | A | 376 | 11.753 | 14.788 | 13.382 | 1.00 | 29.15 | C |
| ATOM | 1484 | C | ARG | A | 376 | 10.795 | 13.768 | 12.772 | 1.00 | 28.35 | C |
| ATOM | 1485 | O | ARG | A | 376 | 11.234 | 12.823 | 12.137 | 1.00 | 25.80 | O |
| ATOM | 1486 | CB | ARG | A | 376 | 12.146 | 14.329 | 14.781 | 1.00 | 32.73 | C |
| ATOM | 1487 | CG | ARG | A | 376 | 12.782 | 12.950 | 14.768 | 1.00 | 36.63 | C |
| ATOM | 1488 | CD | ARG | A | 376 | 12.251 | 12.102 | 15.891 | 1.00 | 40.92 | C |
| ATOM | 1489 | NE | ARG | A | 376 | 12.579 | 10.690 | 15.701 | 1.00 | 44.99 | N |
| ATOM | 1490 | CZ | ARG | A | 376 | 12.207 | 9.965 | 14.648 | 1.00 | 45.89 | C |
| ATOM | 1491 | NH1 | ARG | A | 376 | 11.489 | 10.514 | 13.673 | 1.00 | 48.31 | N |
| ATOM | 1492 | NH2 | ARG | A | 376 | 12.551 | 8.688 | 14.571 | 1.00 | 47.00 | N |
| ATOM | 1493 | N | ALA | A | 377 | 9.493 | 13.961 | 12.972 | 1.00 | 27.76 | N |
| ATOM | 1494 | CA | ALA | A | 377 | 8.507 | 13.037 | 12.423 | 1.00 | 28.89 | C |
| ATOM | 1495 | C | ALA | A | 377 | 8.286 | 13.320 | 10.936 | 1.00 | 28.67 | C |
| ATOM | 1496 | O | ALA | A | 377 | 8.005 | 12.419 | 10.150 | 1.00 | 27.77 | O |
| ATOM | 1497 | CB | ALA | A | 377 | 7.187 | 13.149 | 13.188 | 1.00 | 26.88 | C |
| ATOM | 1498 | N | ILE | A | 378 | 8.398 | 14.585 | 10.563 | 1.00 | 29.35 | N |
| ATOM | 1499 | CA | ILE | A | 378 | 8.227 | 14.974 | 9.176 | 1.00 | 29.63 | C |
| ATOM | 1500 | C | ILE | A | 378 | 9.357 | 14.392 | 8.347 | 1.00 | 31.68 | C |
| ATOM | 1501 | O | ILE | A | 378 | 9.131 | 13.866 | 7.260 | 1.00 | 32.13 | O |
| ATOM | 1502 | CB | ILE | A | 378 | 8.250 | 16.496 | 9.023 | 1.00 | 26.98 | C |
| ATOM | 1503 | CG1 | ILE | A | 378 | 6.942 | 17.085 | 9.548 | 1.00 | 22.46 | C |
| ATOM | 1504 | CG2 | ILE | A | 378 | 8.503 | 16.872 | 7.567 | 1.00 | 25.95 | C |
| ATOM | 1505 | CD1 | ILE | A | 378 | 6.916 | 18.585 | 9.512 | 1.00 | 23.42 | C |
| ATOM | 1506 | N | LEU | A | 379 | 10.578 | 14.497 | 8.860 | 1.00 | 33.50 | N |
| ATOM | 1507 | CA | LEU | A | 379 | 11.734 | 13.979 | 8.148 | 1.00 | 36.71 | C |
| ATOM | 1508 | C | LEU | A | 379 | 11.702 | 12.456 | 8.139 | 1.00 | 40.55 | C |
| ATOM | 1509 | O | LEU | A | 379 | 11.949 | 11.832 | 7.106 | 1.00 | 42.53 | O |
| ATOM | 1510 | CB | LEU | A | 379 | 13.020 | 14.496 | 8.799 | 1.00 | 35.02 | C |
| ATOM | 1511 | CG | LEU | A | 379 | 13.619 | 15.838 | 8.316 | 1.00 | 35.23 | C |
| ATOM | 1512 | CD1 | LEU | A | 379 | 12.559 | 16.790 | 7.829 | 1.00 | 33.46 | C |
| ATOM | 1513 | CD2 | LEU | A | 379 | 14.405 | 16.471 | 9.454 | 1.00 | 31.58 | C |
| ATOM | 1514 | N | GLN | A | 380 | 11.367 | 11.867 | 9.285 | 1.00 | 43.12 | N |
| ATOM | 1515 | CA | GLN | A | 380 | 11.305 | 10.414 | 9.435 | 1.00 | 47.27 | C |
| ATOM | 1516 | C | GLN | A | 380 | 10.170 | 9.963 | 10.362 | 1.00 | 49.92 | C |
| ATOM | 1517 | O | GLN | A | 380 | 10.476 | 9.454 | 11.464 | 1.00 | 51.65 | O |
| ATOM | 1518 | CB | GLN | A | 380 | 12.646 | 9.889 | 9.971 | 1.00 | 45.95 | C |
| ATOM | 1519 | CG | GLN | A | 380 | 13.747 | 9.852 | 8.931 | 1.00 | 45.00 | C |
| ATOM | 1520 | CD | GLN | A | 380 | 15.085 | 9.411 | 9.492 | 1.00 | 44.09 | C |
| ATOM | 1521 | NE2 | GLN | A | 380 | 16.156 | 10.060 | 9.037 | 1.00 | 41.59 | N |
| ATOM | 1522 | OE1 | GLN | A | 380 | 15.160 | 8.485 | 10.312 | 1.00 | 41.01 | O |
| ATOM | 1523 | OXT | GLN | A | 380 | 8.987 | 10.124 | 9.981 | 1.00 | 52.33 | O |
| TER | 1524 | | GLN | A | 380 | | | | | | |
| ATOM | 1525 | O | HOH | S | 382 | 8.984 | 31.080 | 4.311 | 1.00 | 25.60 | O |
| ATOM | 1526 | O | HOH | S | 383 | 22.149 | 20.610 | 16.508 | 1.00 | 37.57 | O |
| ATOM | 1527 | O | HOH | S | 384 | 11.039 | 28.457 | 28.139 | 1.00 | 28.06 | O |
| ATOM | 1528 | O | HOH | S | 385 | 25.558 | 33.633 | 2.110 | 1.00 | 32.48 | O |
| ATOM | 1529 | O | HOH | S | 386 | 11.374 | 27.187 | 18.076 | 1.00 | 21.66 | O |
| ATOM | 1530 | O | HOH | S | 387 | 15.860 | 53.069 | 19.851 | 1.00 | 29.29 | O |
| ATOM | 1531 | O | HOH | S | 388 | 7.942 | 35.794 | 10.659 | 1.00 | 23.99 | O |
| ATOM | 1532 | O | HOH | S | 389 | 13.378 | 29.360 | 5.500 | 1.00 | 29.15 | O |
| ATOM | 1533 | O | HOH | S | 390 | 7.341 | 9.444 | 11.898 | 1.00 | 31.01 | O |
| ATOM | 1534 | O | HOH | S | 391 | 43.562 | 28.416 | 1.807 | 1.00 | 35.97 | O |
| ATOM | 1535 | O | HOH | S | 392 | 25.319 | 22.257 | 14.318 | 1.00 | 49.36 | O |
| ATOM | 1536 | O | HOH | S | 393 | 31.710 | 20.056 | 4.320 | 1.00 | 33.09 | O |
| ATOM | 1537 | O | HOH | S | 394 | 15.258 | 41.014 | 0.551 | 1.00 | 34.16 | O |
| ATOM | 1538 | O | HOH | S | 395 | 9.469 | 33.819 | 2.386 | 1.00 | 25.94 | O |
| ATOM | 1539 | O | HOH | S | 396 | 18.027 | 34.476 | 29.152 | 1.00 | 38.19 | O |
| ATOM | 1540 | O | HOH | S | 397 | 24.746 | 20.963 | 3.060 | 1.00 | 30.34 | O |
| ATOM | 1541 | O | HOH | S | 398 | 10.277 | 33.329 | 30.205 | 1.00 | 34.91 | O |
| ATOM | 1542 | O | HOH | S | 399 | 32.864 | 38.789 | −6.666 | 1.00 | 50.17 | O |
| ATOM | 1543 | O | HOH | S | 400 | 27.901 | 29.515 | 9.445 | 1.00 | 30.49 | O |
| ATOM | 1544 | O | HOH | S | 401 | 28.903 | 31.499 | 14.584 | 1.00 | 45.96 | O |
| ATOM | 1545 | O | HOH | S | 402 | 17.027 | 7.306 | 12.279 | 1.00 | 33.80 | O |
| ATOM | 1546 | O | HOH | S | 403 | 3.422 | 22.624 | 12.931 | 1.00 | 45.54 | O |
| TER | 1547 | | HOH | S | 403 | | | | | | |
| ATOM | 1548 | ZN | ZN | Z | 381 | 9.180 | 29.922 | −0.828 | 1.00 | 31.51 | Z |
| END | | | | | | | | | | | |

Another embodiment of the present disclosure relates to the information provided by the three-dimensional crystal structure of a human APOBEC protein, Apo3G-CD2, and other structure models of APOBEC proteins obtained by computer modeling that bear similarity with an Apo3G-CD2 monomer and have a root-mean-square deviation (RMSD) of 2.0. Additionally, yet another embodiment of the present disclosure relates to how the information provided by the three-dimensional Apo3G-CD2 crystal structure and models of other homologous APOBECS can be used for drug discovery. Since Apo3G-CD2 shares sufficient sequence and structural similarities to all the other homologues included in the APOBEC protein family, it can be used for homology modeling to obtain computer models of other APOBEC proteins. For example, Apo3G-CD2 shares a sequence homology of 43% and buried residue homology of 83% with the N-terminal catalytic domain of APOBEC-2. With the C-terminal catalytic domain of APOBEC-3G, APOBEC-2 shares a sequence homology of 46% and buried residue homology of 83%. The extent of homology between the two proteins indicates that the proteins are folded in a similar manner. Therefore, information provided by the Apo3G-CD2 crystal structure can be used to model the single domain APOBEC proteins (AID, APOBEC-1, APOBEC-3A, APOBEC-3C, APOBEC3H, APOBEC-4) and the double-domain APOBEC proteins (APOBEC3B, APOBEC-3DE, APOBEC3G and APOBEC3F).

Yet another embodiment of the present disclosure relates to the structural information pertaining to the unique features of an APOBEC active site, which is provided by the three-dimensional crystal structure of Apo3G-CD2 and other structure models of APOBEC proteins obtained by computer modeling that bear similarity with an Apo3G-CD2 monomer and have a root-mean-square deviation (RMSD) of 2.0.

Yet another embodiment of the present disclosure relates to the structural information pertaining to unique features of APOBEC oligomerization, which is provided by the three-dimensional crystal structure of Apo3G-CD2 and other structure models of APOBEC proteins obtained by computer modeling that bear similarity with an Apo3G-CD2 monomer and have a root-mean-square deviation (RMSD) of 2.0.

Yet another embodiment of the present disclosure relates to the structural information pertaining to the APOBEC residues which reside on the surface of APOBEC proteins, which is provided by the three-dimensional crystal structure of Apo3G-CD2 and other structure models of APOBEC proteins obtained by computer modeling that bear similarity with an Apo3G-CD2 monomer and have a root-mean-square deviation (RMSD) of 2.0.

Yet another embodiment of the present disclosure relates to a method for the identification of compounds which inhibit APOBEC DNA or RNA binding and Zinc coordination within the APOBEC active site. Such compounds could be used to prevent or treat aberrant cytidine deamination activity of APOBEC enzymes causing chronic diseases, such as B cell lymphomas. Additionally, such compounds could enhance the anti-viral action of APOBEC enzymes. It has been demonstrated that APOBEC3G and APOBEC3F are associated with inhibitory RNA molecules and/or inhibitory ribonucleoprotein complexes in cells that are targets for HIV infection (4). Releasing APOBEC3G or APOBEC3F from these RNA complexes with a drug that inhibits RNA binding, while DNA binding remains intact, could restore their post entry HIV viral restriction properties. In this case, APOBEC3G or APOBEC3F would be able to inactivate the HIV provirus by introducing extensive cytidine deaminations onto the viral cDNA.

Yet another embodiment of the present disclosure includes a method including one or more steps of: (1) providing a three dimensional structure of an APOBEC protein or a model of a homologous APOBEC protein; and, (2) identifying a candidate compound that can affect DNA or RNA binding or zinc coordination within the APOBEC active sites via structure based drug design utilizing structural information provided in (1). The three dimensional structure of Apo3G-CD2 or a model(s) of homologous APOBEC proteins includes structures: (a) defined by atomic coordinates of a three dimensional structure of a crystalline Apo3G-CD2 protein with the atomic coordinates represented in table 1 (monomer); (b) defined by atomic coordinates wherein at least 50% of the structure has an average root-mean-square deviation (RMSD) from backbone atoms in the secondary structure elements represented by the atomic coordinates of (a) of equal to or less than about 2.5 Å for main chain Ca carbon backbone; and (c) a structure defined by atomic coordinates derived from Apo3G-CD2 molecules arranged in a crystalline manner in a space group C2 so as to form a unit cell of dimensions: a=83.464 Å, b=57.329 Å, c=40.5787 Å and α=90°, β=96.46°, γ=90°.

In another aspect of this embodiment, the methods described above further includes the step (3) of screening lead compounds identified in step (2) that inhibit the binding of an APOBEC protein to DNA, RNA or zinc. The step (3) of screening can include: (a) contacting the candidate compound identified in step (2) with an APOBEC protein or a fragment thereof or with the APOBEC substrates (DNA, RNA or zinc) under conditions in which the APOBEC protein can bind its substrate in the absence of the candidate compound; and (b) measuring the binding affinity of the APOBEC protein or fragment thereof to its substrates (DNA, RNA or zinc); wherein a candidate inhibitor compound is selected as a compound that inhibits the binding of the APOBEC protein to its substrate when there is a decrease in the binding affinity of the APOBEC protein or fragment thereof to its substrate (DNA,RNA or zinc), as compared to in the absence of the candidate inhibitor compound.

Another embodiment of the present disclosure relates to a method for the identification of compounds which enhance the ability of the APOBEC protein to bind DNA or RNA. Such compounds could potentially restore the function of AID in patients diagnosed with Hyper-IgM-2 syndrome. A subset of these patients has mutations in the gene encoding for AID that may impair DNA binding. Compounds that enhance the DNA binding capabilities of AID could potentially correct this defect. Additionally, these compounds may enhance the anti-viral properties of the APOBEC enzymes. This method includes the steps of: (1) providing a three dimensional structure of an APOBEC protein or a model of a homologous APOBEC protein as described in detail above; and, (2) identifying a candidate compound that can enhance DNA or RNA binding via structure based drug design utilizing structural information provided in (1). The step (3) of screening can include: (a) contacting the candidate compound identified in step (2) with an APOBEC protein or a fragment thereof or with the APOBEC substrates, DNA or RNA, under conditions in which the APOBEC protein can bind its substrate in the absence of the candidate compound; and (b) measuring the binding affinity of the APOBEC protein or fragment thereof to its substrates (DNA or RNA); wherein a lead compound is selected as a compound that enhances the binding of the APOBEC protein to its substrate (DNA or RNA) when there is an increase in the binding affinity of the APOBEC protein or fragment thereof to its substrate (DNA or RNA), as compared to in the absence of the lead compound.

Yet another embodiment of the present disclosure relates to a method for the identification of compounds which disrupt APOBEC protein oligomerization. Such compounds could be used to prevent or treat aberrant cytidine deamination activity of APOBEC enzymes causing chronic diseases, such as B cell lymphomas. Experimental evidence has been reported which suggests that APOBEC oligomerization can alter its deamination activity. Yet another embodiment related to a method including one or more of the steps of: (1) providing a three dimensional structure of an APOBEC protein or a model of a homologous APOBEC protein as described in detail above; and, (2) identifying a candidate compound that can disrupt oligomerization (for example, dimerization or tetramerization) via structure based drug design utilizing structural information provided in (1). The step (3) of screening can include: (a) contacting the candidate compound identified in step (2) with an APOBEC protein or a fragment thereof under conditions in which the APOBEC protein can oligomerize in the absence of the candidate compound; and (b) measuring the oligomerization of the APOBEC protein or fragment thereof; wherein a candidate inhibitor compound is selected as a compound that inhibits the oligomerization of the APOBEC protein when there is a decrease in the oligomerization of the APOBEC protein or fragment thereof, as compared to in the absence of the candidate inhibitor compound. APOBEC oligomerization can be measured by many techniques including, but not limited to: gel filtration, dynamic light scattering, native gel analysis, protein cross linking, immunoprecipitation, FRET analysis or BIACore.

Yet another embodiment of the present disclosure relates to a method for the identification of compounds which enhance APOBEC protein oligomerization. Such compounds could be used to enhance the anti-viral activity of the APOBEC enzymes by increasing DNA deamination activity and RNA binding to the viral RNA. Further, such compounds could be used to repair the effects of mutations in the AID protein which disrupt AID oligomerization and cause Hyper-IgM-2 syndrome. In one aspect of the present disclosure, this method includes the steps of: (1) providing a three dimensional structure of an APOBEC protein or a model of a homologous APOBEC protein as described in detail above; and, (2) identifying a candidate compound that can enhance oligomerization (for example, dimerization or tetramerization) via structure based drug design utilizing structural information provided in (1). The step (3) of screening can include: (a) contacting the candidate compound identified in step (2) with an APOBEC protein or a fragment thereof under conditions in which the APOBEC protein can oligomerize in the absence of the candidate compound; and (b) measuring the oligomerization of the APOBEC protein or fragment thereof; wherein a lead compound is selected as a compound that enhances the oligomerization of the APOBEC protein when there is an increase in the oligomerization of the APOBEC protein or fragment thereof, as compared to in the absence of the lead compound. APOBEC oligomerization can be measured by many techniques including but not limited to: gel filtration, dynamic light scattering, native gel analysis, protein cross linking, immunoprecipitation, FRET analysis or BIACore.

Yet another embodiment of the present disclosure relates to a method for the identification of compounds which inhibit HIV viral infectivity factor (Vif) protein from binding to an APOBEC protein. The HIV Vif protein can bind to most all of the APOBEC enzymes regardless of their ability to restrict HIV replication. For example, Vif can bind to AID and inhibit its deamination activity. In cells that are targets for HIV infection, Vif binds to APOBEC3G and APOBEC3F and targets it for ubiquitylation and proteasome mediated degradation. Compounds that can disrupt Vif and APOBEC protein interactions may serve as very effective anti-viral drugs.

In one aspect of the method described above, the steps include one or more of the following: (1) providing a three dimensional structure of an APOBEC protein or a model of a homologous APOBEC protein as described in detail above; and, (2) identifying a candidate compound that can disrupt Vif and APOBEC binding interactions via structure based drug design utilizing structural information provided in (1). The step (3) of screening can include: (a) contacting the candidate compound identified in step (2) with an APOBEC protein or a fragment thereof, or with Vif or a fragment thereof, under conditions in which the APOBEC protein and Vif can interact in the absence of the candidate compound; and (b) measuring the binding interactions of the APOBEC protein or fragment thereof with Vif or a fragment thereof; wherein a lead inhibitory compound is selected when there is a decrease in the binding interactions of the APOBEC protein or fragment thereof with Vif or a fragment thereof, as compared to in the absence of the lead compound.

Yet another embodiment of the present disclosure relates to a method for the identification of compounds which inhibit APOBEC ubiquitylation and proteasomal mediated degradation. In cells that are targets for HIV infection, Vif binds to APOBEC3G and APOBEC3F and targets it for ubiquitylation and proteasomal mediated degradation. Compounds that can disrupt APOBEC ubiquitlyation may serve as very effective anti-viral drugs. In one aspect of the methods described above, the method includes one or more of the steps of: (1) providing a three dimensional structure of an APOBEC protein or a model of a homologous APOBEC protein as described in detail above; and, (2) identifying a candidate compound that can disrupt Vif and APOBEC binding interactions via structure based drug design utilizing structural information provided in (1). The step (3) of screening can include: (a) contacting the candidate compound identified in step (2) with an APOBEC protein or a fragment thereof under conditions in which the APOBEC protein or a fragment thereof becomes ubiquitylated in the absence of the candidate compound; and (b) measuring the ubiquitlyation of the APOBEC protein of fragment thereof; wherein a lead inhibitory compound is selected when there is a decrease in ubiquitylation of the APOBEC protein or fragment thereof, as compared to in the absence of the lead compound. Ubiquitlyation can be measured by many techniques including, but not limited to: immunoprecipitation and western blot analysis with an antibody specific for ubiquitin and the APOBEC protein.

In yet another aspect of various embodiments of the present disclosure, the step (2) of identifying a compound in the method described above in this present disclosure can include any suitable method of drug design, drug screening or identification, including, but not limited to: directed drug design, random drug design, grid-based drug design, and/or computational screening of one or more databases of chemical compounds.

Yet another embodiment of the present disclosure relates to a method for preparing APOBEC proteins having modified biological activity. In one embodiment, the method includes the steps of: (1) providing a three dimensional structure of an APOBEC protein or a model of a homologous APOBEC protein as described in detail above; (2) utilizing the structural information provided by (1) to identify at least one or more sites in the structure contributing to the biological activity of an APOBEC protein; and (3) modifying at least one or more sites in an APOBEC protein to alter its biological activity. The mutant APOBEC protein comprises an amino acid sequence that differs from the wildtype sequence via amino acid substitutions. The APOBEC mutant protein includes mutations that can inhibit, reduce or enhance oligomerization, zinc coordination, binding to DNA or RNA substrates, binding to cellular co-factors or viral proteins including but not limited to HIV Vif, as compared to the wild-type APOBEC protein.

Yet another embodiment of the present disclosure includes a method for producing crystals of APOBEC-2. Native and selenium-methionine labeled protein is concentrated to 15 mg per ml in a buffer containing 25 mM Hepes, pH 7.0, 50 mM NaCl and 10 mM dithiothreitol. Crystals are grown at 18° C. by hanging-drop vapor diffusion from a reservoir solution of 85 mM Na-citrate, pH 5.6, 160 mM LiSO4, 24% (weight/volume) polyethylene glycol monomethyl ether and 15% glycerol.

Yet another embodiment of the present disclosure includes a representation, or model, of the three dimensional structure of an APOBEC protein, such as a computer model. A computer model of the present disclosure can be produced using any suitable software program, including, but not limited to, MOLSCRIPT 2.0 (Avatar Software AB, Heleneborgsgatan 21C, SE-11731 Stockholm, Sweden), the graphical display program 0 (Jones et. al., Acta Crystallography, vol. A47, p. 110, 1991), the graphical display program GRASP, or the graphical display program INSIGHT. Suitable computer hardware useful for producing an image of the present disclosure is known to those of skill in the art (e.g., a Silicon Graphics Workstation).

A representation, or model, of the three dimensional structure of the Apo3G-CD2or any other APOBEC protein for which a crystal has been produced can also be determined using techniques which include molecular replacement or SIR/MIR (single/multiple isomorphous replacement). Methods of molecular replacement are generally known by those of skill in the art (generally described in Brunger, Meth. Enzym., vol. 276, pp. 558-580, 1997; Navaza and Saludjian, Meth. Enzym., vol. 276, pp. 581-594, 1997; Tong and Rossmann, Meth. Enzym., vol. 276, pp. 594-611, 1997; and Bentley, Meth. Enzym., vol. 276, pp. 611-619, 1997, each of which are incorporated by this reference herein in their entirety) and are performed in a software program including, for example, AmoRe (CCP4, Acta Cryst. D50, 760-763 (1994) or XPLOR. Briefly, X-ray diffraction data is collected from the crystal of a crystallized target structure.

The X-ray diffraction data is transformed to calculate a Patterson function. The Patterson function of the crystallized target structure is compared with a Patterson function calculated from a known structure (referred to herein as a search structure). The Patterson function of the crystallized target structure is rotated on the search structure Patterson function to determine the correct orientation of the crystallized target structure in the crystal. The translation function is then calculated to determine the location of the target structure with respect to the crystal axes. Once the crystallized target structure has been correctly positioned in the unit cell, initial phases for the experimental data can be calculated. These phases are necessary for calculation of an electron density map from which structural differences can be observed and for refinement of the structure. Preferably, the structural features (e.g., amino acid sequence, conserved di-sulphide bonds, and β-strands or β-sheets) of the search molecule are related to the crystallized target structure.

In yet another embodiment of the present disclosure, a three dimensional structure of an Apo3G-CD2 homologue protein includes a structure represented by atomic coordinates, wherein at least 50% of the structure has an average root-mean-square deviation (RMSD) from backbone atoms in secondary structure elements the three dimensional structure represented by the atomic coordinates of Table 1 of equal to or less than about 1.0 Å. Such a structure can be referred to as a structural homologue of the APOBEC structures defined by Table 1. Preferably, at least 50% of the structure has an RMSD from backbone atoms in secondary structure elements in the three dimensional structure represented by the atomic coordinates of Table 1 of equal to or less than about 0.7 Å, equal to or less than about 0.5 Å, and most preferably, equal to or less than about 0.3 Å. In another embodiment, a three dimensional structure of an Apo3G-CD2 protein provided by the present disclosure includes a structure defined by atomic coordinates that define a three dimensional structure, wherein at least about 75% of such structure has the recited average RMSD value, and more preferably, at least about 90% of such structure has the recited average RMSD value, and most preferably, about 100% of such structure has the recited average RMSD value.

In yet another embodiment of the present disclosure, the RMSD of a structural homologue of Apo3G-CD2 can be extended to include atoms of amino acid side chains. As used herein, the phrase “common amino acid side chains” refers to amino acid side chains that are common to both the structural homologue and to the structure that is actually represented by such atomic coordinates. Preferably, at least 50% of the structure has an average RMSD from common amino acid side chains in the three dimensional structure represented by the atomic coordinates of Table 1 of equal to or less than about 1.0 Å equal to or less than about 0.7 Å, equal to or less than about 0.5 Å, and most preferably, equal to or less than about 0.3 Å. In a more preferred embodiment, a three dimensional structure of an Apo3G-CD2 protein provided by the present disclosure includes a structure defined by atomic coordinates that define a three dimensional structure, wherein at least about 75% of such structure has the recited average RMSD value, and more preferably, at least about 90% of such structure has the recited average RMSD value, and most preferably, about 100% of such structure has the recited average RMSD value.

Suitable structures and models useful for structure based drug design are disclosed herein. Preferred target structures to use in a method of structure based drug design include any representations of structures produced by any modeling method disclosed herein, including molecular replacement and fold recognition related methods.

According to the present disclosure, the step of designing a compound for testing in a method of structure based identification of the present disclosure can include creating a new chemical compound or searching databases of libraries of known compounds (e.g., a compound listed in a computational screening database containing three dimensional structures of known compounds). Designing can also be performed by simulating chemical compounds having substitute moieties at certain structural features. The step of designing can include selecting a chemical compound based on a known function of the compound. A preferred step of designing comprises computational screening of one or more databases of compounds in which the three dimensional structure of the compound is known and is interacted (e.g., docked, aligned, matched, interfaced) with the three dimensional structure of an APOBEC protein by computer (e.g. as described by Humblet and Dunbar, Animal Reports in Medicinal Chemistry, vol. 28, pp. 275-283, 1993, M Venuti, ed., Academic Press). Methods to synthesize suitable chemical compounds are known to those of skill in the art and depend upon the structure of the chemical being synthesized. Methods to evaluate the bioactivity of the synthesized compound depend upon the bioactivity of the compound (e.g., inhibitory or stimulatory) and are disclosed herein.

Various other methods of structure-based drug design are disclosed in Maulik et al., 1997, Molecular Biotechnology: Therapeutic Applications and Strategies, Wiley-Liss, Inc., which is incorporated herein by reference in its entirety. Maulik et al. disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In a molecular diversity strategy, large compound libraries are synthesized, for example, from peptides, oligonucleotides, carbohydrates and/or synthetic organic molecules, using biological, enzymatic and/or chemical approaches. The critical parameters in developing a molecular diversity strategy include subunit diversity, molecular size, and library diversity. The general goal of screening such libraries is to utilize sequential application of combinatorial selection to obtain high-affinity ligands for a desired target, and then to optimize the lead molecules by either random or directed design strategies. Methods of molecular diversity are described in detail in Maulik, et al., ibid.

Maulik et al. also disclose, for example, methods of directed design, in which the user directs the process of creating novel molecules from a fragment library of appropriately selected fragments; random design, in which the user uses a genetic or other algorithm to randomly mutate fragments and their combinations while simultaneously applying a selection criterion to evaluate the fitness of candidate ligands; and a grid-based approach in which the user calculates the interaction energy between three dimensional receptor structures and small fragment probes, followed by linking together of favorable probe sites.

In the present method of structure based drug design, it is not necessary to align a candidate chemical compound (i.e., a chemical compound being analyzed in, for example, a computational screening method of the present disclosure) to each residue in a target site (target sites will be discussed in detail below). Suitable candidate chemical compounds can align to a subset of residues described for a target site. Preferably, a candidate chemical compound comprises a conformation that promotes the formation of covalent or noncovalent crosslinking between the target site and the candidate chemical compound. Preferably, a candidate chemical compound binds to a surface adjacent to a target site to provide an additional site of interaction in a complex. When designing an antagonist (i.e., a chemical compound that inhibits the binding of a substrate for an APOBEC protein by blocking a binding site or interface), the antagonist should bind with sufficient affinity to the binding site or to substantially prohibit a substrate (i.e., a molecule that specifically binds to the target site) from binding to a target area. It will be appreciated by one of skill in the art that it is not necessary that the complementarity between a candidate chemical compound and a target site extend over all residues specified here in order to inhibit or promote binding of a ligand.

In general, the design of a chemical compound possessing stereochemical complementarity can be accomplished by techniques that optimize, chemically or geometrically, the “fit” between a chemical compound and a target site. Such techniques are disclosed by, for example, Sheridan and Venkataraghavan, Acc. Chem Res., vol. 20, p. 322, 1987: Goodford, J Med. Chem., vol. 27, p. 557, 1984; Beddell, Chem. Soc Reviews, vol. 279, 1985; Hol, Angew. Chem., vol. 25, p. 767, 1986; and Verlinde and Hol, Structure, vol. 2, p. 577, 1994, each of which are incorporated by this reference herein in their entirety.

One embodiment of the present disclosure for structure based drug design comprises identifying a chemical compound that complements the shape of an APOBEC protein, or a portion thereof. Such method is referred to herein as a “geometric approach”. In a geometric approach, the number of internal degrees of freedom (and the corresponding local minima in the molecular conformation space) is reduced by considering only the geometric (hard-sphere) interactions of two rigid bodies, where one body (the active site) contains pockets” or “grooves” that form binding sites for the second body (the complementing molecule, such as a ligand).

The geometric approach is described by Kuntz et al., J Mol. Biol., vol. 161, p. 269, 1982, which is incorporated by this reference herein in its entirety. The algorithm for chemical compound design can be implemented using the software program DOCK Package, Version 1.0 (available from the Regents of the University of California). Pursuant to the Kuntz algorithm, the shape of the cavity or groove on the surface of a structure (e.g., Apo3G-CD2) at a binding site or interface is defined as a series of overlapping spheres of different radii. One or more extant databases of crystallographic data (e.g., the Cambridge Structural Database System maintained by University Chemical Laboratory, Cambridge University, Lensfield Road, Cambridge CB2 1EW, U.K.) or the Protein Data Bank maintained by Brookhaven National Laboratory, is then searched for chemical compounds that approximate the shape thus defined. Chemical compounds identified by the geometric approach can be modified to satisfy criteria associated with chemical complementarity, such as hydrogen bonding, ionic interactions or Van der Waals interactions.

Yet another embodiment of the present disclosure for structure based identification of compounds comprises determining the interaction of chemical groups (“probes”) with an active site at sample positions within and around a binding site or interface, resulting in an array of energy values from which three dimensional contour surfaces at selected energy levels can be generated. This method is referred to herein as a “chemical-probe approach.” The chemical-probe approach to the design of a chemical compound of the present disclosure is described by, for example, Goodford, J Med Chem., vol. 28, p. 849, 1985, which is incorporated by this reference herein in its entirety, and is implemented using an appropriate software package, including for example, GRID (available from Molecular Discovery Ltd., Oxford 0X2 9LL, U.K.). The chemical prerequisites for a site-complementing molecule can be identified at the outset, by probing the active site of an APOBEC protein, with different chemical probes, e.g., water, a methyl group, an amine nitrogen, a carboxyl oxygen and/or a hydroxyl. Preferred sites for interaction between an active site and a probe are determined. Putative complementary chemical compounds can be generated using the resulting three dimensional pattern of such sites

According to the present disclosure, suitable candidate compounds to test using the method of the present disclosure include proteins, peptides or other organic molecules, and inorganic molecules. Suitable organic molecules include small organic molecules. Peptides refer to small molecular weight compounds yielding two or more amino acids upon hydrolysis. A polypeptide is comprised of two or more peptides. As used herein, a protein is comprised of one or more polypeptides. Preferred therapeutic compounds to design include peptides composed of “L” and/or “D” amino acids that are configured as normal or retroinverso peptides, peptidomimetic compounds, small organic molecules, or homo- or hetero-polymers thereof, in linear or branched configurations.

Preferably, a compound that is identified by the method of the present disclosure originates from a compound having chemical and/or stereochemical complementarity with an APOBEC protein. Such complementarity is characteristic of a compound that matches the surface of the protein either in shape or in distribution of chemical groups and binds to the APOBEC protein to promote or inhibit APOBEC ligand binding in a cell expressing an APOBEC protein upon the binding of the compound to the APOBEC protein. More preferably, a compound that binds to a ligand binding site of an APOBEC protein associates with an affinity of at least about 10-6 M, and more preferably with an affinity of at least about 10-7 M, and more preferably with an affinity of at least about 10-8 M.

Preferably, four general sites on an APOBEC protein are targets for structure based drug design (i.e., target sites), although other sites may become apparent to those of skill in the art. The four preferred sites include: (1) the interfaces between APOBEC monomers, dimers and tetramers; (2) the active site where zinc is coordinated and where cytosine to uracil deamination activity occurs on DNA or RNA substrates (3) the D128 residue on APOBEC3G or D118 on AID (4) and DNA or RNA binding sites. Combinations of any of these general sites are also suitable target sites.

The following discussion provides specific detail on compound identification (i.e., drug design) using target sites of APOBEC proteins based on the Apo3G-CD2 three-dimensional structure. It is to be understood, however, that one of skill in the art, using the description of the Apo3G-CD2 structure provided herein, will be able to identify compounds that are potential candidates for inhibiting, stimulating or enhancing the interaction of APOBEC proteins with their other substrates, cellular co-factors and other viral accessory proteins.

A candidate compound for binding to an APOBEC protein, including one of the preferred target sites described above, is identified by one or more of the methods of structure-based identification discussed above. As used herein, a “candidate compound” or “lead compound” refers to a compound that is selected by a method of structure-based identification described herein as having a potential for binding to an APOBEC protein (or its substrate) on the basis of a predicted conformational interaction between the candidate compound and the target site of the APOBEC protein. The ability of the candidate compound to actually bind to an APOBEC protein can be determined using techniques known in the art, as discussed in some detail below. A “putative compound” is a compound with an unknown regulatory activity, at least with respect to the ability of such a compound to bind to and/or regulate an APOBEC protein as described herein. Therefore, a library of putative compounds can be screened using structure based identification methods as discussed herein, and from the putative compounds, one or more candidate compounds for binding to an APOBEC protein can be identified. Alternatively, a candidate compound for binding to an APOBEC protein can be designed de novo using structure based drug design, also as discussed above. Candidate compounds can be selected based on their predicted ability to inhibit the binding of an APOBEC protein to its substrate, cellular co-factor or a viral accessory protein, such as HIV Vif and to disrupt or enhance the oligomerization of APOBEC monomers or dimers.

In accordance with the present disclosure, a cell-based assay is conducted under conditions which are effective to screen for candidate compounds useful in the method of the present disclosure. Effective conditions include, but are not limited to, appropriate media, temperature, pH and oxygen conditions that permit the growth of the cell that expresses the receptor. An appropriate, or effective, medium refers to any medium in which a cell that naturally or recombinantly expresses an APOBEC protein, when cultured, is capable of cell growth and expression of the APOBEC protein. Such a medium is typically a solid or liquid medium comprising growth factors and assimilable carbon, nitrogen and phosphate sources, as well as appropriate salts, minerals, metals and other nutrients, such as vitamins. Culturing is carried out at a temperature, pH and oxygen content appropriate for the cell. Such culturing conditions are within the expertise of one of ordinary skill in the art.

Cells that are useful in the cell-based assays of the present disclosure include any cell that expresses an APOBEC protein and particularly, other proteins that are associated with that APOBEC protein. Such cells include bacterial cells. Additionally, certain cells may be induced to express an APOBEC protein recombinantly. Therefore, cells that express an APOBEC protein can include cells that naturally express an APOBEC protein, recombinantly express an APOBEC protein, or which can be induced to express an APOBEC protein. Cells useful in some embodiments can also include cells that can express the HIV Vif protein, such as Hela or 293T cells.

The assay of the present disclosure can also be a non-cell based assay. In this embodiment, the candidate compound can be directly contacted with an isolated APOBEC protein or fragment of that APOBEC protein, and the ability of the candidate compound to bind to the APOBEC protein can be evaluated by a binding assay. The assay can, if desired, additionally include the step of further analyzing whether candidate compounds which bind to a portion of the APOBEC protein are capable of increasing or decreasing the activity of the APOBEC protein or disrupting its interactions with the HIV Vif protein. Such further steps can be performed by cell-based assay, as described above, or by non-cell-based assay.

Alternatively, soluble APOBEC protein may be recombinantly expressed and utilized in non-cell based assays to identify compounds that bind to APOBEC proteins. Recombinantly expressed APOBEC polypeptides or fusion proteins containing one or more extracellular domains of an APOBEC protein can be used in the non-cell based screening assays. In non-cell based assays the recombinantly expressed APOBEC protein is attached to a solid substrate by means well known to those in the art. For example, APOBEC3G and/or cell lysates containing such proteins can be immobilized on a substrate such as: artificial membranes, organic supports, biopolymer supports and inorganic supports. The protein can be immobilized on the solid support by a variety of methods including adsorption, cross-linking (including covalent bonding), and entrapment. Adsorption can be through van del Waal's forces, hydrogen bonding, ionic bonding, or hydrophobic binding. Exemplary solid supports for adsorption immobilization include polymeric adsorbents and ion-exchange resins. Solid supports can be in any suitable form, including in a bead form, plate form, or well form. The test compounds are then assayed for their ability to bind to an APOBEC protein and disrupt interactions with their substrates, cellular co-factors or viral accessory proteins such as HIV Vif.

Yet another embodiment of the present disclosure relates to a therapeutic composition that, when administered to an animal, inhibits or prevents the degradation of an APOBEC protein by proteasome mediated degradation. The therapeutic composition comprises a compound that inhibits the binding of HIV Vif protein to APOBEC3G or APOBEC3F. The method comprises: (a) providing a three dimensional structure or structure model of an APOBEC protein as previously described herein; (b) identifying a candidate compound for binding to the APOBEC protein by performing structure based drug design with the structure of (a) to identify a compound structure that binds to the three dimensional structure of the APOBEC protein; (c) synthesizing the candidate compound; and (d) selecting candidate compounds that inhibit HIV Vif binding to the APOBEC protein in the presence of the candidate compounds. Preferably, the compounds inhibit the formation of a complex between the APOBEC protein and HIV Vif.

Another embodiment of the present disclosure relates to a therapeutic composition that, when administered to an animal, inhibits or prevents the deamination activity of an APOBEC protein. One embodiment of the method comprises one or more of the following: (a) providing a three dimensional structure or structure model of an APOBEC protein as previously described herein; (b) identifying a candidate compound for binding to the APOBEC protein by performing structure based drug design with the structure of (a) to identify a compound structure that binds to the three dimensional structure of the APOBEC protein; (c) synthesizing the candidate compound; and (d) selecting candidate compounds that inhibit deamination activity of the APOBEC protein in the presence of the candidate compounds. Preferably, the compounds prevent or inhibit the formation of B cell lymphomas.

Methods of identifying candidate compounds and selecting compounds that bind to and activate or inhibit an APOBEC protein have been previously described herein. Candidate compounds can be synthesized using techniques known in the art, and depending on the type of compound. Synthesis techniques for the production of non-protein compounds, including organic and inorganic compounds are well known in the art.

For smaller peptides, chemical synthesis methods are preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3): 147-157; Bodanszky, 1985, Int. J. Pept. Protein Res. 25(5):449-474; H. Dugas and C. Penney, BIOORGANIC CHEMISTRY, (1981) at pages 54-92, all of which are incorporated herein by reference in their entirety. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture.

If larger quantities of a protein are desired, or if the protein is a larger polypeptide, the protein can be produced using recombinant DNA technology. A protein can be produced recombinantly by culturing a cell capable of expressing the protein (i.e., by expressing a recombinant nucleic acid molecule encoding the protein) under conditions effective to produce the protein, and recovering the protein. Effective culture conditions include, but are not limited to, effective media, bioreactor, temperature, pH and oxygen conditions that permit protein production. An effective medium refers to any medium in which a cell is cultured to produce the protein. Such medium typically comprises an aqueous medium having assimilable carbon, nitrogen and phosphate sources, and appropriate salts, minerals, metals and other nutrients, such as vitamins. Recombinant cells (i.e., cells expressing a nucleic acid molecule encoding the desired protein) can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art. Such techniques are well known in the art and are described, for example, in Sambrook et al., 1988, Molecular Cloning: A Laboratory Manual, Cold Spring Harbor Press, Cold Spring Harbor Laboratory, Cold Spring Harbor, N.Y. or Current Protocols in Molecular Biology (1989) and supplements.

As discussed above, a composition, and particularly a therapeutic composition, of the present disclosure generally includes the therapeutic compound (e.g., the compound identified by the structure based identification method) and a carrier, and preferably, a pharmaceutically acceptable carrier. Pharmaceutically acceptable carriers and preferred methods of administration of therapeutic compositions of the present disclosure have been described in detail above with regard to the administration of an inhibitor compound to a patient. Such carriers and administration protocols are applicable to this embodiment.

Another embodiment of the present disclosure relates to a computer for producing a three-dimensional model of a molecule or molecular structure, wherein the molecule or molecular structure comprises a three dimensional structure defined by atomic coordinates of Apo3G-CD2, or a three-dimensional model of a homologue of the molecule or molecular structure, wherein the homologue comprises a three dimensional structure that has an average root-mean-square deviation (RMSD) of equal to or less than about 2.0 Å for the backbone atoms in secondary structure elements in the Apo3G-CD2 protein, wherein the computer comprises: a) a computer-readable medium encoded with the atomic coordinates of the Apo3G-CD2 protein to create an electronic file; b) a working memory for storing a graphical display software program for processing the electronic file; c) a processor coupled to the working memory and to the computer-readable medium which is capable of representing the electronic file as the three dimensional model; and, d) a display coupled to the processor for visualizing the three dimensional model; wherein the three dimensional structure of the APOBEC protein is displayed on the computer.

### DETAILED DESCRIPTION

### Example 1

### The Crystal Structure of the Catalytic Domain of the Viral Restriction Factor APOBEC3G

The following example is put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e. g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec., second(s); min, minute (s); h or hr, hour(s); and the like.

Deamination Activity of the Apo3G-CD2

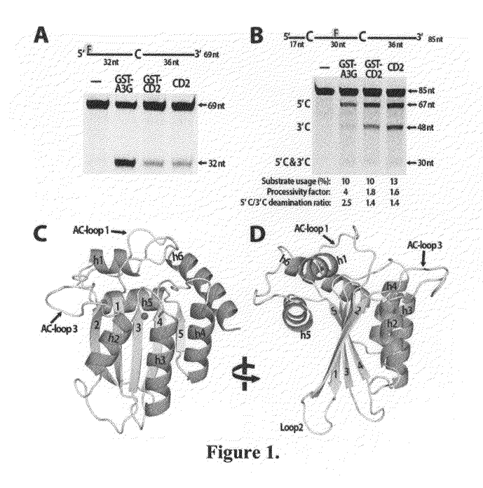

We have purified the human wild-type (wt) C-terminal cytidine deaminase domain of Apo3G (Apo3G-CD2, residues 197-380) expressed in E. coli, which is highly soluble and deaminates cytidine to uracil on ssDNA (FIG. 1A), with a specific activity (5 fmol/μg/min) that is about 25-fold lower than that of the full-length Apo3G (126 fmol/μg/min) (see Experimental Procedures). Full-length recombinant human Apo3G expressed in Sf9 insect cells acts processively on ssDNA with a 3′→45′ deamination bias (Chelico et al, 2008; Chelico et al., 2006). We analyzed the processive and polar properties of Apo3G-CD2 as well as the full-length Apo3G expressed in E. coli (FIG. 1B). Similar to the insect cell derived full length Apo3G, the full-length E. coli expressed Apo3G processively deaminates cytidine within two different 5′-CCC-3′ motifs located on a ssDNA substrate during one binding event (FIG. 1B). The full-length Apo3G also exerts a deamination bias by preferentially deaminating the cytidine in the CCC motif near the 5′-end of the ssDNA substrate (FIG. 1B). In contrast, the Apo3G-CD2 exhibits an approximate 2-fold decrease in processivity and polarity (FIG. 1B). These results indicate that Apo3G-CD2 partially retains several catalytic properties of the full-length Apo3G and that the CD1 domain in the context of the full-length Apo3G is most likely required for displaying the strong processive property and the 3′→5′ deamination bias on ssDNA.

Apo3G-CD2 Structure and Comparison to Other Cytidine Deaminases

The Apo3G-CD2 structure was solved through the multi-wavelength anomalous dispersion (MAD) phasing method using Se-Met diffraction data. The 2.3 Å resolution X-ray structure of the Apo3G-CD2 reveals a core β-sheet that is composed of five β-strands surrounded by six α-helices (FIGS. 1C and 1D). Helices 2-4 (h2-4) are packed alongside one face of the core β-sheet (FIG. 1C), while helix 1 (h1) and helix 5 (h5) are packed against the opposite face of the β-sheet (FIGS. 1C and 1D). Helix 6 (h6) is located at the edge of the β-sheet core, perpendicular to the β5 strand (FIG. 1C). Helix 4 (h4) makes extensive bonding contacts with h3 and h6, stabilizing the positions of those helices within Apo3G-CD2 (FIG. 1C).

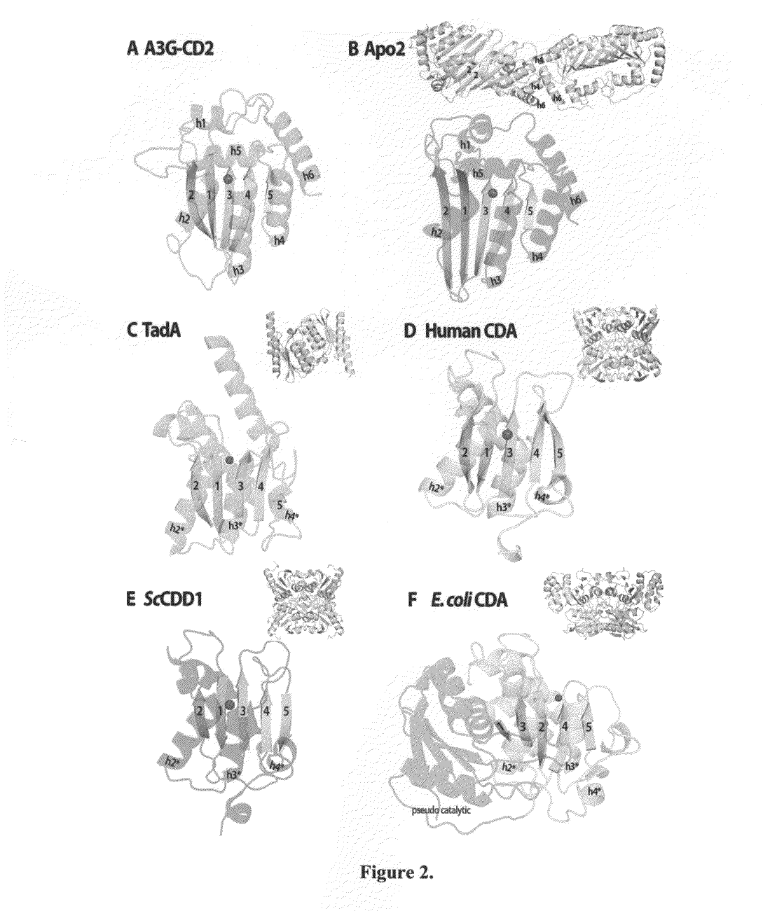

The Apo3G-CD2 structure shows similar core structural features as other cytidine deaminases within the superfamily of zinc-coordinating deaminases (Conticello et al., 2007b). All high resolution structures of cytidine deaminases have a typical core β-sheet consisting of five β-strands (FIGS. 2A-F). Additionally, these cytidine deaminase structures share a similar active site conformation with a zinc atom coordinated by three residues (two Cys and a His/Cys) from the second and the third α-helices (h2 and h3, FIGS. 2A-F) on the one side of the 5-stranded β-sheet core.

What differentiates the APOBEC structures from other known Zn-deaminase structures are the number and positions of the surrounding helices. The X-ray structures of A3G-CD2 and Apo2 have six surrounding helices that have the same spatial arrangement (FIG. 2A-B, 3A). The long helix 4 and helix 6 of Apo3G-CD2 and Apo2 are unique structural features that are absent from the other cytidine deaminases (FIG. 2A-F). While h6 is completely absent in the ECDA and the ScCDDi, the equivalent h4 forms a loop with one or two small 3₁₀ helices (labeled h4* in FIGS. 2C-F). In the ECDA, this h4* region connects the larger catalytic N-terminal domain with the smaller pseudo-catalytic domain at the C-terminus (FIG. 2F). Based on this ECDA structure, the Apo3G-CD2 helix 4 was previously modeled as a linker region that connects to a pseudo catalytic domain (Wedekind et al., 2003). In this model, both catalytic domains of the full-length Apo3G protein were predicted to have two linker regions and two pseudo-catalytic domains. However, the APOBEC structures clearly show that this predicted “linker” region forms a long helix 4 that is followed by the β5 strand, h5 and h6 before reaching the end of the domain. Furthermore, there is no pseudo catalytic domain equivalent to that of ECDA present in Apo3G or other APOBEC members (FIG. 2A-F) (Prochnow et al., 2007).

An analysis of the Zn-deaminase structures reveals that helices surrounding the β-sheet core dictate oligomerization and substrate access to the active site. The active forms of ECDA and ScCDDi are square-shaped dimers and tetramers with active sites that are buried between monomers and are only accessible to free base substrates (FIG. 2E-F, insets). In contrast, the h4 and h6 unique to Apo2 and Apo3G (FIG. 2A, 2B) sterically hinder the formation of a square-shaped dimer or tetramer. In Apo2, these helices (h4 and h6) direct the formation of an elongated tetramer with open active sites accessible to DNA or RNA (FIG. 2B, inset). Likewise, the h4 of Apo3G would make it sterically unlikely for the CDi and CD2 domains of full-length Apo3G to fold similar to an ECDA dimer or a ScCDDi tetramer (FIGS. 2A and 2E-F). Therefore, it is likely that the Apo3G CDi and CD2 domains fold in the same manner as an Apo2 dimer by pairing of the β2 strands (Zhang et al., 2007) (Figure B, inset). Similar to Apo2, interactions of the residues on h6 and h4 may facilitate the formation of an elongated A3G dimer (FIG. 2B, inset). Indeed, oligomers of Apo3G are observed using AFM (Chelico and Goodman, 2008), and small angle x-ray scattering data indicates that A3G dimers form elongated shapes (Chelico et al., 2008; Wedekind et al., 2006). Helices 4 and 6 on A3G-CD2 are nearly identical to those on Apo2. These helices (h4 and h6) are unique to the APOBEC structures and guide the elongated oligomerization so that the active sites are likely to be accessible to DNA and RNA substrates. Therefore, helices 4 and 6 appear to be a structural hallmark for all APOBEC family members.

Comparison of the Apo3G and Apo2 Structures

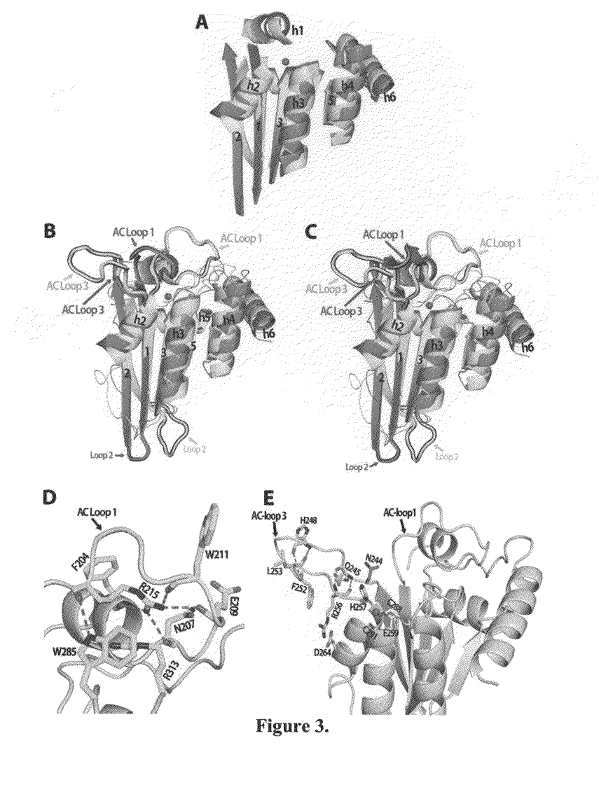

A superposition of the core structures of Apo3G-CD2 and Apo2 monomers exhibits substantial overlap for all six helices and for all five β-strands that are present in all Zn-deaminases (FIG. 3A), suggesting that the structures of APOBEC family members are highly conserved. However, the structural overlap reveals differences in the loops (FIGS. 3B and 3C). Two of these loops that differ dramatically from Apo2 are located around the active center (AC) and are referred to as AC-loops 1 and 3 (FIGS. 1C-D and FIGS. 3B-C), which could offer insight into why deamination activity is observed for Apo3G-CD2, but not for Apo2.

The AC-Loop 1, which connects h1 with β-strand 1, is located further away from the active site in Apo3G than in Apo2 (FIGS. 3B-C). The AC-loop 1 in Apo2 has two conformations (I and II) (Prochnow et al., 2007). In conformation I (cyan structure, FIG. 3B), the AC-loop 1 collapses over the active site due to a fourth coordination of E60 with the active site Zn, thereby effectively inhibiting DNA access to the active site. In conformation II (cyan structure, FIG. 3C), no coordination occurs between E60 and the active site Zn and the AC-loop 1 is pulled back from the active site (Prochnow et al., 2007). In contrast, the Apo3G AC-loop 1 lacks the equivalent “inhibitory” E60 residue in Apo2 that allows the loop to switch into a collapsed (closed) conformation over the active site (FIG. 3C). The open conformation of Apo3G AC-Loop 1 is stabilized by R215 through an elaborate hydrogen bond network with residues N207, E209, and W211 on the same loop, with F204 from h1, and with W285 near the active site Zn (FIG. 3E switch to 3D). Additional stabilization is provided by the hydrophobic packing of the long aliphatic chain of R215 with F204 and R313 (FIG. 3D). Through this extensive bonding network, R215 is critical for maintaining the open conformation of AC-loop 1 and for stabilizing the active site conformation via interactions with R313 and W285 located near the active site Zn (FIG. 3D). As shown in the section Apo3G Mutations Affecting DNA Binding and Deamination Activities below, we demonstrated that the R215E mutation in Apo3G abolishes deamination activity, consistent with a previous study (Chen et al., 2007), as does the corresponding R24E mutation in AID (Prochnow et al., 2007).

The Apo3G AC-loop 3, which connects the β2 strand with h2, is also located further away from the active site Zn than that of Apo2 (FIGS. 3B-C). This open conformation of the Apo3G AC-loop 3 is stabilized by hydrogen bonds between main-chain atoms of residues R256, F252, L253, H248 and Q245 within the loop (FIG. 3E). Additionally, the loop residue R256 interacts with D264 on a core helix via a strong salt bridge and it hydrophobically packs with another loop residue F252 via its long aliphatic chain (FIG. 3E). All these interactions stabilize the conformation of AC-loop 3 on which the active center residue H257 is located. As shown in the section Apo3G Mutations Affecting DNA Binding and Deamination Activities later, we demonstrated that R256E mutation of Apo3G reduced the deaminase activity greatly, suggesting an important role of R256 in maintaining the conformation of AC-loop 3 for deaminase activity. This result also suggests that the AC-loop 3 is not a flexible structure and that the conformation of the AC-loop 3 is important for deamination activity.

Comparison of the Apo3G-CD2 X-Ray Structure with the Apo3G-2K3A NMR Structure

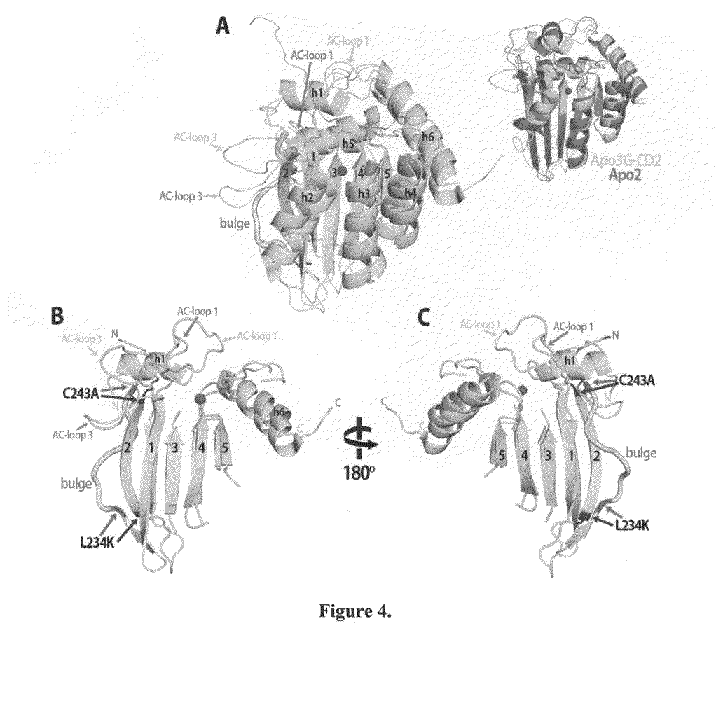

A recently reported NMR structure of an Apo3G CD2 mutant (called Apo3G-2K3A) resembles the X-ray structure of the wt Apo3G-CD2 (Chen et al., 2008). However, the structural superposition of the two structures reveals some significant differences (FIG. 4A). The overlay of the NMR and Apo3G X-ray structures gives a 4.8 A² RMSD, which is much larger than the 2.7 A² RMSD for the Apo3G X-ray and Apo2 structures where the most differences are on the loops (FIG. 4A, inset). These RMSD values indicate that the Apo3G X-ray structure differs more from the NMR structure than it does from the Apo2 structure. There are two notable differences revealed by the superposition between the X-ray and the NMR Apo3G structures. First, the N-terminal h1 that is predicted to be common to all APOBECs is absent from the NMR structure (FIGS. 4A-C). As a result, the NMR AC-loop 1 structure immediately following the absent h1 is positioned much closer to the active site. In this position, the NMR AC-loop 1 occupies part of the space that the AC-loop 3 occupies in the X-ray structure (FIG. 4A-C). The NMR Apo3G truncation is one residue shorter than our construct at the N-terminus and it is unclear if this shorter N-terminus can account for the loss of this helical structure. The second obvious and important difference between the X-ray and NMR and structures is the β2 strand (FIG. 4B-C). A loop-like structure (or bulge) in place of the β2 strand is presented in the NMR structure (PDB ID #2jyw, FIGS. 4B-C). In contrast, eight residues (235-243) in the Apo3G-CD2 X-ray structure form a stable β2 strand as part of the core β-sheet composed of five β-strands, which is also seen in the Apo2 and other cytidine deaminase structures surveyed from the available data base (FIGS. 2A-F). The β2 structure in Apo3G-CD2 is significant in that it will affect the conformation of the active center AC-loop 3 that connects directly to the β2 strand and will also influence predictions of how the two-domain full-length Apo3G monomer could fold and oligomerize, as will be explained in the section, “Models of Full-length Apo3G and Oligomerization.”

It should be noted that the NMR CD2 fragment (residue 198-384) carries five point mutations created to solve the protein solubility problem for the NMR study (Chen et al., 2008), whereas the A3G-CD2 protein (residue 197-380) reported here contains no mutations because this fragment is highly soluble as the wt sequence. Two of the five mutations in the NMR CD2 structure are located on both ends of the β2 strand (FIGS. 4B-C). Only one mutation, K234L near the start of the β2 strand, was reverse engineered to leucine to demonstrate that the loop-like bulge was not attributed to this mutation. However, the other C243A mutation located at the end of the β2 strand and right before AC-loop 3 could potentially affect the conformation of the β2 strand as well as the AC-loop 3 in the NMR structure. A similar β2 strand on a five-stranded β-sheet core is a structural feature that is observed in all wt cytidine deaminase structures available to date including: Apo2, and Apo3G-CD2 (FIG. 2A-F). Therefore, an intact full length β2 strand is likely to be the feature of wt Apo3G-CD2 and all other APOBEC proteins.

The Active Site of Apo3G-CD2

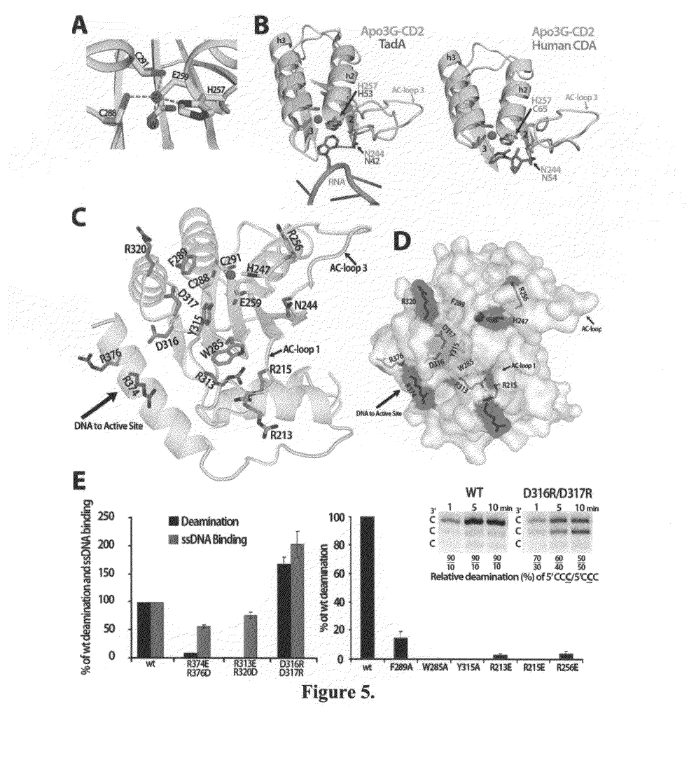

The deamination activity of Apo3G-CD2 involves a canonical type of zinc coordination where the active center Zn atom is coordinated by three residues His257, Cys288 and Cys291 and a water molecule located at a hydrogen bond distance from the Zn atom (FIG. 5A). This closely positioned water molecule can be activated to become a Zn-hydroxide for nucleophilic attack in the deamination reaction (Chung et al., 2005). The structure of the Apo3G-CD2 active center superimposes well with many of the free nucleotide deaminase structures (FIG. 5B). The AC-loops 1 and 3 of Apo3G-CD2 are positioned away from the active site to form an open conformation that provides ample space sufficient for fitting a large nucleic acid substrate (FIGS. 5C and 5D).

Surprisingly, the Apo3G AC-loop 3 and the two residues (N244 and H257) on this loop display a remarkable structural conservation with many distantly related Zn-deaminases, specifically TadA and hCDA (Chung et al., 2005; Losey et al., 2006) (FIG. 5B). The two equivalent TadA residues (N42 and H53) on a TadA loop (similar to the AC-loop 3) directly contact the target base of the RNA substrate. These residues overlap well with the Apo3G residues N244 and H257 on the AC-loop 3 when both structures are overlaid (FIG. 5B) (Losey et al., 2006). Similarly, two equivalent hCDA residues (N54 and C65) on a hCDA loop similar to the AC-loop 3 contact the substrate/inhibitor and overlap equally well with N244 and H257 on the AC-loop 3 of Apo3G (Chung et al., 2005) (FIG. 5B or C). Given the tight structural conservation of these Apo3G residues (N244 and H257) among other Zn-deaminases bound to their substrates, it is reasonable to suggest that these residues are also involved in DNA substrate binding. This type of structural conservation further suggests that the AC-loop 3 in the X-ray structure of Apo3G-CD2 is in a conformation ready to bind nucleic acid.

In all three enzymes, the conserved asparagine residue (Apo3G N244, TadA N42, hCDA N54) is located at the beginning of AC-loop 3 and immediately follows the last residue of the β2 strand (C243 in Apo3G) (FIG. 5B). Therefore, the 1.2 strand, especially the last few residues of the β2 strand, provides an anchoring point for the conserved asparagines and the AC-loop 3. Thus, it is conceivable that an intact β2 strand is important for positioning the AC-loop 3 in a proper conformation so that the conserved asparagines residue (Apo3G N244, TadA N42, hCDA N54) is positioned to interact with the target base during the deamination reaction with the active site Zn.

Structural Features Important for ssDNA Binding

In addition to the AC-loop 3 conformation and the residues N244 and H257 on the loop mentioned above, the Apo3G X-ray structure reveals other structural features for binding ssDNA substrate around the active site. First, a pocket generated by the open loop conformation around the active site has ample space to accommodate ssDNA (FIGS. 5C-D). Second, there are six positively charged residues around the active site pocket, R213, R215, R256, R313, R320, R374 and R376 (FIGS. 5C-D). Some of these residues are exposed and could make direct contact with ssDNA, while others are important for stabilizing the structure around the active center. Third, there are three evolutionarily conserved hydrophobic residues (W285, Y315 and F289) and a peculiar negatively charged residue (D317) located on the “floor” close to the active center (FIGS. 5C-D) that appear to be positioned for interacting with incoming ssDNA. The hydrophobic stacking of these residues with the bases could help orient the ssDNA substrate in the correct position relative to the active site Zn. This type of base stacking and positioning of the ssDNA into the active site may explain the A3G deamination specificity, in which cytidine deamination occurs predominantly at the 3′C in a 5′-CCC hotspot motif. The positively charged residues located at the periphery of the active center can bind the phosphate backbone of the ssDNA substrate. In an E. coli cell based deamination assay, mutations on the Apo3G-CD2 domain indicate that many of these residues disrupted deamination activity (Chen et al., 2007). In the following section, our data indicates that all of the full-length A3G mutants show defective deamination activity in an in-vitro assay using purified enzymes (FIG. 5E).

Apo3G Mutations Affecting DNA Binding and Deamination Activities

To correlate the structure and function of Apo3G, mutations of the residues predicted to be involved with binding DNA were constructed in the context of full-length Apo3G. The impact of these mutations on ssDNA binding and deamination activity was examined (FIGS. 5C-E). The positively charged arginines around the active site were mutated to either glutamic acid or aspartic acid. The deamination activity of these mutants was either abolished or significantly impaired (FIG. 5E). The R374 and R376 residues are positioned to interact with a negatively charged ssDNA phosphate backbone. Indeed, the ssDNA binding of the R374E/R376D double mutant is impaired by 46% in comparison to that of the wt Apo3G and the deamination activity is even more dramatically disrupted (FIG. 5E). The amino acid residue R213 on AC-loop 1 is structurally positioned to make contact with ssDNA and the point mutant R213E has only weak deamination activity (FIG. 5E).

The structure displays the hydrophobic residues W285 and Y315 on the floor of an open pocket and the F289 on the edge of the same open pocket. These residues could stack with the bases of ssDNA and position the DNA into the active site. The Apo3G mutants, W285A and Y315A, have no detectable deamination activity (FIG. 5E), which is consistent with a previous report (Chen et al., 2007) and the deamination activity of the F289A mutant is significantly impaired (FIG. 5E). Next to Y315 and W285 on the floor of the pocket, there are two negatively charged residues, D316/D317. The mutant D316R/1D317R displayed both higher ssDNA binding (2-fold) and deamination activity (1.6-fold). These enhanced activities could be caused by increasing the total positive charge near the active site (FIGS. 5C-E). Surprisingly, the D316R/D317R mutant also has altered substrate specificity. Unlike wt Apo3G that strongly favors deamination at the 3′C of a 5′CCC hot spot motif, the D316R/D317R mutant deaminates the middle and 3′C at about the same rate (FIG. 5E, inset). This result suggests that these negative residues in the wt Apo3G are important for orienting the substrate so that only the 3′C is positioned close to the active site Zn for deamination.

The structure reveals that some of the positively charged arginines (R256, R215 and R313) around the active site establish elaborate bonding networks and should play an important structural role by maintaining the proper conformation of the active center for DNA binding and deamination. Therefore it is not likely that these residues directly bind DNA. As discussed earlier, R256 plays a role in stabilizing the AC-loop 3 open conformation for substrate access through interactions with D264 and F252 (FIG. 3D). An R256E mutation would disrupt these interactions and dramatically impair deamination activity as observed (FIG. 5E). Similarly, the R215 residue is involved with extensive bonding networks that maintain the AC-loop 1 structure for substrate interactions (FIG. 3E). The R215E mutation most likely disrupts the AC-loop 1 structure resulting in the loss of the deamination activity (FIG. 5E). Previous mutagenesis data based on the NMR structure reported that the R313 residue is important for directly binding ssDNA (Chen et al., 2007). However, our data show that the R313E/R320D has only slightly impaired ssDNA binding, at 77% of wt levels, even though this mutant has no detectable deamination activity (FIG. 5E). The X-ray structure shows that the R313 residue is not accessible from the active site pocket for making direct contact with the ssDNA substrate. Instead, the long alphatic chain of the R313 packs with the W285 residue that is positioned directly in front of the active site. The mutation of the R313 residue most likely disrupts the position of W285, which may alter the positions of the DNA target base at active site thereby abolishing the deamination reaction.

DNA Binding Groove of Apo3G

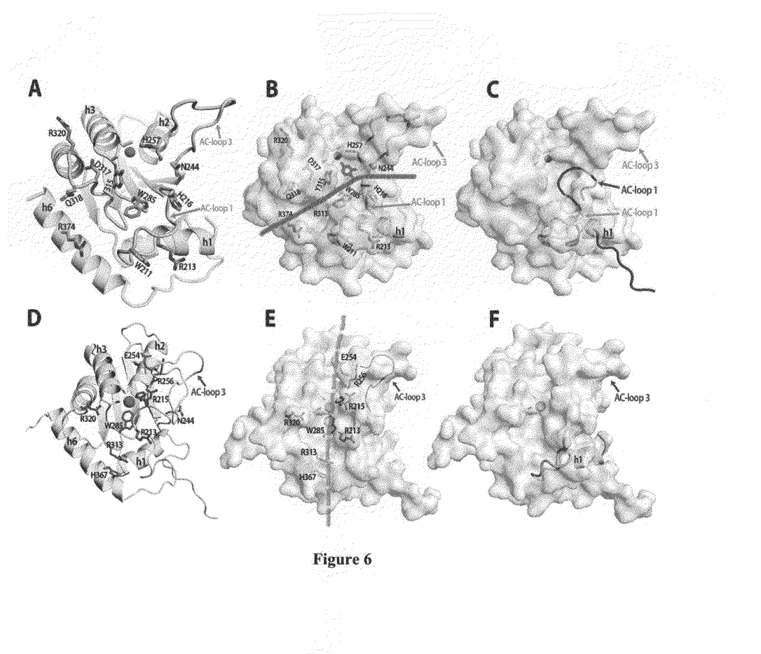

A surface representation of the Apo3G-CD2 X-ray structure reveals a spacious groove running across the active center pocket (FIGS. 6A-C). The structural features around the groove and our mutagenesis results suggest that the purpose of this groove is for binding ssDNA substrates. The groove starts between the AC-loop 1 and 3 on the right side of the displayed structure, leads into the deepest pocket next to the Zn atom, and continues toward the left side over helix 6 (FIGS. 6A and 6B). Aligned within this groove are polar and charged residues, from right to left, N244, H257, H216, R213, D317, Q318, and R374, which bind the incoming ssDNA (FIGS. 6A and 6B). Hydrophobic residues, Y315 and W285, positioned directly below the active site Zn could stack with the bases of the ssDNA and position and present the target cytidine to the Zn atom at the active center (FIG. 6B). As previously mentioned, two residues (N244 and H257) on the AC-loop 3 are conserved spatially and sequence-wise with the substrate-binding residues of distantly related Zn-deaminases, which suggests that they may directly contact the target base (FIG. 5B). The neighboring H216 from AC-loop 1 may base stack with a nearby base or contact the DNA phosphate backbone. In addition, W211, located on AC-loop 1 is in a solvent exposed position, which is unusual for a large hydrophobic residue that normally prefers the hydrophobic core of a molecule. In this position, it could potentially base stack with incoming ssDNA.

Molecular surface representation of the Apo3G-CD2 structure shows a small exposed area of the zinc atom from the pocket side (below the Zn), where the activating water molecule is located (FIG. 6B). As a result, positioning of the ssDNA within this groove allows for the correct orientation and angle of the cytidine base relative to the activated Zn hydroxide for deamination (FIG. 6B). This target base configuration relative to the Zn atom at the active site has been reported in other deaminase structures such as TadA and human cytidine deaminases (FIG. 5B) (Chung et al., 2005; Losey et al., 2006).

This DNA-binding groove model differs from the recently proposed ‘brim-domain” model based on the A3G-2K3A NMR structure (Chen et al., 2008). For ease of comparison, we maintained the same orientation previously used to describe the brim-domain model to present both the X-ray structure A3G-CD2 (FIGS. 6A-C) and the A3G-2K3A NMR structure (FIGS. 6D-F). All of the common structural features (h2, h3, h6, and the Zn atom) of both structures occupy the same position. In the brim-domain model, even though a groove is not defined, a proposed ssDNA binding path runs vertically between h2 and h3 and then over the Zn atom (FIG. 6E). This path is almost orthogonal to the “horizontal” ssDNA path proposed in our groove model (FIG. 6B).

Comparing the surface features of the X-ray structure (FIG. 6B) with the NMR structure (FIG. 6E) reveals that the horizontal groove is not present in the NMR structure, because the AC-loop 1 in the NMR structure occupies the groove space located near N244 of AC-loop 3 in the X-ray structure (FIGS. 6D and 6A). This position of the NMR AC-loop 1 completely blocks the open path of the groove that is seen in the X-ray structure (FIGS. 6C and 6F). As a result, the highly conserved N244 on AC-loop 3 of the NMR structure is displaced further away from the conserved spatial location that allows this residue to contact the target base (FIGS. 6D and 6A). For the deamination reaction to occur, the target base must be correctly positioned into the active site so that it is directed towards the active site Zn and coming in from the direction where the water molecule sits (FIG. 5A) (Chung et al., 2005; Losey et al., 2006). In the NMR brim domain model, the vertical path of ssDNA over the active site Zn does not permit the target base to flip into this correct orientation. Lastly, Chen et. al. (Chen et al., 2007) propose that the residues R313, R320, R213, and R215 form a positively charged brim around the active site for binding to the negatively charged ssDNA (FIG. 6D). In the A3G-CD2 crystal structure, the R313 and R215 are not accessible to the surface because both form an extensive bonding network with multiple surrounding residues that maintain the conformation of AC-loop 1 near the active site. Therefore, they are not likely to bind ssDNA. Additionally, the R320 residue in the X-ray structure is too far from the active site to make a contact with the incoming base as proposed based on the NMR structure. All of the key structural differences are attributable principally to the absence of helix 1 and of an intact β2 strand in the NMR structure, which dramatically alters the positions of these residues and the AC-loops 1 and 3 in comparison with the X-ray structure.

Models of Full-Length Apo3G and Oligomerization

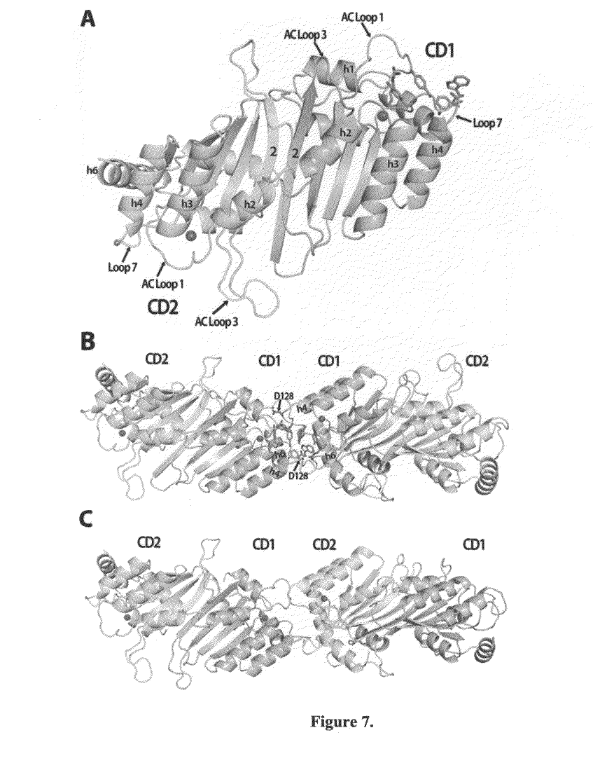

A full-length Apo3G structure containing both CD1 and CD2 domains can be modeled based on the close similarity of the Apo3G-CD2 structure with Apo2 (FIG. 3A). An even higher sequence similarity between Apo3G-CD1 and Apo3G-CD2 (Supplementary FIG. 1 showing alignment) strongly suggests that the structure of Apo3G-CD1 domain be similar to that of Apo3G-CD2 as well as Apo2 (also see Zhang et al., 2007). In the full length Apo3G, the CD1 and CD2 domains could interact with each other in the same way as two equivalent Apo2 monomers interact, i.e., by pairing their β2 strands to form a double domain structure (Zhang et al., 2007) (FIG. 7A). Two such double-domain Apo3G monomers may further dimerize through the inactive N-terminal CD1 domains (head-to-head) (FIG. 7B), which would resemble the tetramer of Apo2, where the active sites of the two monomers involved in tetramerization are in a “closed” inactive conformation (Conticello et al., 2007a; Wedekind et al., 2006). We cannot rule out the possibility that Apo3G could dimerize head-to-tail and/or tail-to-tail (FIG. 7C). However, residues at the tetramerization interface of Apo2 are highly conserved only in Apo3G-CD1 and not in Apo3G-CD2 (Supplementary Figure, residues marked by green dots). These conserved residues on A3G-CD1, R122, Y124, Y125, F126, and W127, would create a hydrophobic surface region on loop 7 that would pack together with the same hydrophobic CD1 region of another full-length Apo3G molecule (FIGS. 7A and 7B) to form a head-head (N—N) dimer. This dimeric formation via the Apo3G-CD1 domains could sterically obstruct the direct access of ssDNA to the active sites of CD1, but not of CD2. A previous report shows that the potential dimeric Apo3G residues, R122, Y124, Y125, F126 and W127, are required for virion incorporation and HIV-1 viral restriction (Huthoff and Malim, 2007). Notably, the D128 residue, which controls Apo3G species-specific interactions with HIV and SIV Vif proteins (Bogerd et al., 2004; Mangeat et al., 2004; Mariani et al., 2003; Xu et al., 2004), is located on loop 7 near this predicted dimeric interface of full-length Apo3G (FIG. 7B).

We have described the high-resolution structural features of Apo3G-CD2. The structure reveals that Apo3G-CD2 has the same core fold as Apo2 and other cytidine deaminases, all of which contain a β-sheet core composed of five β-strands. However, what differentiates the APOBEC structures from those of other zinc coordinating deaminases is the positioning of the surrounding helices and loops, which may account for some of the differences in assembly, substrate specificity, and regulation by other co-factors. The helices in Apo3G and Apo2 determine how the deaminase can oligomerize, which in turn influences how accessible the active site is to larger polynucleotide substrates. Both structures have a similar h4, h6 and a long β2 strand, of which the former two can prevent the canonical square-shaped oligomerization but facilitate an elongated oligomer formation. Furthermore, the X-ray structure of Apo3-CD2 reveals a deep groove across the active center, and mutagenesis has identified residues around this “substrate-groove” that play critical roles in substrate specificity, in ssDNA substrate binding, and in deaminase activity. The results of the Apo3G-CD2 structure and its analysis reported here will provide a basis to pursue further structural and functional studies of Apo3G and other APOBEC proteins that will facilitate our understanding of their important biological functions, such as how they interact with nucleic acid substrates for deamination, how their activity is regulated, and how they restrict HIV and other viral pathogens.

Protein Purification and Crystallization

Apo3G-CD2 was expressed and purified as a recombinant GST-fusion protein in Escherichia coli. Purified GST-fusion protein was digested by PreScission Protease. Further purification of the Apo3G-CD2 protein was completed with Superdex-75 gel filtration chromatography in 50 mM Hepes pH 7.0, 250 mM NaCl and 1 mM DTT. Native and selenium-methionine labeled protein were concentrated to 25 mg mL⁻¹. Crystals were grown at 18° C. by hanging-drop vapor diffusion from a reservoir solution of 100 mM MES pH 6.5, 40% PEG 200.

Structure Determination and Refinement

Selenium substituted methionine protein crystals were used for collecting Se-MAD data using the ALS synchrotron beam source. Data were processed with HKL3000 (Otwinowski and Minor, i997). A total of 3 selenium and 1 zinc sites were located by the SHELXD (Schneider and Sheldrick, 2002) program using MAD data between 50-3.0 Å resolution range. The SHARP program was used to calculate the experimental and model-combined phases using the MAD data in the resolution range of 50-2.3 Å as well as for density modification. The model was built with O using the high quality electron density map obtained, and was refined with CNS to 2.3 Å resolution with excellent statistics. The final refinement statistics and geometry as defined by Procheck were in good agreement and are summarized in Table 1. Structure figures were designed using PyMOL (DeLano, 2002).

Construction of Apo3G Mutants

Mutant Apo3G proteins (D316R1D3 17R, R3 13E/R320D, and R374E/R376D) were constructed by site-directed mutagenesis using the pAcG2T-Apo3G vector as the template. The following primers and their complementary strands were used: 5′ctt cac tgc ccg cat cta tag aag aca agg aag atg tca gga g 3′ (D3 16R/D3 17R), 5′ctg tgc atc ftc act gcc gag atc tat gat gat caa gga gat tgt cag gag ggg ctg cgc 3′ (R313E/R320D), and 5′gag cac agc caa gac ctg agt ggg gag ctg gac gcc aft ctc cag aat cag g 3′ (R374E/R376D). The entire coding region of Apo3G mutant constructs was verified by DNA sequencing. The mutant plasmids were then cotransfected, according to the manufacturer's protocol, with linearized baculovirus DNA (BD Biosciences) to generate recombinant mutant Apo3G baculovirus. Wild-type and mutant Apo3G expression in Sf9 insect cells and purification was carried out as described previously (Chelico et al., 2008). Mutant E. coli GST-Apo3G proteins (R213E, R215E, K249E, R256E, W285A, F289A, Y315A) were constructed by site directed mutagenesis using the pGEX-6P1-GST-Apo3G vector as the template. The following primers and their complementary strands were used: 5′ aat gaa cct tgg gil gaa ggt cgt cac gag act tac 3′ (R213E), 5′ gaa ccttgg gil cgt ggt gaa cac gag acttac ctg 3′ (R215E), 5′ tgt aac cag gcc ccg cac gag cac ggt ttt ctg gaa 3′ (K249E), 5′ g cac ggt ttt ctg gaa ggt gaa cac gcc gaa ctg tg 3′ (R256E), 5′ gil acc tgc ttt acc tct gcg tcc ccg tgc ttt tcc 3′ (W285A), 5′ acc tct tgg tcc ccg tgc get tcc tgc gca caa gaa 3′ (F289A), 5′ atc ftc act gca cgt aft gcc gac gac cag ggc cgt 3′ (Y315A). The entire coding region of Apo3G mutant constructs was verified by DNA sequencing. Plasmids were expressed in XA-90 E. coli cells and were lysed by French press. Further purification was carried out as described previously (Chelico et al., 2008).

DNA Binding

Apo3G-DNA binding were monitored by changes in steady state fluorescence depolarization (rotational anisotropy). Reaction mixtures (70 μl), containing an F-labeled DNA (SO nM) in buffer (50 mM HEPES, pH 7.3, 1 mM DTT and 5 mM MgCl₂) and varying concentration of 0 to 500 nM Apo3G, were incubated at 37° C. The sequence of the ssDNA is: tta gat gag tgt aa(FdT) gtg ata tat gtg tat. Rotational anisotropy was measured as described previously (Chelico et al., 2006). The fraction of DNA bound to protein was determined as described previously (Bertram et al., 2004).

Deamination Activity

Apo3G (0.024-μM) was allowed to react with 500 nM FdT incorporated ssDNA for 10 or 15 mm and subsequently treated with UDG and resolved on 16% UREA PAGE for analysis as described previously¹⁰. Specific activity, measured as fmoles substrate deaminated per pg enzyme per minute, was calculated from the percent deamination of an ssDNA substrate over a range of enzyme concentrations. For experiments measuring processivity and directionality the ssDNA substrate sequence is: 5′ aaa gag aaa gtg ata ccc aaa gag taa agt (FdT) aga tag aga gtg ata ccc aaa gag taa agt tag taa gat gtg taa gta tgt taa 3′. For specific activity measurements the ssDNA substrate sequence is: gg (FdT) agt tta gtg gtt tgt ata gaa tta ata ccc aaa gaa gtg tat gta att gtt atg ata aga ttg aaa.

### REFERENCES

Bertram, J. G., Bloom, L. B., ODonnell, M., and Goodman, M. F. (2004). Increased dNTP binding affinity reveals a nonprocessive role for Escherichia coli beta clamp with DNA polymerase IV. Journal of Biological Chemistry 279, 33047-33050.

Betts, L., Xiang, S., Short, S. A., Wolfenden, R., and Carter, C. W. (1994). Cytidine deaminase. The 2.3 A crystal structure of an enzyme: transition-state analog complex. Current Biology 235, 635-656.

Bogerd, H. P., Doehle, B. P., Wiegand, H. L., and Cullen, B. R. (2004). A single amino acid difference in the host APBEC3G protein controls the primate species specificity of HIV type 1 virion infectivity factor. Proc Natl Acad Sci 101, 3770-3774.

Bransteitter, Sneeden, J. L., Allen, S., Pham, P., and Goodman, M. F. (2006). First AID (Activation-induced Cytidine Deaminase) Is Needed to Produce High Affinity Isotype-switched Antibodies. Journal of Biological Chemistry 281, 16833-16836.

Burnett, A., and Spearman, P. (2007). APOBEC3G Multimers Are Recruited to the Plasma Membrane for Packaging into Human Immunodeficiency Virus Type 1 Virus-Like Particles in an RNA-Dependent Process Requiring the NC Basic Linker. Journal of Virology 81, 5000-50 13.

Chelico, L., Sachio, E J., Erie, D A., and Goodman, M. F. (2008). A model for oligomeric regulation of APOBEC3G cytosine deaminase-dependent restriction of HIV. Journal of Biological Chemistry printed online, 10.1074/jbc.M801004200.

Chelico, L., Pham, P., Calabrese, P., and Goodman, M. F. (2006). APOBEC3G DNA deaminase acts processively 3 to S on single-stranded DNA. Nature Structural and Molecular Biology 13, 392-399.

Chen, K. M., Harjes, E., Gross, P. J., Fahmy, A., Lu, Y., Shindo, K., Harris, R. S., and Matsuo, H. (2008). Structure of the DNA deaminase domain of the HIV-1 restriction factor APOBEC3G. Nature 452, 116-119.

Chen, K. M., Martemyanova, N., Lu, Y., Shindo, K., Matsuo, H., and Harris, R. 5. (2007). Extensive mutagenesis experiments corroborate a structural model for the DNA deaminase domain of APOBEC3G. Federation of European Biochemical Societies 581, 4761-4766.

Chiu, Y. L., and Greene, W. C. (2007). The APOBEC3 Cytidine Deaminases: An Innate Defensive Network Opposing Exogenous Retroviruses and Retroelements. Annu Rev Immunol 26, 317-353.

Chung, S. J., Fromme, J. C., and Verdine, G. L. (2005). Structure of human cytidine deaminase bound to a potent inhibitor. Journal of Medicinal Chemistry 48, 658-660.

Conticello, S. G., Langlois, M. A., and Neuberger, M. S. (2007a). Insights into DNA deaminases. Nature Structural and Molecular Biology 14, 7-9.

Conticello, S. G., Langlois, M. A., Yang, Z., and Neuberger, M. S. (2007b). DNA Deamination in Immunity: AID in the Context of Its APOBEC Relatives. Advances in Immunology 94, 37-73.

Dance, G. S., Beemiller, P., Yang, Y., Mater, D. V., Mian, I. S., and Smith, H. S. (2001). Identification of the yeast cytidine deaminase CDD1 and an orphan C→U RNA editase. Nucleic Acids Research 29, 1772-1780.

DeLano, W. L. (2002). The PyMOL Molecular Graphics System, DeLano Scientific).

Guo, F., Cen, S., Niu, M., yANG, y., Gorelick, R., and Kleiman, L. (2007). The Interaction of APOBEC3G with Human Immunodeficiency Virus Type 1 Nucleocapsid Inhibits tRNA3Lys Annealing to Viral RNA. Journal of Virology 81, 11322-11331.

Holmes, R. K., Malim, M. H., and Bishop, K. N. (2007). APOBEC-mediated viral restriction: not simply editing? Trends in Biochemical Sciences 32, 118-128.

Huthoff, H., and Malim, M. H. (2007). Identification of amino acid residues in APOBEC3G required for regulation by human immunodeficiency virus type 1 Vif and Virion encapsidation. Journal of Virology 81,3807-3815.

Iwatani, Y., Chan, D. S. B., Wang, F., Maynard, K. S., Sugiura, W., Gronenborn, A. M., Rouzina, I., Williams, M. C., Musier-Forsyth, K., and Levin, J. G. (2007). Deaminase-independent inhibition of HIV-1 reverse transcription by APOBEC3G. Nucleic Acids Research 35, 7096-7108.

Jarmuz, A., Clhester, A., Bayliss, J., Gisbourne, J., Dunham, I., Scott, J., and Navaratnam, N. (2002). An anthropoid-specific locus of orphan C to U RNA-editing enzymes on chromosome 22. Genomics 79, 285-296.

Losey, H., Ruthenburg, A. J., and Verdine, G. L. (2006). Crystal structure of Staphylococcus aureus tRNA adenosine deaminase TadA in complex with RNA. Nature Structural and Molecular Biology 13, 153-159.

Luo, K., Wang, T., Liu, B., Tian, C., Xiao, Z., Kappes, J., and Yu, X. (2007). Cytidine Deaminases APOBEC3G and APOBEC3F Interact with Human Immunodeficiency Virus Type 1 Integrase and Inhibit Proviral DNA Formation. Journal of Virology 81, 7238-7248.

Mangeat, B., Turelli, P., Liao, S., and Trono, D. (2004). A Single Amino Acid Determinant Governs the Species-specific Sensitivity of APOBEC3G to Vif Action. Journal of Biological Chemistry 15, 14481-14483.

Mariani, R., Chen, D., Schrofelbauer, B., Navarro, F., Konig, R., Bollman, B., Munk, C., Nymark-McMahon, H., and Landau, N. R. (2003). Species-specific exclusion of APOBEC3G from HIV-1 virions by Vif. Cell 114, 21-31.

Mbisa, J. L., Barr, R., Thomas, J. A., Vandegraaff, N., Dorweiler, I. J., Svarovskaia, E. S., Brown, W. L., Mansky, L. M., Gorelick, R. J., Harris, R. S., et al. (2007). Human Immunodeficiency Virus Type 1 cDNAs Produced in the Presence of APOBEC3G Exhibit Efects in Plus-Strand DNA Transfer and Integration. Journal of Virology 81, 7099-7110.

Miyagi, E., Opi, S., Takeuchi, H., Khan, M., Goila-Gaur, R., Kao, S., and Strebel, K. (2007). Enzymatically Active APOBEC3G Is Required for Efficient Inhibition of Human Immunodeficiency Virus Type 1. Journal of Virology 81, 13346-13353.

Navaratnam, N., Fujino, T., Bayliss, J., Jarmuz, A., How, A., Richardson, N., Somasekaram, A., Bhattacharya, S., Carter, C., and Scott, J. (1998). Escherichia coli cytidine deaminase provides a molecular model for ApoB RNA editing and a mechanism for RNA substrate recognition. Journal of Molecular Biology 275, 695-714.

Otwinowski, Z., and Minor, W. (1997). Processing of X-ray Diffraction Data Collected in Oscillation Mode. Methods in Enzymology 276, 307-326.

Peled, J. U., Kuang, F. L., Iglesias-Ussel, M. D., Roa, S., Kalis, S. L., Goodman, M. F., and Scharff, M. D. (2007). The Biochemistry of Somatic Hypermutation. Annu Rev Immunol 26, 481-511.

Prochnow, C., Bransteitter, R., Klein, M. G., Goodman, M. F., and Chen, X. 5. (2007). The APOBEC-2 crystal structure and functional implications for the deaminase AID. Nature 445, 447-451.

Schneider, T. R., and Sheldrick, G. M. (2002). Substructure solution with SHELXD. Acta Crystallographica 58, 1772-1779.

Schumacher, A. J., Hache, G., Macduff, D. A., Brown, W. L., and Harris, R. 5. (2008). The DNA deaminase activity of human APOBEC3G is required for Tyi, MusD, and human immunodeficiency virus type 1 restriction. Journal of Virology 82, 2652-2660.

Suspene, R., Sommer, P., Henry, M., Ferris, S., Guetard, D., Pochet, S., Chester, A., Navaratnam, N., Wain-Hobson, S., and Vartanian, J. P. (2004). APOBEC3G is a single-stranded DNA cytidine deaminase and functions independently of HIV reverse transcriptase. Nucleic Acids Research 32, 242 1-2429.

Wang, W., and Malcolm, B. A. (1999). Two-stage PCR protocol allowing introduction of multiple mutations, deletions and insertions using QuikChange site-directed mutatgenesis. BioTechniques 26, 680-682.

Wedekind, J. E., Dance, G. S., Sowden, M. P., and Smith, H. C. (2003). Messenger RNA editing in mammals: new members of the APOBEC family seeking roles in the family business. Trends in Genetics 19, 207-216.

Wedekind, J. E., Gillilan, R., Janda, A., Krucinska, J., Salter, J. D., Bennett, R. P., Raina, J., and Smith, H. C. (2006). Nanostructures of APOBEC3G support a hierarchical assembly model of high molecular ribonucleoprotein particles from dimeric subunits. Journal of Biological Chemistry 281, 38122-38126.

Xiao-Yu, L., Guo, F., Zhang, L., and Kleiman, L. (2007). APOBEC3G Inhibits DNA Strand Transfer during HIV-1 Reverse Transcription. Journal of Biological Chemistry 282, 32065-32074.

Xie, K., Sowden, M. P., Dance, G. S., Torelli, A. T., Smith, H. C., and Wedekind, J. E. (2004). The structure of a yeast RNA-editing deaminase provides insight into the fold and function of activation-induced deaminase and APOBEC-1. Proc Natl Acad Sci 101, 8114-8119.

Xu, H., Svarovskaia, E. S., Barr, R., Zhang, Y., Khan, M. A., Strebel, K., and Pathak, V. K. (2004). A single amino acid substitution in human APOBEC3G antiretroviral enzyme confers resistance to HIV-1 virion infectivity factor-induced depletion. Proc Natl Acad Sci 101, 5652-5657.

Yu, Q., Konig, R., Pillai, S., Chiles, K., Kearney, M., Palmer, S., Richman, D., Coffin, J. M., and Landau, N. R. (2004). Single-strand specificity of APOBEC3G accounts for minus-strand deamination of the HIV genome. Nature Structural and Molecular Biology 11, 435-442.

Zhang, K., Mangeat, B., Ortiz, M., Zoete, V., Trono, D., Telenti, A., and Michielin, 0. (2007). Model Structure of Human APOBEC3G. Public Library of Science Biology 2, 1-7.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

What is claimed is:

1. A method for identifying a compound that binds to any fragment of an APOBEC protein, the method comprising:
(a), obtaining the three dimensional structure of the APOBEC-3G-CD2 monomer protein; and
(b) identifying or designing one or more compounds that bind, mimic, enhance, disrupt, or compete with interactions of APOBEC family proteins with themselves, their nucleic acid substrates and other cellular or viral proteins based on the three dimensional structure of the APOBEC-3G-CD2 monomer protein.

2. The method of claim 1, further comprising contacting one or more compounds identified in step (b) with an APOBEC family protein or the APOBEC-3G-CD2 monomer protein.

3. The method of claim 2, further comprising measuring the activity of an APOBEC family protein or the APOBEC-3G-CD2 monomer protein, when the APOBEC family protein or the APOBEC-3G-CD2 monomer protein is contacted with the one or more compounds.

4. The method of claim 3, further comprising comparing activities of an APOBEC family protein or the APOBEC-3G-CD2 monomer protein when the APOBEC family protein or the APOBEC-3G-CD2 monomer protein is in the presence of and in the absence of the one or more compounds.

5. The method of claim 1, further comprising contacting one or more compounds identified in step (b) with a cell that expresses an APOBEC family protein or the APOBEC-3G-CD2 protein and detecting whether a phenotype of the cell changes when the one or more compounds are present.

6. The method of claim 1, wherein a therapeutically effective amount of the one or more compounds is effective at restricting the replication of one or more viruses associated with one or more conditions selected from the group of Human Immunodeficiency Virus (HIV) and Hepatitis B virus (HBV).

7. The method of claim 1, wherein a therapeutically effective amount of the one or more compounds is effective at treating Hyper-IgM-2 Syndrome, B cell lymphomas.

8. The method of claim 1, wherein the viral proteins are HIV Vif.

9. A method for identifying a compound that binds to any fragment of an APOBEC family protein that bears similarity with a root-mean-square deviation (RMSD) of 2.0 with the APOBEC-3G-CD2 monomer structure the method comprising:
(a), obtaining the three dimensional structure of an APOBEC family protein that bears similarity with a root-mean-square deviation (RMSD) of 2.0 with the APOBEC-3G-CD2 monomer structure; and
(b) identifying or designing one or more compounds that bind, mimic, enhance, disrupt, or compete with interactions of APOBEC family proteins with themselves, their nucleic acid substrates and other cellular or viral proteins based on the three dimensional structure of an APOBEC family protein that bears similarity with a root-mean-square deviation (RMSD) of 2.0 with the APOBEC-3G-CD2 monomer structure.

10. The method according to claim 9, further comprising measuring an activity of an APOBEC family protein that bears similarity with a root-mean-square deviation (RMSD) of 2.0 with an APOBEC-3G-CD2 monomer structure when the APOBEC family protein is contacted with the one or more compounds.

11. The method according to claim 10, further comprising comparing activities of an APOBEC family protein that bears similarity with a root-mean-square deviation (RMSD) of 2.0 with an APOBEC-3G-CD2 monomer when the APOBEC family protein is in the presence of and in the absence of the one or more compounds.

12. The method according to claim 11, further comprising contacting one or more compounds identified in step (b) with a cell that expresses an APOBEC family protein that bears similarity with a root-mean-square deviation (RMSD) of 2.0 with an APOBEC-3G-CD2 monomer and detecting whether a phenotype of the cell changes when the one or more compounds are present.

13. The method of claim 9, wherein a therapeutically effective amount of the one or more compounds is effective at restricting the replication of one or more viruses associated with one or more conditions selected from the group of Human Immunodeficiency Virus (HIV) and Hepatitis B virus (HBV).

14. The method of claim 9, wherein a therapeutically effective amount of the one or more compounds is effective at treating one or more conditions selected from the group of Human Hyper-IgM-2 Syndrome and B cell lymphomas.

15. The method of claim 9, wherein the viral proteins are HIV Vif proteins.

16. A method of treating HIV or AIDS in mammals comprising:
(a) identifying one or more compounds that bind, mimic, enhance, disrupt, or compete with interactions of APOBEC family proteins with themselves, their nucleic acid substrates and other cellular or viral proteins based on the three dimensional structure of an APOBEC family protein that bears similarity with a root-mean-square deviation (RMSD) of 2.0 with the APOBEC-3G-CD2 monomer structure; and
(b) providing a therapeutically effective amount of the one or more compounds to a mammal to treat HIV or AIDS.

17. The method of claim 16, wherein the therapeutically effective amount of the one or more compounds treats HIV or AIDS by interfering with the RNA binding of the HIV virus.

18. The method of claim 16, wherein the viral proteins are HIV Vif proteins and the therapeutically effective amount of the one or more compounds treats HIV or AIDS by preventing HIV Vif protein mediation of APOBEC enzymes that restrict HIV replication.

19. The method of claim 16, wherein the viral proteins are HIV Vif proteins and the therapeutically effective amount of the one or more compounds binds to the APOBEC family proteins that inhibits interactions with the Vif protein and restore the ability of APOBEC family proteins to restrict HIV viral replication.
